SEQUENCE LISTING (19 sequences)

SEQ ID NO: 1 - DNA, 974 bp, Rattus rattus
aattcggcacgagccaaccttgagcatctgatcctcttggcttctccttcctatcgctgagctggtaggctggagacagttgtttgggtgccaacatcaacaaacgatttctgtagtttagcgtttatgaccctggcgtgaagatttaaggtctggattaagcctgttgacttctccagctacttctaaatttttgtgcataggtgctctggtctctgttgctgcttagttcttccagccttcctcaatgccagttttataatatgcaggtctctcccctcagtaatccaggatggctactctgatctttgttgataaggataacgaagagccaggcagccgtttggcatctaaggatggattgaagctgggctctggtgtcaaagccttagatgggaaattgcaggtttcaacgccacgagtcggcaaagtgttcggtgccccaggcttgcctaaagccagcaggaaggctctgggaactgtcaacagagttactgaaaagccagtgaagagtagtaaacccctgcaatcgaaacagccgactctgagtgtgaaaaagatcaccgagaagtctactaagacacaaggctctgctcctgctcctgatgatgcctacccagaaatagaaaagttcttccccttcgatcctctagattttgagagttttgacctgcctgaagagcaccagatctcacttctccccttgaatggagtgcctctcatgatcctgaatgaagagagggggcttgagaagctgctgcacctggaccccccttcccctctgcagaagcccttcctaccgtgggaatctgatccgttgccgtctcctcccagcgccctctccgctctggatgttgaattgccgcctgtttgttacgatgcagatatttaaacgtcttactcctttatagtttatgtaagttgtattaataaagcatttgtgtgtaaaaaaaaaaaaaaaaactcgagagtac

SEQ ID NO: 2 - protein, 199 aa, Rattus rattus
MATLIFVDKDNEEPGSRLASKDGLKLGSGVKALDGKLQVSTPRVGKVFGAPGLPKASRKALGTVNRVTEKPVKSSKPLQSKQPTLSVKKITEKSTKTQGSAPAPDDAYPEIEKFFPFDPLDFESFDLPEEHQISLLPLNGVPLMILNEERGLEKLLHLDPPSPLQKPFLPWESDPLPSPPSALSALDVELPPVCYDADI

SEQ ID NO: 3 - DNA, 779 bp, Homo sapiens
atggccgcgagttgtggtttaaaccaggagtgccgcgcgtccgttcaccgcggcctcagatgaatgcggctgttaagacctgcaataatccagaatggctactctgatctatgttgataaggaaaatggagaaccaggcacccgtgtggttgctaaggatgggctgaagctggggtctggaccttcaatcaaagccttagatgggagatctcaagtttcaacaccacgttttggcaaaacgttcgatgccccaccagccttacctaaagctactagaaaggctttgggaactgtcaacagagctacagaaaagtctgtaaagaccaagggacccctcaaacaaaaacagccaagcttttctgccaaaaagatgactgagaagactgttaaagcaaaaagctctgttcctgcctcagatgatgcctatccagaaatagaaaaattctttcccttcaatcctctagactttgagagttttgacctgcctgaagagcaccagattgcgcacctccccttgagtggagtgcctctcatgatccttgacgaggagagagagcttgaaaagctgtttcagctgggccccccttcacctgtgaagatgccctctccaccatgggaatccaatctgttgcagtctccttcaagcattctgtcgaccctggatgttgaattgccacctgtttgctgtgacatagatatttaaatttcttagtgcttcagagtttgtgtgtatttgtattaataaagcattctttaacagataaaaaaaaaaaaaaaaa

SEQ ID NO: 4 - protein, 202 aa, Homo sapiens
MATLIYVDKENGEPGTRVVAKDGLKLGSGPSIKALDGRSQVSTPRFGKTFDAPPALPKATRKALGTVNRATEKSVKTKGPLKQKQPSFSAKKMTEKTVKAKSSVPASDDAYPEIEKFFPFNPLDFESFDLPEEHQIAHLPLSGVPLMILDEERELEKLFQLGPPSPVKMPSPPWESNLLQSPSSILSTLDVELPPVCCDIDI

SEQ ID NO: 5 - DNA, 31 bp, Artificial Sequence
  Synthetic oligonucleotide.
gatgctctccgcactctgggaatccaatctg

SEQ ID NO: 6 - DNA, 32 bp, Artificial Sequence
  Synthetic oligonucleotide.
ttcacaagttgaggggcgcccagctgaaacag

SEQ ID NO: 7 - DNA, 32 bp, Artificial Sequence
  Synthetic oligonucleotide specific to pCI-neo plasmid. vector.
ggctagagtacttaatacgactcactataggc

SEQ ID NO: 8 - DNA, 31 bp, Homo sapiens
ctatgtcacagcaaacaggtggcaattcaac

SEQ ID NO: 9 - protein, 56 aa, Homo sapiens [sequence omitted]

SEQ ID NO: 10 - DNA, 168 bp, Homo sapiens
atgatccttgacgaggagagagagcttgaaaagctgtttcagctgggccccccttcacctgtgaagatgccctctccaccatgggaatccaatctgttgcagtctccttcaagcattctgtcgaccctggatgttgaattgccacctgtttgctgtgacatagatatt

SEQ ID NO: 11 - DNA, 16 bp, Artificial Sequence
  Anchored primer sequence.
aagctttttttttttg

SEQ ID NO: 12 - DNA, 13 bp, Artificial Sequence
  Arbitrary primer sequence.
aagcttgctgctc

SEQ ID NO: 13 - DNA, 16 bp, Artificial Sequence
  n = a, g, or c; Anchored primer sequence.
aagctttttttttttn

SEQ ID NO: 14 - protein, 194 aa, Mus musculus
MATLIFVDKDNEEPGRRLASKDGLKLGTGVKALDGKLQVSTPRVGKVFNAPAVPKASRKALGTVNRVAEKPMKTGKPLQPKQPTLTGKKITEKSTKTQSSVPAPDDAYPEIEKFFPFNPLDFDLPEEHQISLLPLNGVPLITLNEERGLEKLLHLGPPSPLKTPFLSWESDPKPPSALSTLDVELPPVCYDADI

SEQ ID NO: 15 - DNA, 945 bp, Mus musculus
tcttgaacttgttatgtagcaggaggccaaatttgagcatcctcttggcttctctttatagcagagattgtaggctggagacagttttgatgggtgccaacataaactgatttctgtaagagttgagtgttttatgaccctggcgtgcagatttaggatctggattaagcctgttgacttctccagctacttataaatttttgtgcataggtgccctgggtaaagcttggtctctgttactgcgtagtttttccagccgtctcaatgccaatattcaggctctctcccttagagtaatccagaatggctactcttatctttgttgataaggataatgaagaacccggccgccgtttggcatctaaggatgggttgaagctgggcactggtgtcaaggccttagatgggaaattgcaggtttcaacgcctcgagtcggcaaagtgttcaatgctccagccgtgcctaaagccagcagaaaggctttggggacagtcaacagagttgccgaaaagcctatgaagactggcaaacccctccaaccaaaacagccgaccttgactgggaaaaagatcaccgagaagtctactaagacacaaagctctgttcctgctcctgatgatgcctacccagaaatagaaaagttcttccctttcaatcctctagattttgacctgcctgaggagcaccagatctcacttctccccttgaatggcgtgcctctcatcaccctgaatgaagagagagggctggagaagctgctgcatctgggcccccctagccctctgaagacaccctttctatcatgggaatctgatccgctgtactctcctcccagtgccctctccactctggatgttgaattgccgcctgtttgttacgatgcagatatttaaacttcttacttctttgtagtttctgtatgtatgttgtattaataaagcatt

SEQ ID NO: 16 - protein, 56 aa, Rattus rattus
MILNEERGLEKLLHLDPPSPLQKPFLPWESDPLPSPPSALSALDVELPPVCYDADI

SEQ ID NO: 17 - protein, 56 aa, Mus musculus
ITLNEERGLEKLLHLGPPSPLKTPFLSWESDPLYSPPSALSTLDVELPPVCYDADI

SEQ ID NO: 18 - DNA, 168 bp, Rattus rattus
atgatcctgaatgaagagagggggcttgagaagctgctgcacctggaccccccttcccctctgcagaagcccttcctaccgtgggaatctgatccgttgccgtctcctcccagcgccctctccgctctggatgttgaattgccgcctgtttgttacgatgcagatatt

SEQ ID NO: 19 - DNA, 168 bp, Mus musculus
atcaccctgaatgaagagagagggctggagaagctgctgcatctgggcccccctagccctctgaagacaccctttctatcatgggaatctgatccgctgtactctcctcccagtgccctctccactctggatgttgaattgccgcctgtttgttacgatgcagatatt

(19) United States
(12) Patent Application Publication
(10) Pub. No.: US 2002/0147162 A1
(43) Pub. Date: Oct. 10, 2002

(54) Methods of modulating angiogenesis by regulating the expression of pituitary tumor transforming gene (PTTG)

(76) Inventors: Anthony P. Heaney, Los Angeles, CA (US); Hiroki Ishikawa, Nagasaki (JP); Run Yu, Los Angeles, CA (US); Gregory A. Horwitz, Los Angeles, CA (US); Xun Zhang, Malden, MA (US); Shlomo Melmed, Los Angeles, CA (US)

Correspondence Address: Edward G. Poplawski, Esq., SIDLEY AUSTIN BROWN & WOOD, 555 West Fifth Street, Los Angeles, CA, 90013-1010, US

(21) Appl. No.: 09/777,422

(22) Filed: Feb. 5, 2001

Related U.S. Application Data
(63) Continuation-in-part of application No. 09/730,469, filed on Dec. 4, 2000.
Application No. 09/730,469 is a continuation-in-part of application No. 09/687,911, filed on Oct. 13, 2000.
Application No. 09/687,911 is a continuation-in-part of application No. 09/569,956, filed on May 12, 2000.
Application No. 09/569,956 is a continuation-in-part of application No. 08/894,251, filed on Jul. 23, 1999.
Application No. 08/894,251 is a 371 of international application No. PCT/US97/21463, filed on Nov. 21, 1997.
(60) Provisional application No. 60/031,338, filed on Nov. 21, 1996.

(51) Int. Cl. A61K 31/70; A01N 43/04

(52) U.S. Cl. 514/44

(57) ABSTRACT

Disclosed is a method of modulating angiogenesis in a tissue comprising mammalian cells, including cells of human origin, in vitro or in vivo. Also disclosed are a method of enhancing wound healing and/or tissue regeneration and a method of limiting scar formation.

This application is a continuation-in-part of U.S. Ser. No. 09/730,469, filed Dec. 4, 2000, which is a continuation-in-part of U.S. Ser. No.09/687,911, filed on Oct. 13, 2000, which is a continuation-in-part of U.S. Ser. No.09/569,956, filed on May 12, 2000, which is a continuation-in-part of U.S. Ser. No. 08/894,251, filed on Jul. 23, 1999, as a national stage application, under 35 U.S.C. §371, of international application PCT/US97/21463, filed Nov. 21, 1997, which claims the priority of the filing date of U.S. Provisional Application Serial No. 60/031,338, filed Nov. 21, 1996.

The U.S. Government has a paid-up license in this invention and the right in limited circumstances to require the patent owner to license others on reasonable terms as provided for by the terms of Contract CA75979, awarded by the National Cancer Institute of the National Institutes of Health.

### BACKGROUND OF THE INVENTION

Throughout the application various publications are referenced in parentheses. The disclosures of these publications in their entireties are hereby incorporated by reference in the application in order to more fully describe the state of the art to which this invention pertains.

1. Field of the Invention

The present invention relates to a method of inhibiting neoplastic cellular proliferation and/or transformation of breast and/or ovarian cells, in vitro and in vivo.

2. Related Art

Pituitary Tumor Transforming Gene (PTTG) is highly expressed in pituitary tumors and neoplasms from the hematopoietic system and colon. (Zhang, X. et al., Structure, expression, and function of human pituitary tumor-transforming gene (PTTG), Mol Endocrinol. 13:156-66 [1999a]; Zhang, X. et al., Pituitary tumor transforming gene (PTTG) expression in pituitary adenomas, J. Clin. Endocrinol. Metab. 84:761-67 [1999b]; Heaney, A. P et al., Pituitary tumor transforming gene in colorectal tumors, Lancet 355:712-15 [2000]; Dominguez, A. et al., hpttg, a human homologue of rat pttg, is overexpressed in hematopoietic neoplasms. Evidence for a transcriptional activation function of hPTTG, Oncogene 17:2187-93 [1998]; Saez, C. et al, hpttg is over-expressed in pituitary adenomas and other primary epithelial neoplasias, Oncogene 18:5473-6 [1999]). PTTG1 is expressed at low levels in most normal human tissues. (Chen, L. et al., Identification of the human pituitary tumor transforming gene (hPTTG) family: molecular structure, expression, and chromosomal localization, Gene. 248:41-50 [2000]; Heaney, A. P. et al. [2000]).

Levels of PTTG expression positively correlate with pituitary and colorectal tumor invasiveness (Zhang, X. et al. [1999b]; Heaney, A. P. et al. [2000]) and are induced by estrogen. (Heaney, A. P. et al., Early involvement of estrogen-induced pituitary tumor transforming gene and fibroblast growth factor expression in prolactinoma pathogenesis, Nat. Med. 5: 1317-21 [1999]). In tumor cells, PTTG mRNA and protein expressions are cell cycle-dependent and peak at G2/M phase. (Yu, R. et al., Pituitary Tumor Transforming Gene (PTTG) Regulates Placental JEG-3 Cell Division and Survival: Evidence from Live Cell Imaging, Mol. Endocrinol. 14:1137-1146 [2000]) The mechanism of PTTG action is not very clear. PTTG upregulates basic fibroblast growth factor secretion (Zhang, X. et al. [1999a]), and transactivates DNA transcription (Dominguez, A. et al. [1998]; Wang, Z. et al., Pituitary tumor transforming gene (PTTG) transactivating and transforming activity, J. Biol. Chem. 275.7459-61[2000]).

PTTG encodes a securin protein the expression of which causes cell transformation, induces the production of basic fibroblast growth factor (bFGF), is regulated in vitro and in vivo by estrogen, and inhibits chromatid separation. (Pei, L., and Melmed S., Isolation and characterization of a pituitary tumor transforming gene, Mol. Endocrinol. 11:433-441 [1997]; Zhang, X., et al., Structure, expression, and function of human pituitary tumor-transforming gene (PTTG), Mol. Endocrinol. 13:156-166 [1999a]; Heaney, A. P., et al., Early involvement of estrogen-induced pituitary tumor transforming gene and fibroblast growth factor expression in prolactinoma pathogenesis, Nature Med. 5:1317-1321 [1999]; Zou, H., et al., Identification of a vertebrate sister-chromatid separation inhibitor involved in transformation and turnorigenesis, Science 285:418-422 [1999]).

By dysregulating chromatid separation, PTTG overexpression also leads to aneuploidy, i.e., cells having one or a few chromosomes above or below the normal chromosome number (Zou et al. [1999]; Yu, R. et al. [2000]). At the end of metaphase, securin is degraded by an anaphase-promoting complex, releasing tonic inhibition of separin, which in turn mediates degradation of cohesins, the proteins that hold sister chromatids together. Overexpression of a nondegradable PTTG disrupts sister chromatid separation (Zou et al. [1999]) and overexpression of PTTG causes apoptosis and inhibits mitoses (Yu, R. et al. [2000]). The securin function of PTTG suggests that PTTG may also be expressed in normal proliferating cells. In adult animals and humans, PTTG mRNA is most abundant in testis (Zhang, X. et al. [1999a]); Wang, Z. et al. [2000]), an organ containing rapidly proliferating gametes.

A PTTG gene family contains at least three genes that share a high degree of sequence homology, including human PTTG1, located on chromosome 5q33. (Prezant, T. R., et al., An intronless homolog of human proto-oncogene hPTTG is expressed in pituitary tumors: evidence for hPTTG family, J. Clin Endocrinol. Metab. 84:1149-52 [1999]). Murine PTTG shares 66% nucleotide base sequence homology with human PTTG1 and also exhibits transforming ability. (Wang, Z. and Melmed, S., Characterization of the murine pituitary tumor transforming gone (PTTG) and its promoter, Endocrinology [In Press; 2000]). A proline-rich region was identified near the protein C-terminus that is critical for PTTG1's transforming activity. (Zhang, X., et al. [1999a]), as demonstrated by the inhibitory effect on in vitro transformation, in vivo tumorigenesis, and transactivation, when point mutations were introduced into the proline-rich region. Proline-rich domains may function as SH3 binding sites to mediate signal transduction of protein-tyrosine kinase. (Pawson, T., Protein modules and signaling networks, Nature 373:573-580 [1995]; Kuriyan, J., and Cowburn, D., Modular peptide recognition domains in eukaryotic signaling, Annu, Rev. Biophys. Biomol. Struct. 26 259-288 [1997]).

Breast and ovarian cancers are a model of hormone dependent malignancy. Estrogens and progesterone, acting via specific nuclear receptors, are necessary for normal development of mammary gland and ovarian tissue and their differentiated function. In addition to classical estrogenic ligand-estrogen receptor (ER) interactions, and subsequent ER binding to estrogen-response elements to regulate gene transcription, it is now apparent that transcriptional modulation can be mediated through the membranal ER. (Levin E. R., Cellular functions of the plasma membrane estrogen receptor, TEM 10:374-77 [1999]) This action requires modification of cytosolic signal transduction pathways such as extracellular-signal-regulated kinase/mitogen-activated protein kinase pathways (ERK/MAPK).

In breast and ovarian cancers, the molecular mechanisms through which these signal transduction effects are mediated are not well defined, although c-myc and cyclin D1 have been identified as major downstream targets of estrogen and progestin-stimulated cell cycle progression. In addition to regulating cyclin abundance, recruitment of specific CDK inhibitors, such as p21 is impaired by estrogen, and additional, as yet undefined estrogen-regulated components are likely to be regulators of mammary epithelial cell proliferation and differentiation. (Sutherland, R. L., et al., Estrogen and progestin regulate cell cycle progression, J. Mammary Gland Biol. Neoplasia 3:63-72 [1998]).

Several studies have described the involvement of SP 1 and half-site EREs in conferring estrogen-responsiveness of several genes, including creatine kinase B, c-myc, the retinoic acid receptor α, heat shock protein 27. (Wu-Peng X. et al., Delineation of sites mediating estrogen regulation of the rat creatine kinase B gene, Mol. Endocrinol 6:231-240 [1992]; Dubik, D. and Shiu, R, Mechanism of estrogen activation of c-myc oncogene expression, Oncogene 7:1587-1594 [1992]). This cooperative interaction of a half-site ERE and an SP1 site has recently been described for the progesterone receptor (Petx, L. and Nardulli, A. M., Sp1 binding sites and an estrogen response element half-site are involved in regulation of the human progesterone receptor A promoter, Mol. Endocrinol. 14:972-85 [2000]). In the context of complex promoters, EREs are generally found in multiple copies or encased among binding motifs for other transcription factors (Porter, W. et al., Functional synergy between the transcription factor Sp1 and the estrogen receptor, Mol. Endo. 11:1569-80 [1997]). It has been demonstrated that the SP1 sites on the murine and human PTTG-promoter are crucial for its transactivation activity, and mutational disruption of the SP1 element or competition with a known SP1 oligo resulted in up to 90% loss of PTTG-promoter activity. (Wang, Z. and Melmed, S., SP1 activates the pituitary tumor transforming gene (PTTG) promoter during cellular transformation J Biol Chem [2000]; Kakar, S. S., Molecular cloning, genomic organization, and identification of the promoter for the human pituitary tumor transforming gene (PTTG), Gene 240: 317-324 [1999]).

In many solid tumors, tumor vascularity may inversely correlate with prognosis, and both bFGF and VEGF expression have been reported to predict prognosis (Takahashi, Y. et al., Expression of vascular endothelial growth factor and its receptor, KDR, correlates with vascularity, metastasis, and proliferation of human colon cancer, Cancer Res 55:3964-68 [1995]). Quantification of angiogenesis in breast cancer can be used as an independent prognostic factor. (Weidner, N. et al., Tumor angiogenesis: a new significant and independent prognostic factor in early-stage breast carcinoma, J Natl. Cancer Inst 84:1875-1887 [1992]; Horak, E. R. et al., Angiogenesis, assessed by platelet/endothelial cell adhesion molecule antibodies, as indicator of node metasteses and survival in breast cancer, Lancet 340:1120-1124 [1992]). Not only are tumor growth, progression, and metastasis dependent on access to vasculature, but it is also apparent that during the transition from mid-late dysplasia, as in the case of cervical intraepithelial neoplasia II (CIN II) to CIN III, an “angiogenic switch” is activated and changes in tissue angiogenic phenotype probably precede the histological tissue transition. (Hanahan, D. and Folkman, J., Patterns and emerging mechanisms of the angiogenic switch during tumorigenesis. Cell. 86:353-64 [1996])

The sequence of events in angiogenesis leading to formation of new blood vessels from pre-existing vessels is highly regulated (Jain, R K et al., Quantitative angiogenesis assays: progress and problems, Nat Med. 3:1203-1208 [1997]; Darland DC and D'Amore Pa., 1999 Blood vessel maturation: vascular development comes of age. J Clin Invest 103:157-158 [1999]), and involves dissolution of vessel basement membranes, and formation of new lumen and pericytes by vascular endothelial cells. During tumor-associated angiogenesis, sustained production of angiogenic factors by cancer cells, or indirect macrophage stimulation, causes disregulated immature vessel growth (Folkman, J. and Shing, Y., Angiogenesis, J Biol Chem. 267:10931-10934 [1992]). A number of in vitro and in vivo assays have been useful for studying angiogenesis (e.g., Jain, R K et al. [1997]; Auerbach, R. et al., Assays for angiogenesis: a review, Pharmacol Ther. 51:1-11 [1991]).

Several cytokines and growth factors, including basic fibroblast growth factor (bFGF) and vascular endothelial growth factor (VEGF) modulate angiogenesis in vivo with a paracrine mode of action. (Bikfalvi, A. et al., Biological roles of fibroblast growth factor-2, Endocr. Rev. 18:26-45 [1997]; Ferrara, N. and Davis-Smyth, T., The biology of vascular endothelial growth factor, Endocr Rev 18:4-25 [1997]). bFGF and VEGF levels in cytosolic fractions are significantly associated with intratumoral vascularization. (Expression of the angiogenic factors vascular endothelial cell growth factor, acidic and basic fibroblast growth factor, tumor growth factor-1, platlet-derived endothelial cell growth factor, placenta growth factor, and pleiotrophin in human primary breast cancer and its relation to angiogenesis, Cancer Res. 57:963-69 [1997]; Linderholm, B. et al., Vascular endothelial growth factor is of high prognostic value in node-negative breast carcinoma, J. Clin. Oncol. 16:3121-28 [1998]). bFGF and VEGF have synergistic effects on angiogenesis, and bFGF modulates endothelial expression of VEGF through both autocrine and paracrine actions (Seghezzi, G. et al., Fibroblast growth factor-2 (FGF-2) induces vascular endothelial growth factor (VEGF) expression in the endothelial cells of forming capillaries: An autocrine mechanism contributing to angiogenesis, J. Cell. Biol 141(7):1659-73 [1998]).

PTTG regulates bFGF mRNA and protein extracellular release or secretion, and this function requires a preserved C-terminus P-X-X-P motif (Zhang, X. et al. [1999a]). It has also been reported that rat pituitary pttg is regulated in vivo and in vitro by estrogen, and the maximal induction of rat pituitary pttg mRNA in vivo occurred early in pituitary transformation (normal cell to hypertrophic/hyperplastic cell), coincident with bFGF and, vascular endothelial growth factor (VEGF) induction, and pituitary angiogenesis. (Heaney, A. P. et al. [1999]).

There remains a need for a method of modulating angiogenesis, both by way of inhibition, for example in inhibiting vascularization of solid malignant tumors or in inhibiting the development of retinopathy, and by way of enhancing angiogenesis, for example in enhancing wound healing and the recovery from cardiovascular or cerebrovascular ischemia. This and other benefits are provided by the present invention as described herein.

### SUMMARY OF THE INVENTION

The present invention relates to compositions and methods useful for modulating angiogenesis. Included are compositions comprising PTTG peptides, PTTG carboxy-terminal peptide, or comprising a chimeric or fusion protein that contains a first PTTG carboxy-terminal peptide segment and a second cellular uptake-enhancing peptide segment. The invention also relates to compositions comprising a PTTG peptide-encoding polynucleotide, a PTTG carboxy-terminal-related polynucleotide, for example, a polynucleotide encoding a PTTG-C peptide or antisense PTTG-specific or PTTG-C-related oligonucleotides. Also included in the invention are compositions comprising expression vectors containing the PTTG gene or PTTG-C-related polynucleotides, including nucleic acids encoding PTTG peptide or PTTG-C peptides. The inventive PTTG peptides or PTTG-C peptides and inventive PTTG-encoding or PTTG-C-related polynucleotides are useful in the manufacture of pharmaceutical compositions, medicaments or medicants for inhibiting neoplastic cellular proliferation and/or transformation, which contain the inventive PTTG-specific antisense oligonucleotides, PTTG peptides, PTTG-C peptides, or PTTG-encoding or PTTG-C-related polynucleotides.

In accordance with the present invention, there are also provided PTTG peptides, PTTG carboxy-terminal (PTTG-C) peptides and PTTG-encoding or PTTG-C-related polynucleotides, which can also be isolated from other cellular components. The inventive peptides are useful in bioassays, as immunogens for producing anti-PTTG antibodies, or in therapeutic compositions containing such peptides and/or antibodies. Also provided are transgenic non-human mammals that comprise mammalian cells that comprise embodiments of the inventive PTTG-encoding or PTTG-C-related polynucleotides and express the inventive PTTG peptides or PTTG-C peptides, respectively.

In particular, the compositions and methods are useful in an inventive method of modulating angiogenesis in a tissue comprising mammalian cells, whether in vitro or in vivo. The method involves modulating PTTG gene expression and/or endogenous PTTG protein function in at least one of the cells, such that by (A) inhibiting PTTG gene expression and/or endogenous PTTG protein function in at least one of the cells, bFGF production and/or secretion in the tissue is inhibited and angiogenesis in the tissue is thereby inhibited; or (B) by enhancing PTTG gene expression and/or endogenous PTTG protein function in at least one of the cells, bFGF production and/or secretion in the tissue is enhanced and angiogenesis in the tissue is thereby enhanced.

Because, PTTG protein further mediates the expression of bFGF, i.e., production and/or secretion of bFGF, an important angiogenesis activator, the inventive method of also encompasses inhibiting angiogenesis in malignant tumor tissue. Angiogenesis activators, including bFGF and VEGF, are expressed and secreted by most human carcinoma cells. (Plate, K. H. et al., Nature 359:845-48 [1992]; Schultz-Hector, S. and Haghayegh, S., Cancer Res. 53:1444-49 [1993]; Yamanaka, Y. et al., Cancer Res. 53:5289-96 [1993], Buensing, S. et al., Anticancer Res. 15:2331-34 [1995]). As described herein, the inventive PTTG-C peptides dramatically reduce bFGF production by mammalian cells, shows that in accordance with the inventive method, the inventive PTTG-C peptides can impair new blood vessel growth, which is essential for tumor growth and retinopathy. On the other hand, and also described herein, overexpression of PTTG enhances angiogenic activity, in vitro and in vivo. Thus, the method of modulating angiogenesis is useful for treating conditions in which decreased angiogenesis is beneficial, such as in malignant tumors or retinopathy, and for treating conditions in which enhanced angiogensis is beneficial, such as for enhanced wound healing and/or tissue regeneration in surgery patients or patients with impaired wound healing ability (e.g., diabetics) or for treating cardiovascular or cerebrovascular ischemia (e.g., in heart attack or stroke victims). Thus, the invention also relates to a method of enhancing wound healing and/or tissue regeneration.

In some embodiments, the inventive method, by way of inhibiting angiogenesis, is directed to gene-based treatments that deliver PTTG carboxy-terminal-related polynucleotides to mammalian cells to inhibit the endogenous expression and function of PTTG, thereby inhibiting angiogenesis in the tissue; or, by way of enhancing angiogenesis, a PTTG peptide-encoding polynucleotide is delivered to the cell(s) in the tissue

Useful gene-based embodiments of the method of modulating angiogenesis involve delivering to the cell a composition comprising a PTTG-C-related polynucleotide that includes a base sequence that defines a PTTG carboxy-terminal peptide-encoding sequence or a PTTG peptide encoding polynucleotide sequence, respectively, or defines a degenerate sequence, or defines a sequence complementary to either of these. In accordance with the method, the polynucleotide, preferably complexed with a cellular uptake-enhancing agent, is delivered in an amount and under conditions sufficient to enter the cell, thereby modulating the production and/or secretion of bFGF, and thus modulating angiogenesis in the tissue.

Alternatively, useful peptide-based embodiments of the method of modulating angiogenesis involve delivering to a mammalian cell a composition comprising a PTTG peptide or PTTG carboxy-terminal peptide (PTTG-C), or a biologically functional fragment of either of these, preferably complexed with a cellular uptake-enhancing agent, in an amount and under conditions sufficient to enter the cell, thereby enhancing angiogenesis (via delivery of functional PTTG peptide) or inhibiting angiogenesis (via delivery of functional PTTG-C peptide).

The method, by way of inhibiting angiogenesis, can also be practiced by delivering to the mammalian cell(s) of the tissue of interest a PTTG-specific antisense oligonucleotide, which antisense oligonucleotide can be selected to target any PTTG-specific genomic or mRNA sequence (including splice variants) so as to prevent expression of functional PTTG protein. Alternatively, the method can be practiced by interfering with SH3-mediated signal transduction by blocking specific binding to SH3-binding sites on endogenous PTTG protein molecules in the cell(s). Alternatively, the method can be practiced by inhibiting PTTG expression by targeting with antisense oligonucleotides directed to any of several regulatory regions that have been identified in the PTTG promoter such as SSCA, 8182, cyclic-AMP responsive element, an estrogen responsive element, insulin response element, SP1, and GC Box, as well as others.

Also provided are antibodies that are specifically immunoreactive with PTTG proteins, or more particularly, with PTTG-C peptides. The inventive antibodies specifically bind to PTTG-C peptides. These anti-PTTG-C-specific antibodies are useful in assays to determine levels of PTTG proteins or PTTG-C peptides present in a given sample, e.g., tissue samples, biological fluids, Western blots, and the like. The antibodies can also be used to purify PTTG proteins or PTTG-C peptides from crude cell extracts and the like. Moreover, these antibodies are considered therapeutically useful to counteract or supplement the biological effect of PTTG proteins in vivo. Thus, the antibodies are useful in inhibiting angiogenesis.

Useful kits are also provided for facilitating the practice of the inventive methods.

The present invention is further described by related applications U.S. Ser. No. 09/730,469, filed Dec. 4, 2000; U.S. Ser. No. 09/687,911, filed on Oct. 13, 2000; U.S. Ser. No. 09/569,956, filed on May 12, 2000; U.S. Ser. No. 08/894,251, filed Jul. 23, 1999; international application PCT/US97/21463, filed Nov. 21, 1997; and U.S. provisional application 60/031,338, filed Nov. 21, 1996, the disclosures all of which are incorporated by reference.

### BRIEF DESCRIPTION OF THE DRAWINGS

FIG. 1 illustrates transcriptional activation in transfected NIH-3T3 cells, as mediated by pGAL4, pGAL4-VP16, pGAL4-wtPTTG, or pGAL4-mutPTTG 48 hours after transfection. Cell lysate proteins were assayed for luciferase and β-gal expression. pGAL4 was used as a negative control and pGAL4-VP16 as a positive control.

FIG. 2 shows PTTG-C and PTTG-Cpm expression in transfected tumor cells. FIG. 2A illustrates expression construct which express a C-terminal peptide of human PTTG protein (PTTGC), corresponding to amino acid residues 147-202 of SEQ. ID. NO.:4 (i.e., SEQ. ID. NO.:9), under the control of the CMV promoter (black bar). PXXP represents the proline-rich region(s) of the PTTG-C. A mutant expression vector (PTTG-Cpm), contained point mutations P163A, S165Q, P166L, P170L, P172A, and P173L. FIG. 2B includes representative 1% agarose gels of RT-PCR products of HeLa (top panel), T-47D (middle panel), and MCF-7 cells (bottom panel),showing PTTG-C and PTTG-Cpm expression. Products from reverse transcription carried out in the presence (+) or absence (−) of RT were used as template in PCR reactions. FIG. 2C shows a representative sequencing gel from RT-PCR followed by direct sequencing analysis showing PTTG-C and PTTG-Cpm expression in respective transfectants Arrows point to nucleotide changes.

FIG. 3 shows colony formation of HeLa (top row), T-47D (middle row), and MCF-7 (bottom row) cells transfected with PTTG-C or PTTG-Cpm expression vectors on soft agar. “Vector” (left column)shows cells transfected with vector pCI-neo alone; “PTTG-C” (middle column) shows cells transfected with vector pCI-neo containing PTTG C-terminal encoding cDNA; “PTTG-Cpm” (right column) shows cells transfected with vector pCI-neo containing mutant PTTG C-terminal cDNA (P163A, S165Q, P166L, P170L, P172A, and P173L).

FIG. 4 shows suppression of bFGF secretion by HeLa cells expressing PTTG-C peptide. The concentration of bFGF in conditioned medium derived from transfectants cultured for 72 h as measure by ELISA. “Vector” (two left-most bars) indicates medium conditioned by cells transfected with vector pCI-neo alone; “PTTG-C” (three middle bars) indicates medium conditioned by cells transfected with vector pCI-neo containing wtPTTG-C-terminal encoding cDNA; “PTTG-Cpm” (three right-most bars) indicates medium conditioned by transfected with vector pCI-neo containing mutant PTTG C-terminal encoding cDNA (P163A, S165Q, P166L, P170L, P172A, and P173L).

FIG. 5 shows PTTG mRNA expression in normal adult human T-cells treated with mitogen anti-CD3 antibody. T-cells were isolated and stimulated with anti-CD3 antibody for 72 hours. PTTG mRNA PTTG was measured with northern blotting and percentage of cells in S or G2/M phase was determined by FACS.

FIG. 6 shows PTTG mRNA expression in normal adult human T-cells treated with mitogen phytohemagglutinin (PHA). T-cells were isolated and stimulated with increasing concentrations of PHA for 72 hours. PTTG mRNA was measured with northern blotting and percentage of cells in S or G2/M phase was determined by FACS.

FIG. 7 shows IL-2 mRNA expression in normal adult human T cells treated with mitogen CD3 antibody. T-cells were isolated and stimulated with anti-CD3 antibody for 72 hours. IL-2 mRNA PTTG was measured with northern blotting and percentage of cells in S or G2/M phase was determined by FACS.

FIG. 8 shows cyclophilin mRNA expression in normal adult human T cells treated with mitogen CD3 antibody. T cells were isolated and stimulated with anti-CD3 antibody for 48 hours. Cyclophiline mRNA PTTG was measured with northern blotting.

FIG. 9 demonstrates PTTG mRNA expression and hydrocortisone. PHA (5 μg/mL)-stimulated normal adult human T cells were treated with hydrocortisone for 72 hours. PTTG mRNA was measured with northern blotting and percentage of cells in S or G2/M phase was determined by FACS.

FIG. 10 shows PTTG mRNA expression and cyclosporin. PHA (5 ug/ml)-stimulated normal adult human T cells were treated with cyclosporin for 72 h. PTTG mRNA was measured with northern blotting and percentage of cells in S or G2/M phase was determined by FACS.

FIG. 11 illustrates PTTG mRNA expression in leukemia cells. PTTG mRNA values and cell cycle of cycling human leukemia HL-60 (1), Jurkat T cells (2), resting (3), PHA (5 ug/mL)-stimulated, (4) anti-CD3-stimulated, and (5) normal adult human T cells were determined.

FIG. 12 shows PTTG mRNA expression and cell cycle in T cells. T cells were treated with the following conditions and PTTG mRNA and percentage of S phase were compared. (1) resting cells; (2) PHA (5 μg/mL)-stimulated; (3) anti-CD3-stimulated; (4) anti-CD3+hydrocortisone (100 nM); (5) anti-CD3+cyclosporine A (1 μg/mL); (6) anti-CD3+aphidicolin (1 μg/mL); (7) anti-CD3+nocodazole (500 ng/mL); (8) anti-CD3+TGF-β1 (10 ng/mL).

FIG. 13 shows PTTG mRNA expression in human Jurkat T cell leukemia line. Jurkat T cells were treated as described below. (1) cells kept for 48 h in 1% FBS-supplemented culture medium; (2) cells after medium change for fresh 1% FBS-supplemented; (3) cells after medium change for 10% FBS-supplemented; (4) phytohemagglutinin (PHA; 1 μg/mL)+phorbol-12-meristate-13-acetate (PMA; 50 ng/nL) in 1% FBS; (5) (PHA+PMA)+cyclosporine A (1 μg/mL); (6) (PHA+PMA)+TGF-β1 (10 ng/mL).

FIG. 14 shows representative Northern (upper panel) and western (lower panel) blot analysis comparing: (FIG. 14a) normal (N) and breast tumor (T; n=12) tissues; and (FIG. 14b) normal (N) and ovarian tumor (T; n=13) tissues, depicting expression of PTTG1 mRNA and PTTG1 protein. Hybridization with [α-³²P] dCTP probes for either PTTG1 or β actin or blotting with anti-PTTG1 antibody (1:5000) revealed PTTG overexpression in breast and ovarian carcinomas in comparison to adjacent normal tissue. JEG-3 choriocarcinoma cells served as a positive control. Left margins, molecular size; right margins, positions of mRNA products.

FIG. 15 demonstrates regulation of PTTG1 in vitro by estrogen. (FIG. 15a) MCF-7 cells and (FIG. 15b) SKOV-3 cells transiently transfected with full-length PTTG-promoter were incubated in medium containing serum pre-treated with charcoal stripped serum (CSS) (clear bars), prior to addition of medium containing 10% fetal bovine serum (WS) (filled bars), or medium containing CSS and added estrogen (hatched bars) (Diethylstilbestrol 10^{31 7} to 10⁻⁸ M) (E), with/without the anti-estrogen (hatched bars) (ICI-182780 10⁻⁷ to 10⁻⁸ M) (AE). PTTG1 mRNA was normalized for β-actin. Each bar represents mean±SEM of six dishes from three separate experiments. Statistical analysis was by ANOVA.

FIG. 16 demonstrates that wt-hPTTG C-terminus peptide inhibits colony formation in agar and sensitzies breast cancer cells to Taxol. MCF-7 cells (about 5,000) transfected with vector alone (a,b,c,d) or vector containing wt-hPTTG C-terminus-encoding DNA (e,f,g,h) were plated in agar containing vehicle only (a, e) or Taxol 10⁻¹¹ M (b, f), 10⁻¹⁰ M (c,g) or 10⁻⁹ M (d,h) (magnification×200).

FIG. 17 shows the number of colonies formed by vector-transfected and vector plus wt-hPTTG C-terminus DNA-transfected MCF-7 cells in agar following treatment (10 days) with vehicle only or Taxol (10⁻¹¹ M to 10⁻¹⁰ M).

FIG. 18 shows bFGF concentrations in conditioned media derived from transfected and non-transfected NIH3T3 cells. Wild type PTTG, mutant hPTTG or vector alone-transfected or non-transfected NIH-3T3 cells were incubated in serum-free DMEM for 48 hours and aliquots of the conditioned media collected. bFGF concentration was measured by ELISA. WT, WT-hPTTG-CM; M, Mut-hPTTG-CM; C, C-CM; N, N-CM. The data shown is the mean±SD of three separate experiments. *, p<0.01 versus WT-hPTTG-CM.

FIG. 19 demonstrates endothelial cell proliferation. HUVECs were cultured on 48-well culture plates in 500 ml conditioned media derived from transfected or non-transfected NIH3 T3 cells. Cell numbers were determined after 48 hours incubation. The depicted results are the mean±SD of three separate experiments. From left to right: D, serum-free DMEM; F, 1 ng/mL bFGF in DMEM; WT, WT-hPTTG-CM; M, M-hPTTG-CM; C, C-CM; N, N-CM. p<0.01 versus * serum-free DMEM, ** WT-hPTTG-CM, *** respective conditioned media alone.

FIG. 20 demonstrates migration of HUVECs in wound assay. The wounded monolayer of HUVEC was exposed for 16 hours to respective aliquots of conditioned media Migrated cells were then fixed, stained and photographed. (A) Representative micrographs of migrated HUVECs in WT-hPTTG-CM are shown. a, conditioned media alone; b, conditioned media+100 ng/ml anti-bFGF antibody. (B) Quantification of migrated HUVECs. The number of cells within 0.1×2.5-mm area in three fields was counted using the original mark made by razor blade as origin. The results shown are the average number of cells±SD per field of three separate experiments. a, control; b, WT-hPTTG-CM; c, M-hPTTG-CM; d, C-CM; e, N-CM; open circle, 1 ng/ml bFGF in DMEM; open triangle, serum-free DMEM; closed circle, conditioned medium alone; closed triangle, conditioned medium+100 ng/ml anti-bFGF antibody; closed square, conditioned medium+100 ng/ml pre-immune goat IgG.

FIG. 21A shows migration of HUVECs in a modified Boyden chamber assay. The sample conditioned medium was placed in the lower chamber and HUVECs were added in the upper chamber of a modified Boyden chamber. After 24 hours incubation, non-migrating cells were removed and cells migrated through membrane pores (8 μm) were stained with Giemsa and photographed. (A) Micrographs of migrated HUVECs from representative membranes under each experimental condition. a, serum-free DMEM; b, 1 ng/ml bFGF in DMEM; c, WT-hPTTG-CM; d, WT-hPTTG-CM+100 ng/ml anti-bFGF antibody; e, WT-hPTTG-CM+100 ng/ml pre-immune goat IgG; f, M-hPTTG-CM; g, C-CM; h, N-CM.

FIG. 21B shows quantification of migrated cells. Stained cells were extracted with 10% acetic acid and absorbance of extracted solution determined. The data shown is the mean±SD of three separate experiments. D, serum-free DMEM; F, 1 ng/ml of bFGF in DMEM; WT, WT-hPTTG-CM; M, M-hPTTG-CM; C, C-CM; N, N-CM. p<0.01 versus * DMEM, ** WT-hPTTG-CM, *** respective conditioned media (CM) alone.

FIG. 22 demonstrates tube-formation of HUVECs on Matrigel. 5×10⁴ of HUVECs suspended in sample conditioned media and plated on GFR Matrigel thickly coated 24-well culture plates. After 24 hours incubation, cells were photographed under phase-contrast microscopy. FIG. 22A shows micrographs of tube-forming HUVECs. Representative photographs for each experimental condition are shown. a, serum-free DMEM; b, 1 ng/ml bFGF in DMEM; c, WT-hPTTG-CM; d, WT-hPTTG-CM+100 ng/ml anti-bFGF antibody; e, WT-hPTTG-CM+100 ng/ml pre-immune goat IgG; f, M-hPTTG-CM; g, C-CM; h, N-CM. FIG. 22B shows quantification of tube-formation. Tube length was quantified as described in “Materials and Methods”. The mean pixel number±SD of three separate experiments is expressed. From left to right: D, serum-free DMEM; F, 1 ng/ml bFGF in DMEM; WT, WT-hPTTG-CM; M, M-hPTTG-CM; C, C-CM; N, N-CM. p<0.01 versus * DMEM, ** WT-hPTTG-CM, *** respective conditioned medium (CM) alone.

FIG. 23 demonstrates vascular reactions of CAM to conditioned media. Test samples, positive or negative control in collagen sponges were loaded on CAM of 9-day-old chick embryos. After 4 days, CAMs were photographed. (A) photographs of representative CAMs of 13-day-old chick embryo. a, serum-free DMEM; b, 1 ng/ml bFGF in DMEM; c, WT-hPTTG-CM; d, M-hPTTG-CM; e, C-CM; f, N-CM. (B) Quantification of induced vessels. Number of blood vessels entering the collagen sponges was counted under stereomicroscopy. The data shown is the mean±SD of three separate experiments. D, serum-free DMEM; F, 1 ng/ml of bFGF in PBS; WT, WT-hPTTG-CM; M, M-hPTTG-CM; C, C-CM; N, N-CM. p<0.01 versus * PBS, ** WT-hPTTG-CM.

### DESCRIPTION OF THE PREFERRED EMBODIMENTS OF THE INVENTION

The present invention relates to a method of modulating angiogenesis in a tissue comprising mammalian cells, including human cells. The tissue can be malignant tissue, such as a solid tumor malignant neoplasm in any location of the body. Alternatively, the tissue can be non-malignant or normal tissue, such as, but not limited to scar tissue, hepatic tissue, kidney tissue, retinal tissue, cardiovascular tissue, including heart, arteries, veins, and capillaries, or cerebrovascular tissue, including brain tissue and blood vessels of the brain. Thus, among the useful benefits of the method of modulating angiogenesis in a tissue is the ability to inhibit angiogenesis in keloid scar tissue typical of skin tissue healing among bum victims and after wounding of the skin in some populations, such as among African Americans, thus providing a method for limiting scar formation. Conversely, tissue healing and/or tissue regeneration can be promoted by enhancing angiogenesis at the site of wounding or surgical resection.

For the purposes of the inventive methods, the mammalian cell is a cell of human or non-human origin, originating from, or in, any mammalian animal, e.g., a non-human primate, rat, mouse, rabbit, guinea pig, hamster, bovine, porcine, ovine, equine, canine, feline, pachyderm, and the like. The mammalian cell can be situated in vivo, i.e., within a mammalian animal subject or human subject, or in vitro, i.e., the cell can be a cultured cell.

For the purposes of the invention, “neoplastic cellular proliferation” includes neoplastic (malignant or benign), hyperplastic, cytologically dysplastic and/or premalignant cellular growth or proliferation in a mammalian subject or cell culture. Hyperplastic cellular growth or proliferation includes abnormal multiplication or increase in the numbers of normal cells in a normal arrangement in a tissue, for example, as is common in benign prostatic hyperplasia. Cytologically dysplastic and/or premalignant cellular growth or proliferation include increases in cellular numbers of karyotypically abnormal but non-malignant cells within a tissue. Examples include some benign prostatic hyperplasias/dysplasia and cervical hyperplasias/dysplasias.

Neoplastic cellular growth and/or proliferation, i.e., growth of abnormally organized tissue, includes malignant and non-malignant neoplasms. Malignant neoplasms include primary, recurrent, and/or or metastatic cancerous tumors originating in any tissues, for example, carcinomas, sarcomas, lymphomas, mesotheliomas, melanomas, gliomas, nephroblastomas, glioblastomas, oligodendrogliomas, astrocytomas, ependymomas, primitive neuroectodermal tumors, atypical meningiomas, malignant meningiomas, or neuroblastomas, originating in the pituitary, hypothalamus, lung, kidney, adrenal, ureter, bladder, urethra, breast, prostate, testis, skull, brain, spine, thorax, peritoneum, ovary, uterus, stomach, liver, bowel, colon, rectum, bone, lymphatic system, skin, or in any other organ or tissue of the subject.

In accordance with gene-based embodiments of the method of inhibiting neoplastic cellular proliferation and/or transformation or in accordance with the method of modulating angiogenesis in a tissue, directed to inhibiting angiogensis, an inventive composition is delivered to the cell, which composition comprises a PTTG carboxy-terminal-related polynucleotide. A “PTTG carboxy-terminal-related” polynucleotide is a polynucleotide having a contiguous sequence of bases (e.g., adenine [A], thymine [T], uracil [U], guanine [G], and/or cytosine [C]) defining a sequence specific to the 3′ coding region of PTTG. The 3′-end or terminal extends from approximately the mid-point of a cDNA coding sequence encoding a native PTTG to its end at a stop codon. The PTTG carboxy-terminal-related polynucleotide can be a sequence encoding a carboxy-terminal portion of a mammalian PTTG protein (i.e., a PTTG-C peptide), as described more fully below, or encoding a PTTG-specific fragment thereof, or a degenerate coding sequence, or a sequence complementary to any of these.

Alternatively, embodiments of the method of modulating angiogenesis, directed to enhancing angiogenesis (compared to angiogenic activity in the tissue without using the method), involve delivering a PTTG peptide-encoding polynucleotide that encodes a full length PTTG protein or a truncated PTTG that still retains PTTG peptide's biological activity.

In some preferred embodiments, the inventive composition includes a nucleic acid construct, such as a plasmid or viral expression vector, which comprises the polynucleotide in a sense or antisense orientation, and from which PTTG-specific mRNA transcript can be expressed in the cell. In a preferred embodiment, the nucleic acid construct contains a polynucleotide encoding a mammalian PTTG carboxy-terminal (PTTG-C) peptide, which can be any PTTG-C peptide or functional fragment thereof as described herein. The composition can also contain one or more helper plasmids or viruses, if appropriate. The plasmid or viral expression vector is a nucleic acid construct that includes a promoter region operatively linked to the polynucleotide in a transcriptional unit.

As used herein, a promoter region refers to a segment of DNA that controls transcription of a DNA to which it is operatively linked. The promoter region includes specific sequences that are sufficient for RNA polymerase recognition, binding and transcription initiation. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated. Exemplary promoters contemplated for use in the practice of the present invention include the SV40 early promoter, the cytomegalovirus (CMV) promoter, the mouse mammary tumor virus (MMTV) steroid-inducible promoter, Moloney murine leukemia virus (MMLV) promoter, and the like.

As used herein, “expression” refers to the process by which polynucleic acids are transcribed into mRNA and translated into peptides, polypeptides, or proteins. If the polynucleic acid is derived from genomic DNA, expression may, if an appropriate eukaryotic host cell or organism is selected, include splicing of the mRNA.

As used herein, the term “operatively linked” refers to the functional relationship of DNA with regulatory and effector nucleotide sequences, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. Thus, “operatively linked” means that, within a transcriptional unit, the promoter sequence, is located upstream (i.e., 5′ in relation thereto) from the coding sequence and the coding sequence, is 3′ to the promoter, or alternatively is in a sequence of genes or open reading frames 3′ to the promoter and expression is coordinately regulated thereby. Both the promoter and coding sequences are oriented in a 5′ to 3′ manner, such that transcription can take place in vitro in the presence of all essential enzymes, transcription factors, co-factors, activators, and reactants, under favorable physical conditions, e.g., suitable pH and temperature. This does not mean that, in any particular cell, conditions will favor transcription. For example, transcription from a tissue-specific promoter is generally not favored in heterologous cell types from different tissues.

The term “nucleic acid” encompasses ribonucleic acid (RNA) or deoxyribonucleic acid (DNA), which DNA can be complementary DNA (cDNA) or genomic DNA, e.g. a gene encoding a PTTG protein. “Polynucleotides” encompass nucleic acids containing a “backbone” formed by phosphodiester linkages between ribosyl or deoxyribosyl moieties. Polynucleotides also include nucleic acid analogs, for example polynucleotides having alternative linkages as known in the art. Examples include phosphorothioate linkages (e.g., phosphorothioate oligodeoxynucleotides; S-oligonucleotides), mixed phosphorothioate and phosphodiester linkages (e.g., S-O-oligodeoxynucleotides and phosphodiester/phosphorothioate 2′-O-methyl-oligoribonucleotides, Zhou, W. et al., Mixed backbone oligonucleotides as second-generation antisense agents with reduced phosphthioate-related side effects, Bioorg. Med. Chem. Lett. 8(22):3269-74 [1998]), methylphosphonate-phosphodiester modifications (MP-O-oligonucleotides; Zhao, Q. et al., Comparison of cellular binding and uptake of antisense phosphodiester, phosphorothioate, and mixed phosphorothioate and methylphosphonate oligonucleotides, Antisense Res. Dev. 3(1):53-66 [1993]), or morpholino oligonucleotides (e.g., Schmajuk, G. et al., Antisense oligonucleotides with different backbones. Modification of splicing pathways and efficacy of uptake, J. Biol. Chem. 274(31):21783-89 [1999])

Also included among useful polynucleotides are nucleic acid analogs having a pseudopeptide or polyamide backbone comprising N-(2-aminoethyl)glycine moieties, i.e., peptide nucleic acids (PNA). (E.g., Nielsen, P. E., Peptide nucleic acids: on the road to new gene therapeutic drugs, Pharmacol. Toxicol. 86(1):3-7 [2000]; Soomets, U. et al., Antisense properties of peptide nucleic acids, Front. Biosci. 4:D782-86 [1999]; Tyler, B. M. et al., Peptide nucleic acids targeted to the neurotensin receptor and administered i.p. cross the blood-brain barrier and specifically reduce gene expression, Proc. Natl. Acad. Sci. USA 96(12):7053-58 [1999]).

Polynucleotides include sense or antisense polynucleotides. “Polynucleotides” also encompasses “oligonucleotides”.

A polynucleotide sequence complementary to a PTTG-specific polynucleotide sequence, as used herein, is one binding specifically with a PTTG-specific nucleotide base sequence The phrase “binding specifically” encompasses the ability of a polynucleotide sequence to recognize a complementary base sequence and to form double-helical segments therewith via the formation of hydrogen bonds between the complementary base pairs. Thus, a complementary sequence includes, for example, an antisense sequence with respect to a sense sequence or coding sequence.

In some embodiments of the PTTG-encoding or PTTG-C-related polynucleotide, the polynucleotide is in a sense orientation within the transcriptional unit, such that mRNA transcript can be produced, which when translated results in a translation product, such as a PTTG protein or a PTTG carboxy-terminal peptide (PTTG-C). In other embodiments, the PTTG-C-related polynucleotide is in an antisense orientation such that transcription results in a transcript complementary to and hybridizable with a naturally-occurring sense PTTG mRNA molecule under physiological conditions, inhibiting or blocking translation therefrom. Thus, antisense oligonucleotides inactivate target mRNA sequences by either binding thereto and inducing degradation of the mRNA by, for example, RNase I digestion, or inhibiting translation of mRNA target sequence by interfering with the binding of translation-regulating factors or ribosomes, or by inclusion of other chemical structures, such as ribozyme sequences or reactive chemical groups which either degrade or chemically modify the target mRNA. For example, an antisense oligonucleotide targeted to a PTTG carboxy-terminal-related polynucleotide segment of mRNA or genomic DNA is effective in inhibiting expression of PTTG.

Gene-based therapy strategies employing antisense oligonucleotides are well known in the art. (E.g., Rait, A. et al., 3′-End conjugates of minimally phosphorothioate-protected oligonucleotides with 1-O-hexadecylglycerol: synthesis and anti-ras activity in radiation-resistant cells, Bioconjug Chem., 11(2):153-60 [2000]; Stenton, G. R. et al., Aerosolized syk antisense suppresses syk expression, mediator release from macrophages, and pulmonary inflammation, J. Immunol., 164(7):3790-7 [2000]; Suzuki, J. et al., Antisense Bcl-x oligonucleotide induces apoptosis and prevents arterial neointimal formation in murine cardiac allografts, Cardiovas. Res., 45(3):783-7 [2000]; Kim, J. W. et al., Antisense oligodeoxynucleotide of glyceraldehyde-3-phosphate dehyrdogenase gene inhibits cell proliferation and induces apoptosis in human cervical carcinoma cell line, Antisense Nucleic Acid Drug Dev., 9(6):507-13 [1999]; Han, D. C. et al., Therapy with antisense TGF-betal oligodeoxynucleotides reduces kidney weight and matrix mRNAs in diabetic mice, Am. J. Physiol. Renal Physiol., 278(4):F628-F634 [2000]; Scala, S. et al., Adenovirus-mediated suppression of HMGI (Y) protein synthesis as potential therapy of human malignant neoplasias, Proc. Natl. Acad. Sci. USA., 97(8):4256-4261 [2000]; Arteaga, C. L., et al., Tissue-targeted antisense c-fos retroviral vector inhibits established breast cancer xenografts in nude mice, Cancer Res., 56(5):1098-1103 [1996]; Muller, M. et al., Antisense phosphorothioate oligodeoxynucleotide down-regulation of the insulin-like growth factor I receptor in ovarian cancer cells, Int. J. Cancer, 77(4):567-71 [1998]; Engelhard, H. H., Antisense Oligodeoxynucleotide Technology: Potential Use for the Treatment of Malignant Brain Tumors, Cancer Control, 5(2): 163-170 [1998]; Alvarez-Salas, L. M. et al., Growth inhibition of cervical tumor cells by antisense oligodeoxynucleotides directed to the human papillomavirus type 16 E6 gene, Antisense Nucleic Acid Drug Dev., 9(5):441-50 [1999]; Im, S A., et al., Antiangiogenesis treatment for gliomas: transfer of antisense-vascular endothelial growth factor inhibits tumor growth in vivo, Cancer Res., 59(4):895-900 [1999]; Maeshima, Y. et al., Antisense oligonucleotides to proliferating cell nuclear antigen and Ki-67 inhibit human mesangial cell proliferation, J. Am. Soc. Nephrol., 7(10):2219-29 [1996]; Chen, D. S et al., Retroviral Vector-mediated transfer of an antisense cyclin G1 construct inhibits osteosarcoma tumor growth in nude mice, Hum. Gene Ther, 8(14):1667-74 [1997]; Hirao, T et al., Antisense epidermal growth factor receptor delivered by adenoviral vector blocks tumor growth in human gastric cancer, Cancer Gene Ther. 6(5):423-7 [1999]; Wang, X. Y. et al., Antisense inhibition of protein kinase Calpha reverses the transformed phenotype in human lung carcinoma cells, Exp. Cell Res., 250(1):253-63 [1999]; Sacco, M. G. et al., In vitro and in vivo antisense-mediated growth inhibition of a mammary adenocarcinoma from MMTV-neu transgenic mice, Gene Ther., 5(3);388-93 [1998]; Leonetti, C. et al., Antitumor effect of c-myc antisense phosphorothioate oligodeoxynucleotides on human melanoma cells in vitro and in mice, J. Natl. Cancer Inst., 88(7):419-29 [1996]; Laird, A. D. et al., Inhibition of tumor growth in liver epthelial cells transfected with a transforming growth factor alpha antisense gene, Cancer Res. 54(15):4224-32 (Aug. 1, 1994); Yazaki, T. et al., Treatment of glioblastonia U-87 by systemic administration of an antisense protein kinase C-alpha phosphorothioate oligodeoxynucleotide, Mol. Pharmacol., 50(2):236-42 [1996]; Ho, P. T. et al., Antisense oligonucleotides as therapeutics for malignant diseases, Semin. Oncol, 24(2):187-202 [1997]; Muller, M. et al., Antisense phosphorothioate oligodeoxynucleotide down-regulation of the insulin-like growth factor I receptor in ovarian cancer cells, Int. J. Cancer, 77(4):567-71 [1998]; Elez, R. et al., Polo-like kinasel, a new target for antisense tumor therapy, Biochem. Biophys. Res Commun., 269(2):352-6 [2000]; Monia, B. P. et al., Antitumor activity of aphosphorothioate antisense oligodeoxynucleotide targeted against C-raf kinase, Nat. Med., 2(6):668-75 [1996]).

In other embodiments of the inventive method, the inventive composition comprises a PTTG-encoding or PTTG carboxy-terminal-related polynucleotide that is not contained in an expression vector, for example, a synthetic antisense oligonucleotide, such as a phosphorothioate oligodeoxynucleotide. Synthetic antisense oligonucleotides, or other antisense chemical structures designed to recognize and selectively bind to mRNA, are constructed to be complementary to portions of the PTTG coding strand, for example, to coding sequences shown in SEQ ID NOS:1, 3, 10, 15, 18, or 19 (Tables 1-6 below). By preventing translational expression of at least part of the PTTG 3′ coding region, an antisense PTTG carboxy-terminal-related polynucleotide is useful, in accordance with the inventive method, to prevent expression of PTTG protein that is functional in mediating neoplastic cellular proliferation and/or transformation.

In preferred embodiments of the method of inhibiting neoplastic cellular proliferation and/or transformation, the composition also comprises an uptake-enhancing agent as further described herein. Inventive compositions, containing the uptake-enhancing agent complexed with a PTTG-specific polynucleotide, are designed to be capable of passing through the cell membrane in order to enter the cytoplasm of the cell by virtue of physical and chemical properties. In addition, the composition can be designed for delivery only to certain selected cell populations by targeting the composition to be recognized by specific cellular uptake mechanisms which take up the PTTG-specific polynucleotides only within select cell populations. For example, the composition can include a receptor agonist to bind to a receptor found only in a certain cell type.

The inventive composition can also optionally contain one or more pharmaceutically acceptable carrier(s). As used herein, the term “acceptable carrier” encompasses any of the standard pharmaceutical carriers. The carrier can be an organic or inorganic carrier or excipient, such as water and emulsions such as an oil/water or water/oil emulsion, and various types of wetting agents. The active ingredient(s) can optionally be compounded in a composition formulated, for example, with non-toxic, pharmaceutically acceptable carriers for infusions, tablets, pellets, capsules, solutions, emulsions, suspensions, and any other form suitable for use Such carriers also include glucose, lactose, gum acacia, gelatin, mannitol, starch paste, magnesium trisilicate, talc, corn starch, keratin, colloidal silica, potato starch, urea, medium chain length triglycerides, dextrans, normal saline, phosphate buffered saline and other carriers suitable for use in manufacturing preparations, in solid, semisolid, or liquid form. In addition auxiliary, stabilizing, thickening and coloring agents and perfumes can be used as appropriate.

PTTG-specific polynucleotides, including PTTG-encoding and PTTG carboxy-terminal-related polynucleotides, are determined by base sequence similarity or homology to known mammalian PTTG-specific nucleotide sequences. Base sequence homology is determined by conducting a base sequence similarity search of a genomics data base, such as the GenBank database of the National Center for Biotechnology Information (NCBI; www.ncbi.nlm.nih.gov/BLAST/), using a computerized algorithm, such as PowerBLAST, QBLAST, PSI-BLAST, PHI-BLAST, gapped or ungapped BLAST, or the “Align” program through the Baylor College of Medicine server (www.hgsc.bcm.tmc.edu/seq_data). (E.g., Altchul, S. F., et al., Gapped BLAST and PSI-BLAST: a new generation of protein database search programs, Nucleic Acids Res. 25(17):3389-402 [1997]; Zhang, J., & Madden, T. L., PowerBLAST: a new network BLAST application for interactive or automated sequence analysis and annotation, Genome Res. 7(6):649-56 [1997]; Madden, T. L., et al., Applications of network BLAST server, Methods Enzymol. 266:131-41 [1996]; Altschul, S. F., et al., Basic local alignment search tool, J. Mol. Biol. 215(3):403-10 [1990]). Preferably, a PTTG-specific polynucleotide sequence is at least 5 to 30 contiguous nucleotides long, more preferably at least 6 to 15 contiguous nucleotides long, and most preferably at least 7 to 10 contiguous nucleotides long. Preferably, the inventive PTTG carboxy-terminal-related polynucleotide is at least about 45 contiguous nucleotides long.

Preferred examples of PTTG-specific coding sequences include the sequence for human PTTG (hPTTG or PTTG1). The PTTG1 peptide is encoded by the open reading frame at nucleotide positions 95 through 700 of human PTTG1 gene sequence SEQ. ID. NO.:3 (Table 1 below).

TABLE 1
PTTG1 gene sequence.

| | |
|---|---|
| 1 | ATGGCCGCGA GTTGTGGTTT AAACCAGGAG TGCCGCGCGT CCGTTCACCG |
| 51 | CGGCCTCAGA TGAATGCGGC TGTTAAGACC TGCAATAATC CAGAATGGCT |
| 101 | ACTCTGATCT ATGTTGATAA GGAAAATGGA GAACCAGGCA CCCGTGTGGT |
| 151 | TGCTAAGGAT GGGCTGAAGC TGGGGTCTGG ACCTTCAATC AAAGCCTTAG |
| 201 | ATGGGAGATC TCAAGTTTCA ACACCACGTT TTGGCAAAAC GTTCGATGCC |
| 251 | CCACCAGCCT TACCTAAAGC TACTAGAAAG GCTTTGGGAA CTGTCAACAG |
| 301 | AGCTACAGAA AAGTCTGTAA AGACCAAGGG ACCCCTCAAA CAAAAACAGC |
| 351 | CAAGCTTTTC TGCCAAAAAG ATGACTGAGA AGACTGTTAA AGCAAAAAGC |
| 401 | TCTGTTCCTG CCTCAGATGA TGCCTATCCA GAAATAGAAA AATTCTTTCC |
| 451 | CTTCAATCCT CTAGACTTTG AGAGTTTTGA CCTGCCTGAA GAGCACCAGA |
| 501 | TTGCGCACCT CCCCTTGAGT GGAGTGCCTC TCATGATCCT TGACGAGGAG |
| 551 | AGAGAGCTTG AAAAGCTGTT TCAGCTGGGC CCCCCTTCAC CTGTGAAGAT |
| 601 | GCCCTCTCCA CCATGGGAAT CCAATCTGTT GCAGTCTCCT TCAAGCATTC |
| 651 | TGTCGACCCT GGATGTTGAA TTGCCACCTG TTTGCTGTGA CATAGATATT |
| 701 | TAAATTTCTT AGTGCTTCAG AGTTTGTGTG TATTTGTATT AATAAAGCAT |
| 751 | TCTTTAACAG ATAAAAAAAA AAAAAAAAA (SEQ. ID. NO.:3). |

The 3′ coding region of PTTG1 includes the following 168-nucleotide sequence, which corresponds to nucleotide positions 533 through 700 of SEQ. ID. NO.:3, shown in Table 2 below.

TABLE 2
Portion of 3′ coding region of PTTG1

| | |
|---|---|
| 1 | ATGATCCTTG ACGAGGAGAG AGAGCTTGAA AAGCTGTTTC AGCTGGGCCC |
| 51 | CCCTTCACCT GTGAAGATGC CCTCTCCACC ATGGGAATCC AATCTGTTGC |
| 101 | AGTCTCCTTC AAGCATTCTG TCGACCCTGG ATGTTGAATT GCCACCTGTT |
| 151 | TGCTGTGACA TAGATATT (SEQ. ID. NO:10). |

Another useful example of a PTTG-specific coding sequence is a sequence that encodes a rat PTTG peptide, including nucleotide positions 293 through 889 of SEQ. ID. NO.:1 (Table 3 below).

TABLE 3
Rat PTTG sequence.

| | | |
|---|---|---|
| AATTCGGCAC GAGCCAACCT TGAGCATCTG ATCCTCTTGG CTTCTCCTTC CTATCGCTGA | 60 | (SEQ. ID. NO.:1) |
| GCTGGTAGGC TGGAGACAGT TGTTTGGGTG CCAACATCAA CAAACGATTT CTGTAGTTTA | 120 | |
| GCGTTTATGA CCCTGGCGTG AAGATTTAAG GTCTGGATTA AGCCTGTTGA CTTCTCCAGC | 180 | |
| TACTTCTAAA TTTTTGTGCA TAGGTGCTCT GGTCTCTGTT GCTGCTTAGT TCTTCCAGCC | 240 | |
| TTCCTCAATG CCAGTTTTAT AATATGCAGG TCTCTCCCCT CAGTAATCCA GG ATG | 295 | |
| GCT ACT CTG ATC TTT GTT GAT AAG GAT AAC GAA GAG CCA GGC AGC CGT | 343 | |
| TTG GCA TCT AAG GAT GGA TTG AAG CTG GGC TCT GGT GTC AAA GCC TTA | 391 | |
| GAT GGG AAA TTG CAG GTT TCA ACG CCA CGA GTC GGC AAA GTG TTC GGT | 439 | |
| GCC CCA GGC TTG CCT AAA GCC AGC AGG AAG GCT CTG GGA ACT GTC AAC | 487 | |
| AGA GTT ACT GAA AAG CCA GTG AAG AGT AGT AAA CCC CTG CAA TCG AAA | 535 | |
| CAG CCG ACT CTG AGT GTG AAA AAG ATC ACC GAG AAG TCT ACT AAG ACA | 583 | |
| CAA GGC TCT GCT CCT GCT CCT GAT GAT GCC TAC CCA GAA ATA GAA AAG | 631 | |
| TTC TTC CCC TTC GAT CCT CTA GAT TTT GAG AGT TTT GAC CTG CCT GAA | 679 | |
| GAG CAC CAG ATC TCA CTT CTC CCC TTG AAT GGA GTG CCT CTC ATG ATC | 727 | |
| CTG AAT GAA GAG AGG GGG CTT GAG AAG CTG CTG CAC CTG GAC CCC CCT | 775 | |
| TCC CCT CTG CAG AAG CCC TTC CTA CCG TGG GAA TCT GAT CCG TTG CCG | 823 | |
| TCT CCT CCC AGC GCC CTC TCC GCT CTG GAT GTT GAA TTG CCG CCT GTT | 871 | |
| TGT TAC GAT GCA GAT ATT TAAACGTCTT ACTCCTTTAT AGTTTATGTA | 919 | |
| AGTTGTATTA ATAAAGCATT TGTGTGTAAA AAAAAAAAAA AAAACTCGAG AGTAC | 974 | |

The 3′ coding region of rat PTTG includes the following 168-nucleotide sequence, which corresponds to nucleotide positions 722 through 889 of SEQ. ID. NO.:1, shown in Table 4 below.

TABLE 4
Portion of 3′ coding region of rat PTTG.

| | |
|---|---|
| ATG ATC CTG AAT GAA GAG AGG GGG CTT GAG AAG CTG CTG CAC CTG GAC | 48 |
| CCC CCT TCC CCT CTG CAG AAG CCC TTC CTA CCG TGG GAA TCT GAT CCG | 96 |
| TTG CCG TCT CCT CCC AGC GCC CTC TCC GCT CTG GAT GTT GAA TTG CCG | 144 |
| CCT GTT TGT TAC GAT GCA GAT ATT (SEQ. ID. NO:18). | 168 |

Another useful example of a PTTG-specific coding sequence is a sequence that encodes a murine PTTG peptide, including nucleotide positions 304 through 891 of SEQ. ID. NO.:15 (Table 5 below).

TABLE 5
Murine PTTG sequence.

| | |
|---|---|
| 1 | TCTTGAACTT GTTATGTAGC AGGAGGCCAA ATTTGAGCAT CCTCTTGGCT TCTCTTTATA |
| 61 | GCAGAGATTG TAGGCTGGAG ACAGTTTTGA TGGGTGCCAA CATAAACTGA TTTCTGTAAG |
| 121 | AGTTGAGTGT TTTATGACCC TGGCGTGCAG ATTTAGGATC TGGATTAAGC CTGTTGACTT |
| 181 | CTCCAGCTAC TTATAAATTT TTGTGCATAG GTGCCCTGGG TAAAGCTTGG TCTCTGTTAC |
| 241 | TGCGTAGTTT TTCCAGCCGT CTCAATGCCA ATATTCAGGC TCTCTCCCTT AGAGTAATCC |
| 301 | AGAATGGCTA CTCTTATCTT TGTTGATAAG GATAATGAAG AACCCGGCCG CCGTTTGGCA |
| 361 | TCTAAGGATG GGTTGAAGCT GGGCACTGGT GTCAAGGCCT TAGATGGGAA ATTGCAGGTT |
| 421 | TCAACGCCTC GAGTCGGCAA AGTGTTCAAT GCTCCAGCCG TGCCTAAAGC CAGCAGAAAG |
| 481 | GCTTTGGGGA CAGTCAACAG AGTTGCCGAA AAGCCTATGA AGACTGGCAA ACCCCTCCAA |
| 541 | CCAAAACAGC CGACCTTGAC TGGGAAAAAG ATCACCGAGA AGTCTACTAA GACACAAAGC |
| 601 | TCTGTTCCTG CTCCTGATGA TGCCTACCCA GAAATAGAAA AGTTCTTCCC TTTCAATCCT |
| 661 | CTAGATTTTG ACCTGCCTGA GGAGCACCAG ATCTCACTTC TCCCCTTGAA TGGCGTGCCT |
| 721 | CTCATCACCC TGAATGAAGA GAGAGGGCTG GAGAAGCTGC TGCATCTGGG CCCCCCTAGC |
| 781 | CCTCTGAAGA CACCCTTTCT ATCATGGGAA TCTGATCCGC TGTACTCTCC TCCCAGTGCC |
| 841 | CTCTCCACTC TGGATGTTGA ATTGCCGCCT GTTTGTTACG ATGCAGATAT TTAAACTTCT |
| 901 | TACTTCTTTG TAGTTTCTGT ATGTATGTTG TATTAATAAA GCATT. (SEQ. ID. NO:15) |

The 3′ coding region of murine PTTG includes the following 168-nucleotide sequence, which corresponds to nucleotide positions 724 through 891 of SEQ. ID. NO.:15, shown in Table 6 below.

TABLE 6
Portion of 3′ coding region of murine PTTG.

| | |
|---|---|
| ATCACCCTGA ATGAAGAGAG AGGGCTGGAG AAGCTGCTGC ATCTGGGCCC CCCTAGCCCT | 60 |
| CTGAAGACAC CCTTTCTATC ATGGGAATCT GATCCGCTGT ACTCTCCTCC CAGTGCCCTC | 120 |
| TCCACTCTGG ATGTTGAATT GCCGCCTGTT TGTTACGATG CAGATATT | 168 |
| (SEQ. ID. NO.:19). | |

Inventive PTTG-C-related polynucleotides having nucleotides sequences of SEQ. ID. NOS.:10, 18, or 19, degenerate coding sequences, or sequences complementary to any of these, are merely illustrative of useful PTTG carboxy-terminal-related polynucleotides. Other useful PTTG carboxy-terminal-related polynucleotides are functional fragments of any of SEQ. ID. NOS.:10, 18, or 19 at least about 45 contiguous nucleotides long, degenerate coding sequences, or sequences complementary to any of these, the presence of which in the cell can function to downregulate endogenous PTTG expression and/or PTTG function, which functionality can be determined by routine screening.

As used herein, the term “degenerate” refers to codons that differ in at least one nucleotide from a reference nucleic acid, e.g., SEQ ID NOS:1, 3, 10, 15, 18, or 19, but encode the same amino acids as the reference nucleic acid. For example, codons specified by the triplets “UCU”, “UCC”, “UCA”, and “UCG” are degenerate with respect to each other since all four of these codons encode the amino acid serine.

Other useful polynucleotides include nucleic acids or other polynucleotides, that differ in sequence from the sequences shown in SEQ ID NO:1, SEQ. ID. NO.:3, SEQ. ID. NO.:10, SEQ. ID. NO:15, SEQ. ID. NO.:18, and SEQ. ID. NO.:19, but which when expressed in a cell, result in the same phenotype. Phenotypically similar nucleic acids are also referred to as “functionally equivalent nucleic acids”. As used herein, the phrase “functionally equivalent nucleic acids” encompasses nucleic acids characterized by slight and non-consequential sequence variations that will function in substantially the same manner, compared to any of the detailed nucleotide sequences disclosed herein, to produce PTTG protein functional with respect to inducing neoplastic cellular proliferation and/or transformation or as to inducing or enhancing angiogenesis, or PTTG-C peptide(s) functional with respect to inhibition of neoplastic cellular proliferation and/or transformation or inhibiting angiogenesis, and/or polypeptide products functional with respect to immunogenicity. Such polynucleotides can have substantially the same coding sequences as the reference sequences, encoding the amino acid sequence as set forth in SEQ. ID. NO.:2, SEQ. ID. NO.:4, SEQ. ID. NO.:9, SEQ. ID. NO:14, SEQ. ID. NO.:16, or SEQ. ID. NO.:17 or a larger amino acid sequence including SEQ. ID. NO.:2, SEQ. ID. NO.:4, SEQ. ID. NO.:9, SEQ. ID. NO.:14, SEQ. ID. NO.:16, or SEQ. ID NO.:17. As employed herein, the term “substantially the same nucleotide sequence” refers to DNA having sufficient identity to the reference polynucleotide, such that it will hybridize to the reference nucleotide under moderately stringent hybridization conditions. In other embodiments, DNA having “substantially the same nucleotide sequence” as the reference nucleotide sequence has at least about 60% identity with respect to the reference nucleotide sequence. DNA having at least 70%, more preferably at least 90%, yet more preferably at least 95%, identity to the reference nucleotide sequence is preferred.

In preferred embodiments, functionally equivalent nucleic acids encode polypeptides or peptide fragments that are the same as those disclosed herein or that have conservative amino acid variations, or that encode larger polypeptides that include SEQ. ID. NO.:2, SEQ. ID. NO.:4, SEQ. ID. NO.:9, or SEQ. ID. NO.:14, SEQ. ID. NO.:16, or SEQ. ID. NO.:17, or fragments of any of these that are biologically functional fragments with respect to inhibiting neoplastic cellular proliferation and/or transformation or inhibiting angiogenesis. For example, conservative variations include substitution of a non-polar residue with another non-polar residue, or substitution of a charged residue with a similarly charged residue. These variations include those recognized by skilled artisans as those that do not substantially alter the tertiary structure of the protein.

Useful polynucleotides can be produced by a variety of methods well-known in the art, e g., by employing PCR and other similar amplification techniques, using oligonucleotide primers specific to various regions of SEQ ID NOS:1, 3, 10, 15, 18, 19, or functionally equivalent polynucleotide sequences. Other synthetic methods for producing polynucleotides or oligonucleotides of various lengths are also well known.

In accordance with the method, preferred polynucleotides hybridize under moderately stringent, preferably high stringency, conditions to substantially the entire sequence, or substantial portions (i.e., typically at least 15-30 nucleotide) of the nucleic acid sequence set forth in SEQ ID NOS:1, 3, 10, 15, 18, or 19, or to complementary sequences

The phrase “stringent hybridization” is used herein to refer to conditions under which annealed hybrids, or at least partially annealed hybrids, of polynucleic acids or other polynucleotides are stable. As known to those of skill in the art, the stability of hybrids is reflected in the melting temperature (Tₘ) of the hybrids. In general, the stability of a hybrid is a function of sodium ion concentration and temperature. Typically, the hybridization reaction is performed under conditions of relatively low stringency, followed by washes of varying, but higher, stringency. Reference to hybridization stringency relates to such washing conditions.

As used herein, the phrase “moderately stringent hybridization” refers to conditions that permit target-DNA to bind a complementary nucleic acid that has about 60% sequence identity or homology, preferably about 75% identity, more preferably about 85% identity to the target DNA; with greater than about 90% identity to target-DNA being especially preferred. Preferably, moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 65° C.

The phrase “high stringency hybridization” refers to conditions that permit hybridization of only those nucleic acid sequences that form stable hybrids in 0.018 M NaCl at 65° C. (i.e., if a hybrid is not stable in 0.018 M NaCl at 65° C., it will not be stable under high stringency conditions, as contemplated herein). High stringency conditions can be provided, for example, by hybridization in 50% formamide, 5×Denhart's solution, 5×SSPE, 0.2% SDS at 42° C., followed by washing in 0.1×SSPE, and 0.1% SDS at 65° C.

The phrase “low stringency hybridization” refers to conditions equivalent to hybridization in 10% formamide, 5×Denhart's solution, 6×SSPE, 0.2% SDS at 42° C., followed by washing in 1×SSPE, 0.2% SDS, at 50° C. Denhart's solution and SSPE (see, e.g., Sambrook et al., Molecular Cloning, A Laboratory Manual, Cold Spring Harbor Laboratory Press [1989]) are well known to those of skill in the art as are other suitable hybridization buffers.

The PTTG-encoding or PTTG carboxy-terminal-related polynucleotide can be, but is not necessarily, of homologous origin with respect to the cell, due to the relatively high degree of sequence homology among mammalian PTTG sequences. PTTG-encoding or PTTG carboxy-terminal-related polynucleotides of heterologous mammalian origin with respect to the cell is also useful. Thus, for example, in accordance with the inventive method, a human PTTG-C-encoding sequence functions to down regulate endogenous PTTG expression and/or PTTG function in cells of non-human mammalian origin, such as murine or rat cells, and vice versa.

In preferred embodiments of the method of inhibiting neoplastic cellular proliferation and/or transformation of a mammalian cell or the method of modulating angiogenesis in a tissue, the polynucleotide is complexed with a cellular uptake-enhancing agent, in an amount and under conditions sufficient to enter the cell. An “uptake-enhancing” agent, as utilized herein, means a composition of matter for enhancing the uptake of exogenous polynucleotides, such as DNA segment(s), nucleic acid analogs, or nucleic acid constructs, into a eukaryotic cell, preferably a mammalian cell, and more preferably a human cell. The enhancement is measured relative to the polynucleotide uptake in the absence of the uptake-enhancing agent, in the process of transfecting or transducing the cell. Complexation with uptake-enhancing agent(s) generally augments the uptake of a polynucleotide into the cell and/or reduces its breakdown by nucleases during its passage through the cytoplasm.

In accordance with preferred embodiments of the inventive method, PTTG-encoding polynucleotides, PTTG carboxy-terminal-related polynucleotides, PTTG peptides or PTTG-C peptides are complexed with an uptake-enhancing agent. “Complexed” means that the polynucleotide or peptide is a constituent or member of a complex, mixture, or adduct resulting from chemical binding or bonding between and/or among the other constituents, including the cellular uptake-enhancing agent(s), and/or their moieties. Chemical binding or bonding can have the nature of a covalent bond, ionic bond, hydrogen bond, hydrophobic bond, or any combination of these bonding types linking the constituents of the complex at any of their parts or moieties, of which a constituent can have one or a multiplicity of moieties of various sorts. Not every constituent of a complex need be bound to every other constituent, but each constituent has at least one chemical bond with at least one other constituent of the complex. Constituents can include, but are not limited to, molecular compounds of a polar, non-polar, or detergent character; ions, including cations, such as, but not limited to, Na⁺, K⁺, Li⁻, Ca²⁺, Mg²⁺, Fe²⁺, Fe³⁺, Zn²⁺, Cu⁺, Cu²⁺, and/or NH₄⁺, or anions, such as, but not limited to Cl⁻, Br⁻, Fl⁻, NO₃⁻, NO₂⁻, NO⁻, HCO₃⁻, CO₃²⁻, SO₄²⁻, and/or PO₄³⁻; biological molecules, such as proteins, oligopeptides, polypeptides, oligonucleotides, nucleic acids, nucleic acid constructs, plasmids, viral particles, an/or organic polymers and co-polymers.

PTTG-encoding polynucleotides, PTTG carboxy-terminal-related polynucleotides, PTTG peptides, or PTTG-C peptides can be, but are not necessarily, directly bound to the cellular uptake-enhancing agent. For example, the polynucleotide can be contained in an expression vector or other nucleic acid construct, which vector or other construct is bound to the uptake-enhancing agent at some moiety or part of he vector or construct not directly linked to the polynucleotide; for purposes of the present invention, the polynucleotide is still “complexed” with the uptake-enhancing agent, although not being directly bound to the uptake-enhancing agent by a chemical bond. As long as the polynucleotide and the uptake enhancing agent are both constituents or members of the same complex, an indirect chemical linkage suffices. An example with respect to PTTG or PTTG-C peptides, is an intervening third peptide sequence linking a first PTTG or PTTG-C peptide segment with a second cell uptake-enhancing and/or importation-competent peptide segment. The first and second peptide segments, indirectly linked, are “complexed” for purposes of the invention.

Examples of uptake-enhancing agents usefully complexed with the polynucleotide include cationic or polycationic lipid-DNA or liposome-DNA complexes (“lipoplexes”). Such lipoplexes can, optionally, also be coated with serum albumin or formulated as large-sized colloidally unstable complexes to further enhance transfection efficiency; the presence of calcium di-cations (Ca²⁺) can also enhance lipid-based transfection efficiency. (E.g., Simoes, S. et al., Human serum albumin enhances DNA transfection by lipoplexes and confers resistance to inhibition by serum, Biochim. Biophys. Acta 1463(2).459-69 [2000]; Turek, J. et al., Formulations which increase the size of lipoplexes prevent serum-associated inhibition of transfection, J. Gene Med. 2(1):32-40 [2000]; Zudam, N. J. et al., Lamellarity of cationic liposomes and mode of preparation of lipoplexes affect transfection efficiency, Biochim. Biophys. Acta 1419(2):207-20 [1999]; Lam, A. M. and Cullis, P. R., Calcium enhances the transfection potency of plasmid DNA-cationic liposome complexes, Biochim. Biophys. Acta 1463(2):279-290 [2000]).

Inventive compositions can include negatively charged ternary complexes of cationic liposomes, transferrin or fusigenic peptide(s)or poly(ethylenimine). (E.g., Simoes, S. et al., Gene delivery by negatively charged ternary complexes of DNA, cationic liposomes and transferrin or fusigenic peptides, Gene Ther. 5(7):955-64 [1998]). Liposomal uptake-enhancing agents complexed with inventive polynucleotide(s) can also be encapsulated in polyethylene glycol (PEG), FuGENE6, or the like. (E.g., Saravolac, E. G., et al, Encapsulation of plasmid DNA in stabilized plasmid-lipid particles composed of different cationic lipid concentration for optimal transfection activity, J. Drug Target 7(6):423-37 [2000]; Yu, R. Z. et al., Pharmacokinetics and tissue disposition in monkeys of an antisense oligonucleotide inhibitor of Ha-ras encapsulated in stealth liposomes, Pharm. Res. 16(8):1309-15 [1999], Tao, M. et al., Specific inhibition of human telomerase activity by transfection reagent, FuGENE6-antisense phophorothioate oligonucleotide complex in HeLa cells, FEBS Lett 454(3):312-6 [1999]).

In some embodiments, the uptake of antisense oligonucleotides is also enhanced by complexation with biocompatible polymeric or co-polymeric nanoparticles, for example, comprising alginate, aminoalkylmethacrylate, methylmethacrylate, polymethylmethacrylate, methylaminoethyl-methacrylate, polyalkylcyanoacrylate (e.g., polyhexylcyanoacrylate), or the like. (E.g., Aynie, I. et al, Spongelike alginate nanoparticles as a new potential system for the delivery of antisense oligonucleotides, Antisense Nucleic Acid Drug Dev. 9(3):301-12 [1999]; Zimmer, A., Antisense oligonucleotide delivery with polyhexylcyanoacrylate nanoparticles as carriers, Methods 18(3):286-95, 322 [1999]; Berton, M. et al., Highly loaded nanoparticulate carrier using an hydrophobic antisense oligonucleotide complex, Eur. J. Pharm. Sci. 9(2):163-70 [1999]; Zobel, H. P. et al., Evaluation of aminoalkylmethacrylate nanoparticles as colloidal drug carrier systems. Part II: characterization of antisense oligonucleotides loaded copolymer nanoparticles, Eur. J. Pharm. Biopharm. 48(1):1-12 [1999]; Fattal, E. et al., Biodegradable polyalkylcyanoacrylate nanoparticles for the delivery of oligonucleotides, J. Controlled Release 53(1-3):137-43 [1998]).

Other useful uptake-enhancing agents for complexing with polynucleotides include starburst polyamidoamine (PAMAM) dendrimers. (E.g., Yoo, H. et al., PAMAM dendrimers as delivery agents for antisense oligonucleotides, Pharm. Res. 16(12):1799-804 [1999]; Bielinska, A. U. et al., Application of membrane-based dendrimer/DNA complexes for solid phase transfection in vitro and in vivo, Biomaterials 21(9):877-87 [2000]; Bielinska, A. U. et al., DNA complexing with polyamidoamine dendrimers: implications for transfection, Bioconjug. Chem. 10(5):843-50 [1999]; Bielinska, A. U. et al., Regulation of in vitro gene expression using antisense oligonucleotides or antisense expression plasmids transfected using starburst PAMAM dendrimers, Nucleic Acid Res. 24(11):2176-82 [1996]; Kukowska-Latallo, J. F. et al., Efficient transfer of genetic material into mammalian cells using Starburst polyamidoamine dendrimers, Proc. Natl. Acad. Sci. USA 93(10):4897-902 [1996]; Delong, R. et al., Characterization of complexes of oligonucleotides with polyamidoamine starburst dendrimers and effects on intracellular delivery, J. Pharm. Sci. 86(6):762-64 [1997]).

Other preferred uptake-enhancing agents include lipofectin, lipfectamine, DIMRIE C, Superfect, Effectin (Qiagen), unifectin, maxifectin, DOTMA, DOGS (Transfectam, dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, or poly(ethylenimine) (PEI), and/or peptides, such as polylysine, protamine, pK17, peptide K8, and peptide p2. (E.g., Ferkol, Jr. et al., U.S. Pat. Nos., 5,972,900 and 5,972,901; Vaysse, L. and Arveiler, B., Transfection using synthetic peptides: comparison of three DNA-compacting peptides and effect of centrifugation, Biochim. Biophys. Acta 1474(2):244-50 [2000]; Ni, Y. H. et al., Protamine enhance the efficiency of liposome-mediated gene transfer in a cultured human hepatoma cell line, J. Formos. Med Assoc. 98(8):562-66 [1999]; Banerjee, R. et al., Novel series of non-glycerol-based cationic transfection lipids for use in liposomal gene delivery, J. Med. Chem. 42(21):4292-99 [1999]; Godbey, W. T. et al., Improved packing of poly(ethylenimine/DNA complexes increases transfection efficiency, Gene Ther. 6(8): 1380-88 [1999]; Kichler, A et al., Influence of the DNA complexation medium on the transfection efficiency of lipospermine/DNA particles, Gene Ther. 5(6):855-60 [1998]; Birchaa, J. C. et al., Physico-chemical characterisation and transfection efficiency of lipid-based gene delivery complexes, Int. J. Pharm. 183(2): 195-207 [1999]). These non-viral cellular uptake-enhancing agents have the advantage that they facilitate stable integration of xenogeneic DNA sequences into the vertebrate genome, without size restrictions commonly associated with virus-derived transfecting or transducing agents.

Another example, a viral cellular uptake-enhancing agent, is the adenovirus enhanced transferrin-polylysine-mediated gene delivery system has been described and patented by Curiel et al. (Curiel D. T.,et al., Adenovirus enhancement of transferrin-polylysine-mediated gene delivery, PNAS USA 88: 8850-8854 (1991). The delivery of DNA depends upon endocytosis mediated by the transferrin receptor (Wagner et al., Transferrin-polycation conjugates as carriers for DNA uptake into cells, PNAS (USA) 87: 3410-3414 (1990). In addition this method relies on the capacity of adenoviruses to disrupt cell vesicles, such as endosomes and release the contents entrapped therein. This system can enhance the gene delivery to mammalian cells by as much as 2,000 fold over other methods.

A particularly useful uptake enhancing agent for delivery of polynucleotides to human T-lymphocytes is a “tamed” (i.e., lacking native viral genes required for immunodeficiency pathogenesis and/or virulence) Human Immunodeficiency Virus (HIV) vector, which mediates uptake via the CD4 molecule. A variety of such tamed viral vectors, binding with CD4 and/or with other T-lymphocyte markers, are also known in the art.

The amount of each component of the composition is chosen so that the gene modification, e.g., by transfection or transduction, of a mammalian cell is optimized. Such optimization requires no more than routine experimentation. The ratio of polynucleotide to lipid is broad, preferably about 1:1, although other effective proportions can also be utilized depending on the type of lipid uptake-enhancing agent and polynucleotide utilized. (E.g., Banerjee, R. et al. [1999]; Jaaskelainen, I. et al., A lipid carrier with a membrane active component and a small complex size are required for efficient cellular delivery of anti-sense phosphorothioate oligonucleotides, Eur. J. Pharm. Sci. 10(3):187-193 [2000]; Sakurai, F. et al, Effect of DNA/liposome mixing ratio on the physicochemical characteristics, cellular uptake and intracellular trafficking of plasmid DNA/cationic liposome complexes and subsequent gene expression, J. Controlled Release 66(2-3):255-69 [2000]).

A suitable amount of the inventive polynucleotide to be delivered to the cells, in accordance with the method, preferably ranges from about 0.1 nanograms to about 1 milligram per gram of tumor tissue, in vivo, or about 0.1 nanograms to about 1 microgram per 5000 cells, in vitro. Suitable amounts for particular varieties of PTTG-C-related polynucleotides and/or cell types and/or for various mammalian subjects undergoing treatment, can be determined by routine experimentation. For example, malignant cell lines, such as MCF-7 or HeLa, typically are more efficiently transfected by the inventive PTTG-C-related polynucleotides than non-malignant cell lines. Also, those skilled in the art are aware that there is typically considerable variability among individual cancer patients to any single treatment regimen, therefore, the practitioner will tailor any embodiment of the inventive method to each individual patient as appropriate.

In some preferred embodiments, the polynucleotide can be delivered into the mammalian cell, either in vivo or in vitro using suitable expression vectors well-known in the art ( e.g., retroviral vectors, such as lentiviral vectors, or adenovirus vectors, and the like). (See, e.g., Anderson, W. F., Gene therapy scores against cancer, Nat. Med 6(8):862-63 [2000]). In addition, to inhibit the in vivo expression of PTTG, the introduction by expression vector of the antisense strand of a DNA encoding a PTTG-C peptide is contemplated.

Suitable expression vectors are well-known in the art, and include vectors capable of expressing DNA operatively linked to a regulatory sequence, such as a promoter region that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors are well known to those of skill in the art and include those that are replicable in eukaryotic cells and/or prokaryotic cells and those that remain episomal or those which integrate into the host cell genome.

Exemplary, eukaryotic expression vectors, include the cloned bovine papilloma virus genome, the cloned genomes of the murine retroviruses, and eukaryotic cassettes, such as the pSV-2 gpt system (described by Mulligan and Berg, 1979, Nature Vol. 277:108-114) the Okayama-Berg cloning system (Mol Cell Biol. Vol. 2:161-170, 1982), pGAL4, pCI (e.g., pCI-neo), and the expression cloning vector described by Genetics Institute (Science Vol. 228:810-815, 1985), are available which provide substantial assurance of at least some expression of the protein of interest in the transformed mammalian cell.

Particularly preferred are vectors which contain regulatory elements that can be linked to the inventive PTTG-encoding DNAs or PTTG-C-encoding DNA segment, for transfection of mammalian cells. Examples are cytomegalovirus (CMV) promoter-based vectors such as pcDNA1 (Invitrogen, San Diego, Calif.), MMTV promoter-based vectors such as pMAMNeo (Clontech, Palo Alto, Calif.) and pMSG (Pharmacia, Piscataway, N.J.), and SV40 promoter-based vectors such as pSVβ (Clontech, Palo Alto, Calif.).

In one embodiment of the present invention, adenovirus-transferrin/polylysine-DNA (TfAdp1-DNA) vector complexes (Wagner et al., 1992, PNAS, USA, 89.6099-6103; Curiel et al., 1992, Hum. Gene Therapy, 3:147-154; Gao et al., 1993, Hum. Gene Ther., 4:14-24) are employed to transduce mammalian cells with heterologous PTTG-specific nucleic acid. Any of the plasmid expression vectors described herein may be employed in a TfAdpl-DNA complex.

In addition, vectors may contain appropriate packaging signals that enable the vector to be packaged by a number of viral virions, e.g., retroviruses, herpes viruses, adenoviruses, resulting in the formation of a “viral vector.”

“Virus”, as used herein, means any virus, or transfecting fragment thereof, which can facilitate the delivery of the polynucleotide into mammalian cells. Examples of viruses which are suitable for use herein are adenoviruses, adeno-associated viruses, retroviruses such as human immune-deficiency virus, lentiviruses, mumps virus, and transfecting fragments of any of these viruses, and other viral DNA segments that facilitate the uptake of the desired DNA segment by, and release into, the cytoplasm of germ cells and mixtures thereof A preferred viral vector is Moloney murine leukemia virus and the retrovirus vector derived from Moloney virus called vesicular-stomatitis-virus-glycoprotein (VSV-G)-Moloney murine leukemia virus. A most preferred viral vector is a pseudotyped (VSV-G) lentiviral vector derived from the HIV virus. (Naldini et al. [1996]). Also, the mumps virus is particularly suited because of its affinity for immature sperm cells including spermatogonia. All of the above viruses may require modification to render them non-pathogenic or less antigenic. Other known viral vector systems, however, are also useful within the confines of the invention.

Viral based systems provide the advantage of being able to introduce relatively high levels of the heterologous nucleic acid into a variety of cells. Suitable viral vectors for introducing inventive PTTG-specific polynucleotides into mammalian cells (e.g., vascular tissue segments) are well known in the art. These viral vectors include, for example, Herpes simplex virus vectors (e.g., Geller et al., 1988, Science, 241:1667-1669), Vaccinia virus vectors (e.g., Piccini et al., 1987, Meth. in Enzymology, 153:545-563; Cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84), Moloney murine leukemia virus vectors (Danos et al., 1980, PNAS, USA, 85:6469), adenovirus vectors (e.g., Logan et al., 1984, PNAS, USA, 81:3655-3659; Jones et al., 1979, Cell, 17:683-689; Berkner, 1988, Biotechniques, 6:616-626; Cotten et al., 1992, PNAS, USA, 89:6094-6098; Graham et al., 1991, Meth. Mol. Biol., 7:109-127), adeno-associated virus vectors, retrovirus vectors (see, e.g., U.S. Pat. Nos. 4,405,712 and 4,650,764), and the like. Especially preferred viral vectors are the adenovirus and retroviral vectors.

As used herein, “retroviral vector” refers to the well-known gene transfer plasmids that have an expression cassette encoding an heterologous gene residing between two retroviral LTRs. Retroviral vectors typically contain appropriate packaging signals that enable the retroviral vector, or RNA transcribed using the retroviral vector as a template, to be packaged into a viral virion in an appropriate packaging cell line (see, e.g., U.S. Pat. No. 4,650,764). Retroviral vectors include lentiviral vectors, such as HIV-derived vectors.

Suitable retroviral vectors for use herein are described, for example, in U.S. Pat. No. 5,252,479, and in WIPO publications WO 92/07573, WO 90/06997, WO 89/05345, WO 92/05266 and WO 92/14829, incorporated herein by reference, which provide a description of methods for efficiently introducing nucleic acids into human cells using such retroviral vectors. Other retroviral vectors include, for example, the mouse mammary tumor virus vectors (e.g., Shackleford et al., 1988, PNAS, USA, 85:9655-9659), and the like.

A most preferred embodiment employs a pseudotyped retroviral vector system, which was developed for gene therapy. (Naldini, L., et al., In vivo gene delivery and stable transduction of nondividing cells by a lentiviral vector, Science 272: 263-267 [1996]), and which is used to transduce mammalian cells. This gene delivery system employs retroviral particles generated by a three-plasmid expression system. In this system a packaging construct contains the human cytomegalovirus (hCMV) immediate early promoter, driving the expression of all viral proteins. The construct's design eliminates the cis-acting sequences crucial for viral packaging, reverse transcription and integration of these transcripts. The second plasmid encodes a heterologous envelope protein (env), namely the G glycoprotein of the vesicular stomatitis virus (VSV-G). The third plasmid, the transducing vector (pHR′), contains cis-acting sequences of human immunodeficiency virus (HIV) required for packaging, reverse transcription and integration, as well as unique restriction sites for cloning heterologous complementary DNAs (cDNAs). For example, a genetic selection marker, such as green fluorescent protein (GFP), enhanced green fluorescent protein (EGFP), blue fluorescent protein, yellow fluorescent protein, β-galactosidase, and/or a gene encoding another preselected product is cloned downstream of the hCMV promoter in the HR'vector, and is operatively linked so as to form a transcriptional unit. A VSV-G pseudotyped retroviral vector system is capable of infecting a wide variety of cells including cells from different species and of integrating into the genome. Some retroviruses, i.e., lentiviruses, such as HIV, have the ability to infect non-dividing cells. Lentiviruses have a limited capacity for heterologous DNA sequences, the size limit for this vector being 7-7.5 kilobases (Verma, I. M. and Somia, N., Gene Therapy—promises, problems and prospects, Nature 389:239-242 [1997]). In vivo experiments with lentiviruses show that expression does not shut off like other retroviral vectors and that in vivo expression in brain, muscle, liver or pancreatic-islet cells, is sustained at least for over six months—the longest time tested so far (Verma and Somia [1997]; Anderson, W F., Human Gene Therapy, Nature (Suppl). 392:25-30 [1998]).

“Gene delivery (or transfection) mixture”, in the context of this patent, means a selected polynucleotide, whether in sense or anti-sense orientation, together with an appropriate vector mixed, for example, with an effective amount of uptake-enhancing agent as described above. (E.g., Clark et al., Polycations and cationic lipids enhance adenovirus transduction and transgene expression in tumor cells, Cancer Gene Ther. 6(5):437-46 [1999]). For example, the efficiency of adenoviral-, retroviral-, or lentiviral-mediated transduction is enhanced significantly by including a cationic lipid, such as polybrene during the infection.

In accordance with peptide-based embodiments of the inventive method of inhibiting neoplastic cellular proliferation and/or transformation or with the method of modulating angiogenesis in a tissue directed to inhibiting angiogenesis, an inventive composition is delivered comprising a PTTG carboxy-terminal peptide, which is interchangeably designated herein “PTTG-C” or “PTTG C-terminal peptide”. In accordance with the method of modulating angiogenesis in a tissue directed to enhancing angiogenesis, an inventive composition is delivered comprising a PTTG peptide instead.

The terms “protein”, “peptide”, and “polypeptide” are used interchangeably herein. As used herein, the phrase “PTTG” refers to protein member of a mammalian family of PTTG proteins, formerly also known as “pituitary-tumor-specific-gene” (PTSG) proteins, that are able to transform mammalian cells in tissue culture (e.g., NIH 3T3 and the like).

In vivo, PTTG proteins are further characterized by having the ability to induce tumor formation, for example, in nude mice (e.g., when transfected into NIH 3T3 and the like). PTTG proteins include naturally occurring allelic variants thereof encoded by mRNA generated by alternative splicing of a primary transcript, and further include fragments thereof which retain at least one native biological activity.

The term “biologically active” or “functional”, when used herein as a modifier of inventive PTTG protein(s), peptide(s), or fragments thereof, refers to a polypeptide that exhibits at least one of the functional characteristics attributed to PTTG. For example, one biological activity of PTTG is the ability to transform cells in vitro (e.g., NIH 3T3 and the like). Another biological activity is the ability to modulate the activation of mammalian T-lymphocytes, as described herein. Another biological activity of PTTG peptide is the ability to induce angiogenesis through the induction of basic fibroblast growth factor (bFGF) production and/or secretion. Production of bFGF encompasses intracellular expression or biosynthesis of bFGF, regardless of whether the bFGF is released from the bFGF-producing cell. Secretion of bFGF encompasses release of bFGF from bFGF-producing cells by active (i.e., energy-consuming) or passive (non-energy-consuming) means.

Yet another biological activity of PTTG is the ability to induce neoplastic cellular proliferation (e.g., tumorigenesis) in nude mice (e.g., when transfected into NIH-3T3 cells and the like).

On the other hand, the inventive PTTG-C peptide, as distinct from the full length native PTTG protein, has the biological activity of inhibiting PTTG-mediated tumorigenesis in a dominant negative manner “Dominant negative” is commonly used to describe a gene or protein which has a dominant effect similar to that described genetically, i.e. one copy of the gene gives a mutant phenotypic effect, and a negative effect in that it prevents or has a negative impact on a biological process such as a signal transduction pathway. Thus, PTTG carboxy-terminal peptides have the ability to downregulate intracellular PTTG expression and/or endogenous PTTG function. The inventive method is not limited to any particular biochemical, genetic, and/or physiological mechanism(s) by which a PTTG-C peptide exerts its biological activity on PTTG expression and/or PTTG function, and any or all such mechanism(s) can contribute to the biological activity of PTTG-C, in accordance with the invention.

Another biological activity of PTTG or PTTG-C peptides is the ability to act as an immunogen for the production of polyclonal and monoclonal antibodies that bind specifically to PTTG and/or PTTG-C. Thus, an inventive nucleic acid encoding PTTG or PTTG-C will encode a polypeptide specifically recognized by an antibody that also specifically recognizes a PTTG protein as described herein. Such activity may be assayed by any method known to those of skill in the art. For example, a test-polypeptide encoded by a PTTG cDNA can be used to produce antibodies, which are then assayed for their ability to bind to the protein. If the antibody binds to the test-polypeptide and the protein with substantially the same affinity, then the polypeptide possesses the requisite biological activity with respect to immunogenicity.

In the inventive method of inhibiting neoplastic cellular proliferation and/or transformation of a mammalian breast or ovarian cell, whether in vitro or in vivo, useful PTTG-C peptides encompass also any fragment of a larger PTTG-C molecule, which fragment retains PTTG-C biological activity with respect to downregulating endogenous PTTG expression and/or endogenous PTTG function. Useful PTTG-C peptides are preferably, but not exclusively, about 15 to about 60 contiguous amino acid residues long and comprise one or more proline-rich regions, which are peptide segments having a PXXP motif, where the Xs between the proline (P) residues represent any amino acid residue, including proline. The proline-rich region(s) of the PTTG-C peptide is a potential SH3-binding site.

SH3 binding sites of PTTG protein molecules are useful targets for inhibiting intracellular PTTG expression and/or PTTG protein function. Downregulation or inhibition of intracellular PTTG expression and/or endogenous PTTG function can be accomplished by blocking specific binding to a SH3-binding site normally present on endogenous PTTG protein molecules, thus interfering with SH3-mediated signal transduction in the cell.

Most preferably, the PTTG-C peptide is derived from a human PTTG, also designated hPTTG or PTTG1 protein. The native human PTTGI protein is 202 amino acids long, having the following amino acid sequence (Table 7 below; encoded by nucleotide positions 95 through 700 of human PTTG1 sequence SEQ. ID. NO.:3 and degenerate sequences).

TABLE 7
PTTG1 amino acid sequence.

| | | |
|---|---|---|
| 1 | MATLIYVDKE NGEPGTRVVA KDGLKLGSGP SIKALDGRSQ VSTPRFGKTF | (SEQ. ID. NO.:4) |
| 51 | DAPPALPKAT RKALGTVNRA TEKSVKTKGP LKQKQPSFSA KKMTEKTVKA | |
| 101 | KSSVPASDDA YPEIEKFFPF NPLDFESFDL PEEHQIAHLP LSGVPLMILD | |
| 151 | EERELEKLFQ LGPPSPVKMP SPPWESNLLQ SPSSILSTLD VELPPVCCDI | |
| 201 | DI | |

The human PTTG1 peptide is also encoded by any degenerate coding sequence encoding the amino acid sequence of SEQ. ID. NO.:4.

A preferred PTTG-C has the amino acid sequence corresponding to amino acid residues 147 through 202 of SEQ. ID. NO.:4 (Table 8 below; encoded by nucleotide positions 533 through 700 of SEQ. ID. NO.:3 or 1-168 of SEQ. ID. NO.:10 and degenerate sequences).

TABLE 8
Human PTTG-C amino acid sequence.

MILDEERELE KLFQLGPPSP VKMPSPPWES NLLQSPSSIL STLDVELPPV CCDIDI (SEQ. ID. NO.: 9).

There are at least two proline-rich regions between amino acid residues 163-173 of SEQ. ID. NO.:4, which correspond to amino acid residues 17 through 27 of SEQ. ID. NO.:9, encoded by nucleotides 49 through 81 of SEQ. ID. NO.:10 and degenerate sequences. Proline-rich regions are found at amino acid residues 163-167 and 170-173 of SEQ. ID. NO.:4, corresponding to amino acid residues 17-20 and 24-27 of SEQ. ID. NO.:9. Other useful smaller peptide fragments of SEQ. ID. NO.:9 are tested by routine means for their effectiveness in inhibiting neoplastic cellular proliferation and/or transformation of a cell.

Another example of a PTTG protein is a rat PTTG having the following amino acid sequence (Table 9 below, encoded by nucleotide positions 293-889 of SEQ. ID. NO:1 and degenerate sequences).

TABLE 9
Rat PTTG amino acid sequence.

| | |
|---|---|
| Met Ala Thr Leu Ile Phe Val Asp Lys Asp Asn Glu Glu Pro Gly Ser | 16 |
| Arg Leu Ala Ser Lys Asp Gly Leu Lys Leu Gly Ser Gly Val Lys Ala | 32 |
| Leu Asp Gly Lys Leu Gln Val Ser Thr Pro Arg Val Gly Lys Val Phe | 48 |
| Gly Ala Pro Gly Leu Pro Lys Ala Ser Arg Lys Ala Leu Gly Thr Val | 64 |
| Asn Arg Val Thr Glu Lys Pro Val Lys Ser Ser Lys Pro Leu Gln Ser | 80 |
| Lys Gln Pro Thr Leu Ser Val Lys Lys Ile Thr Glu Lys Ser Thr Lys | 96 |
| Thr Gln Gly Ser Ala Pro Ala Pro Asp Asp Ala Tyr Pro Glu Ile Glu | 112 |
| Lys Phe Phe Pro Phe Asp Pro Leu Asp Phe Glu Ser Phe Asp Leu Pro | 128 |
| Glu Glu His Gln Ile Ser Leu Leu Pro Leu Asn Gly Val Pro Leu Met | 144 |
| Ile Leu Asn Glu Glu Arg Gly Leu Glu Lys Leu Leu His Leu Asp Pro | 160 |
| Pro Ser Pro Leu Gln Lys Pro Phe Leu Pro Trp Glu Ser Asp Pro Leu | 176 |
| Pro Ser Pro Pro Ser Ala Leu Ser Ala Leu Asp Val Glu Leu Pro Pro | 192 |
| Val Cys Tyr Asp Ala Asp Ile (SEQ. ID. NO.:2). | 199 |

A rat PTTG-C peptide includes amino acid residues 144 through 199 of SEQ. ID. NO.:2, i.e., SEQ. ID. NO.:16 (Table 10 below; encoded by nucleotide positions 722 through 889 of SEQ. ID. NO.:1 or 1-168 of SEQ. ID. NO.:18 and degenerate sequences).

TABLE 10
Rat PTTG-C amino sequence.

| | | |
|---|---|---|
| Met Ile Leu Asn Glu Glu Arg Gly Leu Glu Lys Leu Leu His Leu Asp | 16 | SEQ. ID. NO.:16 |
| Pro Pro Ser Pro Leu Gln Lys Pro Phe Leu Pro Trp Glu Ser Asp Pro | 32 | |
| Leu Pro Ser Pro Pro Ser Ala Leu Ser Ala Leu Asp Val Glu Leu Pro | 48 | |
| Pro Val Cys Tyr Asp Ala Asp Ile | 56 | |

The amino acid sequence of SEQ. ID. NO.:16 includes proline-rich regions at amino acid residues 17-20, 24-27, and 34-37 (corresponding to amino acid residues 160-163, 167-170, and 177-180 of SEQ. ID. NO.:2).

Another example of a PTTG protein is a murine PTTG having the following amino acid sequence (Table 11 below; encoded by nucleotide positions 304 through 891 of SEQ ID. NO.:15 and degenerate sequences).

TABLE 11
Murine PTTG amino acid sequence.

| | |
|---|---|
| 1 | MATLIFVDKD NEEPGRRLAS KDGLKLGTGV KALDGKLQVS TPRVGKVFNA SEQ. ID. NO.:14 |
| 51 | PAVPKASRKA LGTVNRVAEK PMKTGKPLQP KQPTLTGKKI TEKSTKTQSS |
| 101 | VPAPDDAYPE IEKFFPFNPL DFDLPEEHQI SLLPLNGVPL ITLNEERGLE |
| 151 | KLLHLGPPSP LKTPFLSWES DPLYSPPSAL STLDVELPPV CYDADI |

A murine PTTG-C peptide includes amino acid residues 141 through 196 of SEQ. ID. NO.:14, i.e., SEQ. ID. NO.:17 (Table 12 below; encoded by nucleotide positions 724 through 891 of SEQ. ID. NO.:15 or 1-168 of SEQ. ID. NO.:19 and degenerate sequences).

TABLE 12.
Murine PTTG-C amino acid sequence.

| | | |
|---|---|---|
| ITLNEERGLE KLLHLGPPSP LKTPFLSWES DPLYSPPSAL STLDVELPPV CYDADI | 56 | (SEQ. ID. NO.:17). |

The amino acid sequence of SEQ. ID. NO.:17 includes a proline-rich region at amino acid residues 17-20 (corresponding to amino acid residues 157-160 of SEQ. ID NO.:14).

Preferred PTTG-C peptides include:

(A) peptides having an amino acid sequence of (SEQ. ID. NO.:9), (SEQ. ID. NO.:16), or (SEQ. ID. NO.:17); or

(B) mammalian PTTG-C peptides having at least about 60% sequence homology with any of the sequences in (A); or

(C) peptide fragments of any of the sequences in (A) or (B) that comprise at least 15 contiguous amino acid residues and that function to downregulate endogenous PTTG expression and/or PTTG function. Most preferably, the fragment of (C) includes one or more proline-rich regions.

Those of skill in the art will recognize that in other useful PTTG-C peptides numerous residues of any of the above-described PTTG or PTTG-C amino acid sequences can be substituted with other, chemically, sterically and/or electronically similar residues without substantially altering PTTG or PTTG-C biological activity In addition, larger polypeptide sequences containing substantially the same coding sequences as in SEQ ID NO:2, SEQ. ID. NO.:4, SEQ. ID. NO.:9, SEQ. ID. NO.:14, SEQ. ID. NO.:16, or SEQ. ID. NO.:17 (e.g., splice variants) are contemplated.

As employed herein, the term “substantially the same amino acid sequence” refers to amino acid sequences having at least about 60% sequence homology or identity with respect to any of the amino acid sequences described herein (“reference sequences”), and retaining comparable functional and biological activity characteristic of the protein defined by the reference sequences described, particularly with respect to neoplastic cellular proliferation and/or transformation or its inhibition. More preferably, proteins having “substantially the same amino acid sequence” will have at least about 80%, still more preferably about 90% amino acid identity with respect to a reference amino acid sequence; with greater than about 95% amino acid sequence identity being especially preferred. It is recognized, however, that polypeptide containing less than the described levels of sequence identity arising as splice variants or that are modified by conservative amino acid substitutions are also encompassed within the scope of the present invention. The degree of sequence homology is determined by conducting an amino acid sequence similarity search of a protein data base, such as the database of the National Center for Biotechnology Information (NCBI; www.ncbi.nlm.nih.gov/BLAST/), using a computerized algorithm, such as PowerBLAST, QBLAST, PSI-BLAST, PHI-BLAST, gapped or ungapped BLAST, or the “Align” program through the Baylor College of Medicine server (www.hgsc.bcm.tmc.edu/seq_data). (E.g., Aitchul, S. F., et al., Gapped BLAST and PSI-BLAST: a new generation of protein database search programs, Nucleic Acids Res. 25(17):3389-402 [1997]; Zhang, J., & Madden, T. L., PowerBLAST: a new network BLAST application for interactive or automated sequence analysis and annotation, Genome Res. 7(6):649-56 [1997]; Madden, T. L., et al., Applications of network BLAST server, Methods Enzymol. 266:131-41 [1996]; Altschul, S. F., et al, Basic local alignment search tool, J. Mol. Biol. 215(3):403-10 [1990]).

Also encompassed by the terms PTTG protein or PTTG-C peptide, respectively, are biologically functional or active peptide analogs thereof The term peptide “analog” includes any polypeptide having an amino acid residue sequence substantially identical to a sequence specifically shown herein in which one or more residues have been conservatively substituted with a functionally similar residue and which displays the ability to mimic the biological activity of PTTG or PTTG-C, respectively, particularly with respect to neoplastic cellular proliferation and/or transformation or its inhibition, or with respect to immunosuppressing mammalian lymphocyte cells, as described herein above. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue, provided that such polypeptide displays the requisite biological activity.

“Chemical derivative” refers to a subject polypeptide having one or more residues chemically derivatized by reaction of a functional side group. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine. The inventive polypeptide of the present invention also include any polypeptide having one or more additions and/or deletions of residues, relative to the sequence of a polypeptide whose sequence is shown herein, so long as the requisite PTTG or PTTG-C biological activity is maintained.

In accordance with peptide-based embodiments of the inventive method of inhibiting neoplastic cellular proliferation and/or transformation, the composition comprising the PTTG-C peptide is delivered to the cell. A suitable amount of the inventive PTTG-C peptide to be delivered to the cells, in accordance with the method, preferably ranges from about 0.1 nanograms to about 1 milligram per gram of tumor tissue, in vivo, or about 0.1 nanograms to about 1 microgram per 5000 cells, in vitro. Suitable amounts for particular varieties of PTTG-C peptide and/or cell types and/or for various individual mammalian subjects undergoing treatment, can be determined by routine experimentation.

Methods of delivering and importing peptides into target cells are known. For example, the composition preferably, but not necessarily, comprises in addition to the PTTg or PTTG-C peptide, a complex in which the PTTG or PTTG-C peptide is complexed with a cellular uptake-enhancing agent. For example, the PTTG or PTTG-C peptide can be covalently linked in a complex to a cellular uptake-enhancing and/or importation-competent peptide segment for delivery of PTTG or PTTG-C into the mammalian cell; in addition, a nuclear localization peptide can be included in the complex to direct the PTTG or PTTG-C to the nucleus. (E.g., Lin et al., Method for importing biologically active molecules into cells, U.S. Pat. No. 6,043,339). An “importation-competent peptide,” as used herein, is a sequence of amino acids generally of a length of about 10 to about 50 or more amino acid residues, many (typically about 55-60%) residues of which are hydrophobic such that they have a hydrophobic, lipid-soluble portion. The hydrophobic portion is a common, major motif of a signal peptide, and it is often recognizable as a central part of the signal peptide of a protein secreted from cells. A signal peptide is a peptide capable of penetrating through the cell membrane to allow the export of cellular proteins. Signal peptides useful in the present method are also “importation-competent,” i.e., capable of penetrating through the cell membrane from outside the cell to the interior of the cell.

In a preferred embodiment, a PTTG or PTTG-C peptide forms first PTTG or PTTG-C peptide segment of a chimeric or fusion protein. The chimeric or fusion protein comprises at least the first PTTG or PTTG-C peptide segment and a second cellular uptake-enhancing and/or importation-competent peptide segment. The second segment of the chimeric or fusion protein is a cellular uptake-enhancing and/or importation-competent peptide segment, such as a signal peptide, that allows the hybrid molecule to enter cells, whether in vitro or in vivo. The second peptide segment, such as the human immunodeficiency virus (HIV) TAT protein (Schwarze, S. R., et al., In vivo protein transduction: delivery of a biologically active protein into the mouse, Science 285:1569-72 [1999]), infiltrates the cells, and once within the cells, the PTTG-C peptide segment of the fusion protein becomes active within the cells to inhibit endogenous PTTG expression and/or PTTG function. Another example of a useful uptake-enhancing peptide segment is the signal peptide from Kaposi fibroblast growth factor (K-FGF). But any cellular uptake-enhancing and/or importation-competent peptide segment, capable of translocating across the cell membrane into the interior of the selected target mammalian cell, can be used according to this invention. The chimeric or fusion protein can also include additional segments, such as a linker segment, that can be an intervening segment between the first and second segments. The additional segment can alternatively be a terminal segment, as appropriate.

In embodiments of the method involving the use of PTTG or PTTG-C chimeric or fusion proteins, the cellular uptake-enhancing and/or importation-competent peptide segment can be the uptake-enhancing agent. Alternatively, or in addition, the cellular uptake-enhancing agent can be a lipid or liposome uptake-enhancing agent as described herein above, such as lipofectin, lipofectamine, DOTAP, and others. Cationic (or polycationic) lipids or liposomes can also be complexed with a signal peptide and a negatively-charged biologically active molecule by mixing these components and allowing them to charge-associate. Anionic liposomes generally are utilized to encapsulate within the liposome the substances to be delivered to the cell. Procedures for forming cationic liposome- encapsulating substances are standard in the art and can readily be utilized herein by one of ordinary skill in the art to encapsulate the complex of this invention. For example, liposome uptake-enhancing agents complexed with inventive PTTG or PTTG-C peptide fragments can be encapsulated in polyethylene glycol (PEG), FuGENE6, or the like.

With respect to delivery of the inventive composition (whether containing a PTTG-encoding polynucleotide, PTTG-C-related polynucleotide, PTTG peptide, or PTTG-C peptide or active fragment) to mammalian cells in vivo, the composition is administered to a mammalian subject in need of treatment, including a human subject, by any conventional delivery route. Preferably, the PTTG-encoding polynucleotide, PTTG-C-related polynucleotide, PTTG peptide, or PTTG-C peptide, whether or not complexed with cellular uptake-enhancing and/or importation-competent peptides (e.g., signal or localization peptides), is injected intravenously, intra-arterially, intraperitoneally, or by means of injection directly into a tumor or into a cell by microinjection. For example, direct injection of the inventive composition into the tumor is preferred for breast or ovarian tumors in vivo. Conventional stereotactic methods can be useful for direct injection into tumors or cells. Administration by nasal, rectal, or vaginal delivery routes can also be useful. Administration by catheter or stent can also be useful for delivering the composition containing the PTTG-encoding DNA, PTTG-C-related polynucleotide, PTTG peptide, or PTTG-C peptide.

In other preferred embodiments, controlled release formulations of biodegradable polymeric microspheres or nanospheres (e.g., polylactide-co-glycolide; PLGA) encapsulating the PTTG peptide, PTTG-C peptide, PTTG fusion protein, or PTTG-C chimeric or fusion protein are administered to the mammalian subject orally. (E.g., Zhu, G. et al., Stabilization of proteins encapsulated in injectable poly(lactide-co-glycolide), Nature Biotechnology 18:52-57 [2000]).

In some embodiments, isolated and crystallized PTTG peptide or PTTG-C peptide can be cross-linked with a multifunctional crosslinking agent that inhibits proteolysis of the peptide in vivo. (Navia, M. A., Method of protein therapy by orally administering crosslinked protein crystals, U.S. Pat. No. 6,011,001).

Some useful embodiments of the method of inhibiting neoplastic cellular proliferation and/or transformation of mammalian breast or ovarian cells include further administering a cytotoxic chemotherapeutic agent to the cell simultaneously with or after delivering to the cell the PTTG carboxy-terminal-related polynucleotide (which in some embodiments is comprised in an expression vector), or the PTTG carboxy terminal (PTTG-C) peptide or the biologically functional fragment thereof Because the presence of intracellular PTTG-C peptide hypersensitizes the cell to the cytotoxic chemotherapeutic agent, the practitioner can decrease the typical effective dose of the cytotoxic chemotherapeutic agent by about 10- to 100-fold, compared to the conventional dose, thereby minimizing damage to non-malignant tissue from cytotoxic chemotherapeutic agents.

Consequently, in other embodiments of the method that further comprise delivering the inventive composition containing the PTTG carboxy-terminal-related polynucleotide or the PTTG carboxy terminal (PTTG-C) peptide (or the biologically functional fragment thereof) to the cell in vivo within a subject, systemic toxic effects from anti-cancer medication are lessened in a particular subject receiving treatment with the lower effective dose of cytotoxic chemotherapeutic agent. Therefore, the quality of life and the likelihood of survival for the subject can be improved.

The cytotoxic chemotherapeutic agent includes but is not limited to paclitaxel (Taxol), 5-fluorouracil, cisplatin, carboplatin, methotrexate, daunorubicin, doxorubicin, vincristine, vinblastine, or a cytotoxic alkylating agent, such as, but not limited to, busulfan (1,4-butanediol dimethanesulphonate; Myleran, Glaxo Wellcome), chlorambucil, cyclophosphamide, melphalan, or ethyl ethanesulfonic acid. The cytotoxic chemotherapeutic agent is administered to the cell by conventional means, but in an effective dose about 10- to 100-fold lower than the conventional dose for a given cytotoxic chemotherapeutic agent. Administration of the cytotoxic chemotherapeutic agent can be simultaneous with or after delivery of the inventive composition as long as biologically active PTTG-C peptide is still present in the cell.

In accordance with the inventive method of inhibiting neoplastic cellular proliferation and/or transformation that is mediated by PTTG, the mammalian cell is a cell that overexpresses PTTG, the gene that encodes a PTTG protein. Although detecting PTTG overexpression by the cell is not essential or necessary to the practice of the inventive method, the level of PTTG expression, including overexpression, is detectable by one skilled in the art.

Detection of PTTG expression is accomplished by immunochemical assay for PTTG protein, for example, using the inventive anti-PTTG-C antibodies, described herein, or other anti-PTTG-specific antibodies. Alternatively, amplification of PTTG-specific mRNAs present in biological samples (e.g., tissue biopsy) can be used to detect PTTG expression. This is done by known molecular biological techniques of amplification and analysis of the amplification products for the presence or absence of PTTG-specific amplification products. If PTTG gene-specific amplification products are present, the findings are indicative of expression of the PTTG gene and diagnostic of the presence of neoplastic cellular proliferation in the subject as defined herein.

However, for interpretation of negatives (no PTTG-specific amplification products) analysis is preferably carried out following a control amplification of nucleic acids specific for a housekeeping gene, for example, a gene encoding β-actin, phosphofructokinase (PFK), glyceraldehyde 3-phosphate dehydrogenase, or phosphoglycerate kinase. Only if expression of the housekeeping gene is detected in the sample, is the absence of PTTG gene expression reliably accepted. With increasing sensitivity of amplification and analysis methods employed, it becomes increasingly preferable to determine the level of PTTG gene expression relative to expression of a housekeeping gene, in order to better distinguish neoplastic, hyperplastic, cytologically dysplastic and/or premalignant cellular growth or proliferation from the detectable background of normal cellular division. The ratio of PTTG expression to housekeeping gene expression is determined, for example, by real-time PCR methods or densitometric measurement and analysis of electrophoretic bands after amplification. When the ratio of PTTG expression to housekeeping gene expression exceeds a normal cell standard range and/or approximates an abnormal (e.g., neoplastic) cell standard range, this indicates overexpression of PTTG gene product, characteristic of neoplastic, hyperplastic, cytologically dysplastic and/or premalignant cellular growth or proliferation.

PTTG-specific mRNAs in a biological sample are amplified by a suitable amplification method. For example, a reverse transcriptase-mediated polymerase chain reaction (RT-PCR) is employed to amplify PTTG-specific nucleic acids. Briefly, two enzymes are used in the amplification process, a reverse transcriptase to transcribe PTTG-specific cDNA from a PTTG-specific mRNA template in the sample, a thermal resistant DNA polymerase (e.g., Taq polymerase), and PTTG-specific primers to amplify the cDNA to produce PTTG gene-specific amplification products. The use of limited cycle PCR yields semi-quantitative results. (E.g., Gelfand et al., Reverse transcription with thermostable DNA polymerase-high temperature reverse transcription, U.S. Pat. Nos. 5,310,652; 5,322,770; Gelfand et al., Unconventional nucleotide substitution in temperature selective RT-PCR, U.S. Pat. No. 5,618,703).

Alternatively, single enzyme RT-PCR is employed to amplify PTTG gene-specific nucleic acids. Single enzymes now exist to perform both reverse transcription and polymerase functions, in a single reaction. For example, the Perkin Elmer recombinant Thermus thermophilus (rTth) enzyme(Roche Molecular), or other similar enzymes, are commercially available.

Real-time RT-PCR can be employed to amplify PTTG-specific nucleic acids. Briefly, this is a quantitative gene analysis based on the ratio of PTTG gene expression and the expression of a housekeeping gene, i.e., a gene that is expressed at about the same level in normal and abnormal (e.g., malignant) cells, for example, a gene encoding β-actin, phosphofructokinase, glyceraldehyde 3-phosphate dehydrogenase, or phosphoglyceratekinase. The the ratio of the PTTG and housekeeping genes' expressions is routinely established as a standard for normal and abnormal cells, which standard expression ratio(s) is (are) used for comparison in determining that expression of the PTTG gene relative to expression of the “housekeeping” gene in a given sample is either “normal” or “increased”, the latter indicative of “overexpression” and diagnostic for the presence of neoplastic, hyperplastic, cytologically dysplastic and/or premalignant cellular growth or proliferation. In this embodiment, the ratio is the key to diagnosis and constitutes quantitative gene expression analysis. This embodiment utilizes so-called real-time quantitative PCR, carried out with commercially available instruments, such as the Perkin Elmer ABI Prism 7700, the so-called Light Cycler (Roche Molecular), and/or other similar instruments. Optionally, single enzyme RT-PCR technology, for example, employing rTth enzyme, can be used in a real-time PCR system. Preferably, amplification and analysis are carried out in an automated fashion, with automated extraction of mRNA from a urine sediment sample, followed by real-time PCR, and fluorescence detection of amplification products using probes, such as TaqMan or Molecular Beacon probes. Typically, the instrumentation includes software that provides quantitative analytical results during or directly following PCR without further amplification or analytical steps.

Alternatively, transcription-mediated amplification (TMA) is employed to amplify PTTG gene-specific nucleic acids. (E.g., K. Kamisango et al., Quantitative detection of hepatitis B virus by transcription-mediated amplification and hybridization protection assay, J. Clin. Microbiol. 37(2):310-14 [1999]; M. Hirose et al., New method to measure telomerase activity by transcription-mediated amplification and hybridization protection assay, Clin. Chem. 44(12)2446-52 [1998]). Rather than employing RT-PCR for the amplification of a cDNA, TMA uses a probe that recognizes a PTTG-specific (target sequence) RNA; in subsequent steps, from a promoter sequence built into the probe, an RNA polymerase repetitively transcribes a cDNA intermediate, in effect amplifying the original RNA transcripts and any new copies created, for a level of sensitivity approaching that of RT-PCR. The reaction takes place isothermally (one temperature), rather than cycling through different temperatures as in PCR.

Other useful amplification methods include a reverse transcriptase-mediated ligase chain reaction (RT-LCR), which has utility similar to RT-PCR. RT-LCR relies on reverse transcriptase to generate cDNA from mRNA, then DNA ligase to join adjacent synthetic oligonucleotides after they have bound the target cDNA.

Amplification of a PTTG gene-specific nucleic acid segment of the subject can be achieved using PTTG gene-specific oligonucleotide primers and primer sets as provided herein.

Optionally, high throughput analysis may be achieved by PCR multiplexing techniques well known in the art, employing multiple primer sets, for example primers directed not only to PTTG gene-specific nucleic acids, but to amplifying expression products of housekeeping genes (controls) or of other potential diagnostic markers (e.g., oncogenes), as well, such as MAG or telomerase, to yield additional diagnostic information. (E.g., Z. Lin et al., Multiplex genotype determination at a large number of gene loci, Proc. Natl. Acad. Sci. USA 93(6):2582-87 [1996]; Demetriou et al., Method and probe for detection of gene associated with liver neoplastic disease, U.S. Pat. No. 5,866,329).

Hybridization analysis is a preferred method of analyzing the amplification products, employing one or more PTTG-specific probe(s) that, under suitable conditions of stringency, hybridize(s) with single stranded PTTG-specific nucleic acid amplification products comprising complementary nucleotide sequences. Hybridization refers to the binding of complementary strands of nucleic acid (i.e., sense:antisense strands or probe:target-DNA) to each other through hydrogen bonds, similar to the bonds that naturally occur in chromosomal DNA. The amplification products are typically deposited on a substrate, such as a cellulose or nitrocellulose membrane, and then hybridized with labeled PTTG-specific probe(s), optionally after an electrophoresis. Conventional dot blot, Southern, Northern, or fluorescence in situ (FISH) hybridization protocols, in liquid hybridization, hybridization protection assays, or other semi-quantitative or quantitative hybridization analysis methods are usefully employed along with the PTTG gene-specific probes of the present invention. Preferred probe-based hybridization conditions comprise a temperature of about 37° C., a formamide concentration of about 20%, and a salt concentration of about 5× standard saline citrate (SSC; 20×SSC contains 3 M sodium chloride, 0.3 M sodium citrate, pH 7.0). Such conditions will allow the identification of sequences which have a substantial degree of similarity with the probe sequence, without requiring perfect homology. The phrase “substantial similarity” refers to sequences which share at least 50% homology. Stringency levels used to hybridize a given probe with target-DNA can be readily varied by those of skill in the art. Preferably, hybridization conditions will be selected which allow the identification of sequences having at least about 60% homology with the probe, while discriminating against sequences which have a lower degree of homology with the probe.

As used herein, a “probe” is single-stranded DNA or RNA, or a nucleic acid analog. The inventive probe is preferably 7 to 500 nucleotides long, more preferably 14 to 150 nucleotides long, and most preferably at least 50 nucleotides long. The probe comprises, for at least part of its length, a PTTG-specific nucleotide sequence at least 7 to 15 contiguous nucleotides long, such that the probe hybridizes to a PTTG-specific single stranded nucleic acid under suitably stringent hybridization conditions. Examples of PTTG-specific nucleotide sequences are set forth in any of SEQ. ID. NOS.: 1, 3, 10, 15, 18, or 19, preferably, but not necessarily, including 5′ and/or 3′ coding regions thereof In addition, the entire cDNA encoding region of an inventive PTTG-specific nucleotide sequence, or the entire sequence corresponding to SEQ. ID. NOS.:1, 3, 10, 15, 18, 19, or nucleotide sequences complementary to any of these, can be used as a probe. For example, probes comprising inventive oligonucleotide primer sequences, such as, but not limited to, SEQ. ID. NO.:8, can be labeled for use as probes for detecting or analyzing PTTG-specific nucleic acid amplification products. Any of the inventive isolated PTTG-C-related polynucleotides can be used as probes or primers.

Alternatively, electrophoresis for analyzing amplification products is done rapidly and with high sensitivity by using any of various methods of conventional slab or capillary electrophoresis, with which the practitioner can optionally choose to employ any facilitating means of nucleic acid fragment detection, including, but not limited to, radionuclides, UV-absorbance or laser-induced fluorescence. (K. Keparnik et al, Fast detection of a (CA)18 microsatellite repeat in the IgE receptor gene by capillary electrophoresis with laser-induced fluorescence detection, Electrophoresis 19(2);249-55 [1998]; H. Inoue et al, Enhanced separation of DNA sequencing products by capillary electrophoresis using a stepwise gradient of electric field strength, J. Chromatogr. A. 802(1):179-84 [1998]; N. J. Dovichi, DNA sequencing by capillary electrophoresis, Electrophoresis 18(12-13):2393-99 [1997]; H. Arakawa et al., Analysis of single-strand conformation polymorphisms by capillary electrophoresis with laser induced fluorescence detection, J. Pharm. Biomed. Anal. 15(9-10): 1537-44 [1997]; Y. Baba, Analysis of disease-causing genes and DNA-based drugs by capillary electrophoresis. Towards DNA diagnosis and gene therapy for human diseases, J. Chromatgr B. Biomed. Appl. 687(2):271-302 [1996]; K. C. Chan et al., High-speed electrophoretic separation of DNA fragments using a short capillary, J. Chromatogr B. Biomed. Sci. Appl. 695(1): 13-15 [1997]). Probes can be labeled by methods well-known in the art.

As used herein, the terms “label”, “tracer”, and “indicating means” in their various grammatical forms refer to single atoms and molecules that are either directly or indirectly involved in the production of a detectable signal. Any label or indicating means can be linked to PTTG-specific probes, primers, or amplification products, or PTTG proteins, peptides, peptide fragments, or anti-PTTG antibody molecules. The label can be used alone or in conjunction with additional reagents. Such labels are themselves well-known in the art. The label can be a fluorescent labeling agent that chemically binds to antibodies or antigens without denaturation to form a fluorochrome (dye) that is a useful immunofluorescent tracer. A description of immunofluorescent analytic techniques is found in DeLuca, “Immunofluorescence Analysis”, in Antibody As a Tool, Marchalonis et al., eds., John Wiley & Sons, Ltd., pp. 189-231 (1982), which is incorporated herein by reference. Any of diverse fluorescent dyes can optionally be used as a label, including but not limited to, SYBR Green I, Y1O-PRO-1, thiazole orange, Hex (i.e., 6-carboxy-2′,4′,7′,4,7-hexachlorofluoroscein), pico green, edans, fluorescein, FAM (i.e., 6-carboxyfluorescein), or TET (i.e., 4,7,2′,7′-tetrachloro-6-carboxyfluoroscein). (E.g., J. Skeidsvoll and P. M. Ueland, Analysis of double-stranded DNA by capillary electrophoresis with laser-induced fluorescence detection using the monomeric dye SYBR green I, Anal. Biochem. 231(20):359-65 [1995]; H. Iwahana et al., Multiple fluorescence-based PCR-SSCP analysis using internal fluorescent labeling of PCR products, Biotechniques 21(30:510-14, 516-19 [1996]).

The label can also be an enzyme, such as horseradish peroxidase (HRP), glucose oxidase, β-galactosidase, and the like. Alternatively, radionuclides are employed as labels. The linking of a label to a substrate, i.e., labeling of nucleic acid probes, antibodies, polypeptide, and proteins, is well known in the art. For instance, an invention antibody can be labeled by metabolic incorporation of radiolabeled amino acids provided in the culture medium. See, for example, Galfre et al., Meth. Enzymol., 73:3-46 (1981). Conventional means of protein conjugation or coupling by activated functional groups are particularly applicable. See, for example, Aurameas et al., Scand. J. Immunol., Vol. 8, Suppl. 7:7-23 (1978), Rodwell et al., Biotech., 3:889-894 (1984), and U.S. Pat. No. 4,493,795.

In accordance with yet another embodiment of the present invention, there are provided anti-PTTG antibodies having specific reactivity with PTTG polypeptides of the present invention. Antibody fragments, for example Fab, Fab′, F(ab′)₂, or F(v) fragments, that selectively or specifically bind a PTTG protein, PTTG-C peptide, or immunogenic fragment of PTTG-C, are also encompassed within the definition of “antibody”.

Inventive antibodies can be produced by methods known in the art using PTTG polypeptide, proteins or portions thereof, such as PTTG-C peptide or immunogenic fragments of PTTG-C, as antigens. For example, polyclonal and monoclonal antibodies can be produced by methods well known in the art, as described, for example, in Harlow and Lane, Antibodies: A Laboratory Manual (Cold Spring Harbor Laboratory [1988]), which is incorporated herein by reference. Isolated or purified PTTG proteins, PTTG-C peptides, and immunogenic PTTG-C fragments can be used as immunogens in generating such specific antibodies.

PTTG proteins or peptides, PTTG-C peptides, or polypeptide analogs thereof, are purified or isolated by a variety of known biochemical means, including, for example, by the recombinant expression systems described herein, precipitation, gel filtration, ion-exchange, reverse-phase and affinity chromatography, and the like. Other well-known methods are described in Deutscher et al., Guide to Protein Purification: Methods in Enzymology Vol. 182, (Academic Press, [1990]), which is incorporated herein by reference. Isolated PTTG proteins or PTTG-C peptides are free of cellular components and/or contaminants normally associated with a native in vivo environment.

Isolated PTTG proteins or PTTG-C peptides can also be chemically synthesized For example, synthetic polypeptide can be produced using Applied Biosystems, Inc. Model 430A or 431A automatic peptide synthesizer (Foster City, Calif.) employing the chemistry provided by the manufacturer. Alternatively, PTTG can be isolated or purified from native sources, and PTTG-C peptides can be isolated from PTTG (or from chimeric proteins) by the use of suitable proteases.

Alternatively, PTTG or PTTG-C polypeptides can be recombinantly derived, for example, produced by mammalian cells genetically modified to express PTTG-C-encoding polynucleotides in accordance with the inventive technology as described herein. Recombinant methods are well known, as described, for example, in Sambrook et al., supra., 1989). An example of the means for preparing the inventive PTTG or PTTG-C polypeptide(s) is to express nucleic acids encoding the PTTG protein or PTTG-C peptide in a suitable host cell, such as a bacterial cell, a yeast cell, an amphibian cell (i.e., oocyte), or a mammalian cell, such as the inventive mammalian host cell described herein below, using methods well known in the art, and recovering the expressed polypeptide, again using well-known methods.

The immunogenicity of various PTTG-C fragments of interest is determined by routine screening. Alternatively, synthetic PTTG or PTTG-C polypeptides or fragments thereof can be prepared (using commercially available synthesizers) and used as immunogens. Amino acid sequences can be analyzed by methods well known in the art to determine whether they encode hydrophobic or hydrophilic domains of the corresponding polypeptide. Altered antibodies such as chimeric, humanized, CDR-grafted or bifunctional antibodies can also be produced by methods well known in the art. Such antibodies can also be produced by hybridoma, chemical synthesis or recombinant methods described, for example, in Sambrook et al., supra., and Harlow and Lane, supra. Both anti-peptide and anti-fusion protein antibodies can be used. (see, for example, Bahouth et al., Trends Pharmacol. Sci. 12:338 [1991]; Ausubel et al., Current Protocols in Molecular Biology (John Wiley and Sons, N.Y. [1989] which are incorporated herein by reference).

Antibody so produced can be used, inter alia, in diagnostic or assay methods and systems to detect the level of PTTG protein, PTTG-C peptide, or immunogenic fragments thereof, present in a mammalian, preferably human, biological sample, such as tissue or vascular fluid. This is useful, for example, in determining the level of PTTG expression. Such antibodies can also be used for the immunoaffinity or affinity chromatography purification of the inventive PTTG proteins or PTTG-C peptides. In addition, methods are contemplated herein for detecting the presence of PTTG protein or PTTG-C peptide, either on the surface of a cell or within a cell (such as within the nucleus), which methods comprise contacting the cell with an antibody that specifically binds to PTTG protein or PTTG-C peptide, under conditions permitting specific binding of the antibody to PTTG protein or PTTG-C peptide, detecting the presence of the antibody bound to PTTG or PTTG-C, and thereby detecting the presence of PTTG or PTTG-C polypeptide on the surface of, or within, the cell. With respect to the detection of such polypeptide, the antibodies can be used for in vitro diagnostic or assay methods, or in vivo imaging methods.

Immunological procedures useful for in vitro detection of target PTTG or PTTG-C polypeptides in a sample include immunoassays that employ a detectable antibody. Such immunoassays include, for example, ELISA, immunofluorescence assay (IFA), Pandex microfluorimetric assay, agglutination assays, flow cytometry, serum diagnostic assays and immunohistochemical staining procedures which are well known in the art. An antibody can be made detectable by various means well known in the art. For example, a detectable marker can be directly or indirectly attached to the antibody. Useful markers include, for example, radionuclides, enzymes, fluorogens, chromogens and chemiluminescent labels.

Inventive anti-PTTG or anti-PTTG-C antibodies are also contemplated for use herein to modulate activity of the PTTG polypeptide in living animals, in humans, or in biological tissues or fluids isolated therefrom. Accordingly, compositions comprising a carrier and an amount of an antibody having specificity for PTTG polypeptide effective to block naturally occurring ligands or other PTTG-binding proteins from binding to invention PTTG polypeptide are contemplated herein. For example, a monoclonal antibody directed to an epitope of PTTG polypeptide molecules present on the surface of a cell and having an amino acid sequence substantially the same as an amino acid sequence for a cell surface epitope of an PTTG polypeptide including the amino acid sequence shown in SEQ ID NOS:2, 4, 9, 14, 16, or 17 can be useful for this purpose.

The present invention also relates to transfected, transduced, or otherwise transformed mammalian host cells, including lymphocyte and non-lymphocyte cells such as breast or ovarian cells, comprising any of the inventive PTTG-C-related polynucleotide-containing compositions as described herein above. The inventive cells are either contained in a mammalian subject or are cultured in vitro. Included among preferred embodiments are mammalian host cells containing an expression vector comprising the inventive PTTG-C-related polynucleotide in a transcriptional unit. Preferably, a product is expressed by the cell, which product, most preferably, but not necessarily, is a biologically active PTTG-C peptide that functions to downregulate PTTG-mediated neoplastic cellular proliferation and/or transformation.

In vitro and in vivo methods of gene delivery, i.e., transfecting, transducing, or transforming suitable host cells are generally known in the art. Methods for culturing cells, in vitro, are also well known. Exemplary methods of transfection, transduction, or transformation include, e.g., infection employing viral vectors (see, e.g., U.S. Pat. Nos. 4,405,712 and 4,650,764), calcium phosphate transfection (U.S. Pat. Nos. 4,399,216 and 4,634,665), dextran sulfate transfection, electroporation, lipofection (see, e.g., U.S. Pat. Nos. 4,394,448 and 4,619,794), cytofection, microparticle bombardment or “ballistic” gene delivery (e.g., using commercially available kits, such as Accell [Powderject Vaccines, Inc.] or Helios [Bio-Rad]), and the like. The heterologous nucleic acid can optionally include sequences which allow for its extrachromosomal (i.e., episomal) maintenance, or heterologous DNA can be caused to integrate into the genome of the host (as an alternative means to ensure stable maintenance in the host cell).

The present invention further provides transgenic non-human mammals containing the inventive mammalian cells that are capable of expressing exogenous nucleic acids encoding PTTG polypeptides, particularly the inventive PTTG-C peptides and functional fragments thereof as described hereinabove. As employed herein, the phrase “exogenous nucleic acid” refers to nucleic acid sequence which is not native to the host, or which is present in the host in other than its native environment (e.g., as part of a genetically engineered DNA construct). Methods of producing transgenic non-human mammals are known in the art. Typically, the pronuclei of fertilized eggs are microinjected in vitro with foreign, i.e., xenogeneic or allogeneic DNA or hybrid DNA molecules, and the microinjected fertilized eggs are then transferred to the genital tract of a pseudopregnant female to gestate to term. (E.g., P. J. A. Krimpenfort et al., Transgenic mice depleted in mature T-cells and methods for making transgenic mice, U.S. Pat. Nos. 5,175,384 and 5,434,340; P. J. A. Krimpenfort et al., Transgenic mice depleted in mature lymphocytic cell-type, U.S. Pat. No. 5,591,669). Alternatively, methods for producing transgenic non-human mammals can involve genetic modification of female or male germ cells using an expression vector, which germ cells are then used to produce zygotes, which are gestated to term. The resulting offspring are selected for the desired phenotype. These offspring can further be bred or cloned to produce additional generations of transgenic animals with the desired phenotype. The inventive transgenic non-human mammals, preferably, but not necessarily, are large animals such as bovines, ovines, porcines, equines, and the like, that produce relatively large quantities of PTTG-C peptides that can be harvested for use in practicing the method of inhibiting neoplastic cellular proliferation and/or transformation.

Most preferably, the transgenic non-human mammal is a female that produces milk into which the inventive PTTG peptides or PTTG-C peptides have been secreted. The PTTG-C peptides are then purified from the milk. (E.g., Christa, L., et al., High expression of the human hepatocarcinoma-intestine-pancreas/pancreatic-associated protein (HIPPAP) gene in the mammary gland of lactating transgenic mice secretion into the milk and purification of the HIP/PAP lectin, Eur. J. Biochem. 267(6): 1665-71 [2000]; Sobolev, A. S. et al., Receptor-mediated transfection of murine and ovine mammary glands in vivo, J. Biol. Chem. 273(14):7928-33 [1998]; Zhang, K. et al., Construction of mammary gland-specific expression vectors for human clotting factor IX and its secretory expression in goat milk, Chin. J. Biotechnol. 13(4):271-6 [1997]; Clark, A. J., Gene expression in the mammary glands of transgenic animals, Biochem. Soc. Symp. 63:133-40 [1998]; Niemann, H. et al., Expression of human blood clotting factor VIII in the mammary gland of transgenic sheep, Transgenic Res. 8(3):237-47 [1999]).

Techniques for obtaining the preferred transgenic female mammals typically employ transfection with an expression vector in which, within a transcriptional unit regulated, for example, by a suitable β-lactoglobulin promoter, the PTTG peptide-encoding or PTTG-C peptide-encoding polynucleotide is chimerically linked with a polynucleotide encoding a mammary secretory signal peptide, such that mammary-specific expression yields a chimeric polypeptide from which the desired PTTG or PTTG-C peptide segment is removed proteolytically and purified.

The present invention is also directed to a kit for the treatment of neoplastic cellular proliferation or a kit for the modulation of angiogenesis. The kit is useful for practicing the inventive methods. The kit is an assemblage of materials or components, including at least one of the inventive compositions containing a PTTG-encoding polynucleotide, PTTG-C-related polynucleotide and/or PTTG peptides or PTTG-C peptides, as described above. The exact nature of the components configured in the inventive kit depends on its intended purpose. For example, some embodiments of the kit are configured for the purpose of treating cultured mammalian cells. Other embodiments are configured for the purpose of treating mammalian cells in vivo, i.e., for treating mammalian subjects in need of treatment, for example, subjects with malignant tumors. Preferred embodiments are directed to treating malignancies, such as breast or ovarian cancers. Other embodiments are directed to modulating angiogenesis in non-malignant tissues. In a most preferred embodiment, the kit is configured particularly for the purpose of treating human subjects.

Some embodiments of the kit include a kit for the treatment of neoplastic cellular proliferation of T-lymphocytes, which includes a composition comprising a tamed HIV vector operatively linked to a PTTG carboxy-terminal-related polynucleotide; and instructions for the use of said composition for inhibiting neoplastic cellular proliferation and/or transformation of T-lymphocytes.

Also included within the present invention is a kit for immunosuppressive therapy, which contains a composition comprising a tamed HIV vector operatively linked to a PTTG carboxy-terminal-related polynucleotide; and instructions for using the composition for inhibiting the activation of T-lymphocytes.

Instructions for use are included in the kit. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like, typically for an intended purpose.

Optionally, the kit also contains other useful components, such as, diluents, buffers, pharmaceutically acceptable carriers, specimen containers, syringes, stents, catheters, pipetting or measuring tools, paraphernalia for concentrating, sedimenting, or fractionating samples, or the inventive antibodies, and/or primers and/or probes for controls.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures.

The components are typically contained in suitable packaging material(s). As employed herein, the phrase “packaging material” refers to one or more physical structures used to house the contents of the kit, such as invention nucleic acid probes or primers, and the like. The packaging material is constructed by well known methods, preferably to provide a sterile, contaminant-free environment.

The packaging materials employed in the kit are those customarily utilized in polynucleotide-based or peptide-based systems. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing nucleic acid or peptide components. The packaging material generally has an external label which indicates the contents and/or purpose of the kit and/or its components.

The invention will now be described in greater detail by reference to the following non-limiting examples, which unless otherwise stated were performed using standard procedures, as described, for example in Maniatis et al., Molecular Cloning: A Laboratory Manual, Cold Spring Harbor Laboratory Press, Cold Spring Harbor, New York, USA (1982); Sambrook et al., Molecular Cloning: A Laboratory Manual (2 ed.), Cold Spring Harbor Laboratory Press, Cold Spring Harbor, New York, USA (1989); Davis et al., Basic Methods in Molecular Biology, Elsevier Science Publishing, Inc., New York, USA (1986); or Methods in Enzymology: Guide to Molecular Cloning Techniques Vol.152, S. L. Berger and A. R. Kimmerl Eds., Academic Press Inc., San Diego, USA (1987).

### EXAMPLES

### Example 1: Isolation of PTTG cDNA

To clarify the molecular mechanisms involved in pituitary tumorigenesis, differential display PCR was used to identify mRNAs differentially expressed in pituitary tumor cells (see, e.g., Risinger et al., 1994, Molec. Carcinogenesis, 11:13-18; and Qu et al., 1996, Nature, 380:243-247) GC and GH₄ pituitary tumor cell lines (ATCC #CCL-82 and #CCL-82.1, respectively) and an osteogenic sarcoma cell line UM108 (ATCC #CRL-1663) were grown in DMEM supplemented with 10% fetal bovine serum. Normal Sprague-Dawley rat pituitaries were freshly excised. Total RNA was extracted from tissue cultured cells and pituitary tissue using RNeasy™ kit (Qiagen) according to manufacturer's instructions. Trace DNA contamination in RNA preparations was removed by DNase1 (GenHunter Corporation) digestion. cDNA was synthesized from 200 ng total RNA using MMLV reverse transcriptase (GenHunter Corporation), and one of the three anchored primers (GenHunter Corporation). The cDNA generated was used in the PCR display.

Three downstream anchored primers AAGCT₁₁N (SEQ. ID. NO.:13; where N may be A, G, or C), were used in conjunction with 40 upstream arbitrary primers for PCR display. 120 primer pairs were used to screen mRNA expression in pituitary tumors versus normal pituitary. One tenth of the cDNA generated from the reverse transcriptase reaction was amplified using AmpliTaq DNA polymerase (Perkin Elmer) in a total volume of 20 μl containing 10 mM Tris, pH 8,4, 50 nM KCl, 1.5 mM MgCl₂, 0.001% gelatin, 2 μM dNTPs, 0.2 μM each primer and 1 μl [³⁵S]dATP. PCR cycles consisted of 30 seconds at 94° C., 2 minutes at 40° C., and 30 seconds at 72° C. for 40 cycles. The products were separated on 6% sequencing gels, and dried gels were exposed to Kodak film for 24 to 48 hours.

After development, DNA fragments amplified from pituitary tumor and normal pituitary were compared. Bands unique to pituitary tumor were excised from the gel, and DNA extracted by boiling in 100 μl water and precipitated with ethanol in the presence of glycogen (GenHunter Corporation). DNA was reamplified using the original set of primers and the same thermal cycling conditions except that the dNTP concentration was increased to 20 μM. Reaction products were run on 1% agarose gel and stained with ethidium bromide. Bands were excised from the gel, eluted (Qiagen), cloned in to TA vectors (Invitrogen) and sequenced using sequenase (USB). Using 120 primer pairs in the above-described PCR assay, 11 DNA bands that appeared to be differentially expressed in pituitary tumor cells were identified. These bands were evaluated further by Northern blot analysis, using the PCR products as probes.

For Northern blot analysis, 20 μg of total RNA were fractionated on 1% agarose gel, blotted on to nylon membrane and hybridized with random primed probe using Quickhyb solutions (Stratagene). After washing, membranes were exposed to Kodak films for 6 to 72 hours. As a result of the Northern blot assay, pituitary tumor specific signals were detected for 2 bands. DNA sequence analysis revealed that one sequence was homologous with Insulin-induced growth response protein, while the another 396 base pair fragment (amplified using 5′-AAGCTTTTTTTTTTTG-3′ [SEQ. ID. NO.:11] as the anchored primer and 5′-AAGCTTGCTGCTC-3′ [SEQ. ID. NO.:12] as an arbitrary primer) showed no homology to known sequences in the GenBank. This 396 bp fragment detected a highly expressed mRNA of about 1.3 kb in pituitary tumor cells, but not in normal pituitary nor in osteogenic sarcoma cells.

### Example 2: Characterization of cDNA Sequence Encoding PTTG

To characterize this pituitary tumor-specific mRNA further, a cDNA library was constructed using mRNA isolated from rat pituitary tumor cells. Poly A+RNA was isolated from pituitary tumor GH₄ cells using messenger RNA isolation kit (Stratagene) according to manufacturer's instructions, and was used to construct a cDNA library in ZAP Express vectors (Stratagene). The cDNA library was constructed using ZAP Express™ cDNA synthesis and Gigapack III gold cloning kit (Stratagene) following manufacturer's instructions. The library was screened using the 396 bp differentially displayed PCR product (cloned into TA vector) as the probe. After tertiary screening, positive clones were excised by in vivo excision using helper phage. The resulting pBK-CMV phagemid containing the insert was identified by Southern Blotting analysis. Unidirectional nested deletions were made into the DNA insert using EXOIII/Mung bean nuclease deletion kit (Stratagene) following manufacturer's instructions. Both strands of the insert DNA were sequenced using Sequenase (USB).

Using the 396 bp PCR fragment described in Example 1 as a probe, a cDNA clone of 974 bp (SEQ. ID. NO.:1) was isolated and characterized. This cDNA was designated as pituitary tumor-specific gene (PTTG). The sequence of PTTG contains an open reading frame for 199 amino acids (SEQ ID NO:2). The presence of an in-frame stop codon upstream of the predicted initiation codon indicates that PTTG contains the complete ORF. This was verified by demonstrating both in vitro transcription and in vitro translation of the gene product as described in Example 3.

### Example 3: In vitro Transcription and Translation of the PTTG

Sense and antisense PTTG mRNAs were in vitro transcribed using T3 and T7 RNA polymerase (Stratagene), respectively. The excess template was removed by DNase I digestion. The in vitro transcribed mRNA was translated in rabbit reticular lysate (Stratagene). Reactions were carried out at 30° C. for 60 minutes, in a total volume of 25 μl containing 3 μl in vitro transcribed RNA, 2 μl ³⁵S-Methionine (Dupond) and 20 μl lysate. Translation products were analyzed by SDS-PAGE (15% resolving gel and 5% stacking gel), and exposed to Kodak film for 16 hours.

The results indicate that translation of in vitro transcribed PTTG sense mRNA results in a protein of approximately 25 KD on SDS-PAGE, whereas no protein was generated in either the reaction without added mRNA or when PTTG antisense mRNA was utilized.

### Example 4: Northern Blot Analysis of PTTG mRNA Expression

A search of GenBank and a protein profile analysis (using a BLAST Program search of databases of the national center for Biotechnology Information) indicated that PTTG shares no homology with known sequences, and its encoded protein is highly hydrophilic, and contains no well recognized functional motifs. The tissue expression pattern of PTTG mRNA was studied by Northern Blot analysis. A rat multiple tissue Northern blot was purchased from Clontech Approximately 2 μg of poly A+RNA per lane from eight different rat tissues (heart, brain, spleen, lung, liver, skeletal muscle, kidney, and testis) was run on a denaturing formaldehyde 1.2% agarose gel, transferred to nylon membrane and UV-cross linked. The membrane was first hybridized to the full length PTTG cDNA probe, and was stripped and rehybridized to a human β-actin cDNA control probe. Hybridization was performed at 60° C. for one hour in ExpressHyb hybridization solution (Clontech). Washing was twice 15 minutes at room temperature in 2×SSC, 0.05% SDS, and twice 15 minutes at 50° C. in 0.1%SSC, 0.1% SDS. Exposure time for PTTG probe was 24 hrs, and actin probe 2 hours.

The results of the Northern assay indicate that testis is the only tissue, other than pituitary tumor cells, that expresses PTTG mRNA, and the testis expression level is much lower (2 μg polyA+mRNA, 24 hour exposure) than in pituitary tumor cells (20 μg total RNA, 6 hour exposure). Interestingly, the testicular transcript (about 1 Kb) is shorter than the transcript in pituitary tumors (1.3 Kb), indicating that the mRNA is differentially spliced in testis, and that the 1.3 Kb transcript is specific for pituitary tumor cells.

### Example 5: Over-expression of PTTG in NIH 3T3 Fibroblast Cells

Since PTTG mRNA is over-expressed in pituitary tumor cells, whether this protein exerts an effect on cell proliferation and transformation was determined. An eukaryotic expression vector containing the entire coding region of PTTG was stably transfected into NIH 3T3 fibroblasts.

The entire coding region of the PTTG was cloned in frame into pBK-CMV eukaryotic expression vector (Stratagene), and transfected into NIH 3T3 cells by calcium precipitation. 48 hrs after transfection, cells were diluted 1:10 and grown in selection medium containing 1 mg/ml G418 for two weeks in when individual clones were isolated. Cell extracts were prepared from each colony and separated on 15% SDS-polyacrylamide gels, and blotted onto nylon membrane. A polyclonal antibody was generated using the first 17 amino acids of PTTG as epitope (Research Genetics). The antibody was diluted 1:5000 and incubated with the above membrane at room temperature for 1 hour. After washing, the membrane was incubated with horseradish peroxidase-labeled secondary antibody for one hour at room temperature. The hybridization signal was detected by enhanced chemiluminescence (ECL detection system, Amersham).

Expression levels of the PTTG were monitored by immunoblot analysis using the above-described specific polyclonal antibody directed against the first 17 amino acids of the protein. Expression levels of individual clones varied, and clones that expressed higher protein levels were used for further analysis.

### Example 6: Effect of PTTG Expression on Cell Proliferation

A non-radioactive cell proliferation assay was used to determine the effect of PTTG protein over-expression on cell proliferation (see, e.g., Mosmann, T., 1983, J. Immunol. Meth., 65:55-63; and Carmichael et al., 1987, Cancer Res., 47:943-946). Cell proliferation was assayed using CellTiter 96TM Non-radioactive cell proliferation assay kit (Promega) according to the manufacturer's instructions. Five thousand cells were seeded in 96 well plates (6 wells for each clone in each assay), and incubated at 37° C. for 24 to 72 hours. At each time point, 15 μl of the Dye solution were added to each well, and incubated at 37° C. for 4 hours. One hundred μl of the solubilization/stop solution were then added to each well. After one hour incubation, the contents of the wells were mixed, and absorbance at 595 nm was recorded using an ELISA reader. Absorbance at 595 nm correlates directly with the number of cells in each well.

Three independent experiments were performed. The cell growth rate of 3T3 cells expressing PTTG protein (assayed by cellular conversion of tetrazolium into formazan) was suppressed 25 to 50% as compared with 3T3 cells expressing the pCMV vector alone, indicating that PTTG protein inhibits cell proliferation (data not shown).

### Example 7: PTTG Induction of Morphological Transformation and Soft-agar Growth of NIH 3T3 Cells

The transforming property of PTTG protein was demonstrated by its ability to form foci in manslayer cultures and show anchorage-independent growth in soft agar (Table 1). As primary pituitary cells are an admixure of multiple cell types and they do not replicate in vitro, NIH 3T3 cells were employed. For the soft agar assay (Schwab et al., 1985, Nature, 316:160-162), 60 mM tissue culture plates were coated with 5 ml soft-agar (20% 2×DEEM, 50% DEEM, 10% fetal bovine serum, 20% 2.5% agar, melted and combined at 45° C.). 2 ml cells suspended in medium were then combined with 4 ml agar mixture, and 1.5 ml of this mixture added to each plate. Cells were plated at a density of 10⁴ cells/dish and incubated for 14 days before counting the number of colonies and photography. Only colonies consisting of at least 40 cells were counted. Values shown in Table 1 are means±SEM of triplicates.

TABLE 13
Colony Formation by NIH 3T3 Cells
Transfected with PTTG cDNA Constructs

| | Growth in | Efficiency of Colony |
|---|---|---|
| Cell line | Soft Agar | formation in Soft Agar (%)* |
| No DNA | 0 | 0 |
| Vector only | 1.3 ± 0.7 | 0.013 |
| PTTG 3 | 26 ± 4.6 | 0.26 |
| PTTG 4 | 132 ± 26 | 1.32 |
| PTTG 8 | 33 ± 6.0 | 0.33 |
| PTTG 9 | 72 ± 13 | 0.72 |
| PTTG 10 | 92 ± 18 | 0.92 |

*Efficiency of colony formation was calculated as percentage of number of colonies divided by total number of cells.

The results indicate that NIH 3T3 parental cells and 3T3 cells transfected with pCMV vector do not form colonies on soft agar, whereas 3T3 cells transfected with PTTG form large colonies. In addition, focal transformation is observed in cells over-expressing PTTG protein, but cells expressing pCMV vector without the PTTG insert showed similar morphology to the parental 3T3 cells.

### Example 8: Assay to Determine Whether PTTG is Tumorigenic in Vivo

To determine whether PTTG is tumorigenic in vivo, PTTG-transfected 3T3 cells were injected subcutaneously into athymic nude mice. 3×10⁵ cells of either PTTG or pCMV vector-only transfected cells were resuspended in PBS and injected subcutaneously into nude mice (5 for each group). Tumors were excised from sacrificed animals at the end of the 3rd week and weighed. All injected animals developed large tumors (1-3 grams) within 3 weeks. The results are shown in Table 14 below. No mouse injected with vector-only transfected cells developed tumors. These results clearly indicate that PTTG is a potent transforming gene in vivo.

TABLE 14
In vivo Tumorigenesis by NIH 3T3 Cells
Transfected with PTTG cDNA Expression Vector

| Cell line | No. Animals injected | Tumor formation |
|---|---|---|
| Vector only | 5 | 0/5 |
| PTTG 4 | 5 | 5/5 |

### Example 9: Human Carcinoma Cell Lines Express PTTG

The pattern of expression of PTTG in various human cell lines was studied employing a multiple human cancer cell line Northern blot (Clontech). The specific cell lines tested are shown in Table 15 below.

TABLE 15
Human Carcinoma Cell Lines Tested

| | Cell Line | PTTG Expression |
|---|---|---|
| 1 | Promyelocytic Leukemia HL-60 | + |
| 2 | HeLa Cell S3 | + |
| 3 | Chronic Myelogenous Leukemia K-562 | + |
| 4 | Lymphoblastic Leukemia MOLT-4 | + |
| 5 | Burkitt's lymphoma Raji | + |
| 6 | Colorectal Adenocarcinoma SW 480 | + |
| 7 | Lung Carcinoma A549 | + |
| 8 | Melanoma G361 | + |

About 2 μg polyA RNA from each of the 8 cell lines indicated in Table 3 above were placed on each lane of a denaturing formaldehyde 1.2% agarose gel, separated by denaturing gel electrophoresis to ensure intactness, transferred to a charge-modified nylon membrane by Northern blotting, and fixed by UV irradiation. Lanes 1 to 8 contained RNA from promyelocytic leukemia HL-60, HeLa cell line S3, human chronic myelogenous leukemia K-562, lymphoblastic leukemia MOLT-4, Burkitt's lymphoma Raji, colorectal adenocarcinoma SW 480, lung carcinoma A549 and melanoma G361, respectively. RNA size marker lines at 9.5, 7.5, 4.4, 2.4, and 1.35 kb were indicated in ink on the left margin of the blot, and utilized as sizing standards, and a notch was cut out from the lower left hand corner of the membrane to provide orientation. Radiolabeled human β-actin cDNA was utilized as a control probe for matching of different batches of polyA RNAs. A single control band at 2.0 kb in all lanes spotted is confirmatory.

The blots were probed with the full length rat PTTG cDNA probe (SEQ. ID NO:1; 974 bp) at 60° C. for 1 hr. in ExpressHyb hybridization solution (Clontech) as described by Sambrook et al., the relevant section of which reference is incorporated herein by reference. See, Sambrook et al., Molecular Cloning: A Laboratory Manual, 2nd. Ed, Cold Spring Harbor Laboratory Press, Cold Spring Harbor, N.Y. (1989). The blots were then washed twice for 15 min at room temperature in 2×SSC, 0.05% SDS, and twice for 15 min at 50° C. in 0.1% SSC, 0.1% SDS. A more detailed description of the remaining experimental procedures masy be found in Pei & Melmed, the relevant section of which is incorporated herein by reference. (See, Pei & Melmed, Endocrinology 4: 433-441 [1997]).

All cells tested by the Northern blot analysis as described above evidenced expression of human PTTG (i.e., PTTG1), including lymphoma, leukemia, melanoma and lung carcinomas, among others.

### Example 10: Molecular Cloning of Human PTTG cDNA

A human fetal liver cDNA library (Clontech, Palo Alto, Calif.) was screened as described by Maniatis et al. (Maniatis et al, Molecular cloning, Cold Spring Harbor Press, 1989), using a radioactively labeled cDNA fragment of the entire rat PTTG coding region as a probe. The cDNA inserts from positive clones were subcloned into plasmid pBluescript-SK (Stratagene, La Jolla, Calif.), and subjected to sequence analysis using Sequenase kit (U.S. Biochemical Corp., Cleveland, Ohio).

A complete open reading frame containing 606 bp was found in the positive clones. The homology between the nucleotide sequences of the open reading frame and the coding region of rat PTTG is 85%. Amino acid sequence comparison between the translated product of this open reading frame and rat PTTG protein reveals 77% identity and 89% homology. The cDNAs obtained from these clones represents human homologies of rat PTTG. No other cDNA fragments with higher homology were detected from the library.

### Example 11: Tissue Distribution of Human PTTG mRNA

Total RNA was prepared using Trizol Reagent (Gibco-BRL, Gaithersburg, Md.) from normal human pituitary glands (Zoion Research Inc. Worcester, Mass.) and fresh human pituitary tumors collected at surgery and frozen in liquid nitrogen. 20 mg total RNA were used for 1% agarose gel electrophoresis. RNA blots (Clontech, Palo Alto, Calif.) derived from normal adult and fetal tissues as well as from malignant tumor cell lines, were hybridized with radioactively labeled human cDNA fragment containing the complete coding region. The RNA isolated from each cell line was transferred onto a nylon membrane (Amersham, Arlington Heights, Ill.), and hybridized with radioactively labeled probe at 55° C. overnight in 6×SSC, 2×Denhardt's solution, 0.25% SDS. The membranes were washed twice at room temperature for 15 minutes each, and then for 20 minutes at 60° C. in 0.5×SSC, 0.1% SDS, and autoradiographed. The autoradiography was carried out using Kodak BIOMEX-MR film (Eastman Kodak, Rochester, N.Y.) with an intensifying screen. The blots were stripped by washing for 20 minutes in distilled water at 95° C. for subsequent probing.

The results from the Northern blot analysis indicated that PTTG is expressed in liver, but not in brain, lung, and kidney of human fetal tissue. In addition, PTTG is strongly expressed in testis, modestly expressed in thymus, and weakly expressed in colon and small intestine of normasl human adult tissue. No expression was detected by Northern analysis in brain, heart, liver, lung, muscle, ovary, placenta, kidney, and pancreas.

The expression of PTTG in several human carcinoma cell lines was also analyzed by Northern blots. In every carcinoma cells examined, PTTG was found highly expressed. The human tumor cell lines tested are listed in Table 16 below.

TABLE 16
Tested Human Tumor Cell Lines

Promyelocytic leukemia HL-60
Epitheloid carcinoma HeLa cell S3
Chronic myelogenous leukemia K-562
Lymphoblastic leukemia MOLT-4
Burkitt's lymphoma Raji
Colorectal adenocarcinoma SW 480
Lung carcinoma A549
Melanoma G361
Hepatocellular carcinoma Hep 3B
Thyroid carcinoma TC-1
Breast adenocarcinoma MCF-7
Osteogenic sarcoma U2 OS
Placenta choriocarcinoma JAR
Choriocarcinoma JEG-3

### Example 12: Human PTTG Expression in Normal Pituitary and Pituitary Tumors

RT-PCR was performed as follows. 5 mg total RNA were treated with 100 U RNase-free DNase I at room temperature for 15 minutes. DNase I was inactivated by incubation at 65° C. for 15 minutes. The sample was then used for reverse transcription using oligo-dT primer and SuperScript II reverse transcriptase (Gibco-BRL, Gaithersburg, Md.). After reverse transcription, the sample was subjected to PCR amplification with PCR SuperMix (Gibco-BRL, Gaithersburg, Md.) using hPTTG-specific primers and human cyclophilin A-specific primers as an internal control.

Northern blot analysis indicated that the level of expression of PTTG is quite low in normal pituitary as well as in pituitary tumors. Therefore, comparative RT-PCR was used to study the expression of PTTG quantitatively in normal pituitary and pituitary tumors. The results of this study showed that in most of pituitary tumors tested, including non-functioning tumors, GH-secreting tumors, and prolactinomas, the expression level ofPTTG was higher than that of normal pituitary.

### Example 13: Stable Transfection of Human PTTG into NIH 3T3 Cells

The complete coding region of hPTTG cDNA was subcloned in reading frame into the mammalian expression vector pBK-CMV (Stratagene, La Jolla, Calif.), and transfected into NIH 3T3 fibroblast cells by Lipofectamine (Gibco-BRL, Gaithersburg, Md.) according to manufacturer's protocol. 24 hours after transfection, the cells were serially diluted and grown in selection medium containing 1 mg/ml G418 for 2 weeks. Individual clones were isolated and maintained in selection medium. Total RNA was isolated from hPTTG-transfected cell lines as well as from control cells in which blank vector pBK-CMV had been transfected. Northern blot was performed to confirm overexpression of hPTTG in transfected cell lines. These cell lines were used in subsequent cell proliferation assay as well as in vitro and in vivo transformation assay.

### Example 14: Cell Proliferation Assay

A cell proliferation assay was performed using the CellTiter 96 non-radioactive cell proliferation assay kit (Promega Medicine, WI) according to the manufacturer's protocol. 5,000 cells were seeded in 96-well plates and incubated at 37° C. for 24-72 hours. Eight wells were used for each clone in each assay. At each time point, 15 ml of dye solution was added to each well and the cells were incubated at 37° C. for 4 hours. After incubation, 100 ml solubilization/stop solution were added to each well, and the plates incubated overnight at room temperature. The absorbance was determined at 595 nm using an ELISA plate reader.

Control and hPTTG-overexpressing NIH 3T3 cells were used to perform this assay. The results indicated that the growth of cells transfected with the PTTG-expressing vector was suppressed by 30˜45% as compared with cells transfected with blank vector. These results clearly show that the PTTG protein inhibits cell proliferation.

### Example 15: In Vitro and In Vivo Transformation Assay

### (a) In vitro Transformation Assay

Control and hPTTG-transfected cells were tested for anchorage-independent growth in soft agar. 3 ml of soft agar (20% of 2×DMEM, 50% DMEM, 10% fetal bovine serum, and 20% of 2.5% agar, melted and mixed at 45° C.) were added to 35mm tissue dishes. 10,000 cells were mixed with 1 ml soft agar and added to each dish, and incubated for 2 weeks until colonies could be counted and photographed.

### (b) In Vivo Transformation Assay

5×10⁵ cells containing either a blank vector or hPTTG-expressing cells were injected into nude mice. The mice were sacrificed two weeks after injection, and the tumors formed near the injection sites examined.

When the NIH 3T3 cells stably transfected with the PTTG-expressing vector were tested in an anchorage-independent growth assay, these cells caused large colony formation on soft agar, suggesting the transforming ability of PTTG protein.

When the NIH 3T3 cells were injected into nude mice, they caused in vivo tumor formation within 2 weeks after injection. These data indicate that human PTTG, as its rat homologue, is a potent transforming gene.

### Example 16: Inhibition of Cell Transformation/Tumor Formation by PTTG C-Terminal Polypeptide

Cell lines. NIH 3T3 cells were maintained in high glucose (4.5 g/L) DMEM (Gibco-BRL) supplemented with 10% fetal bovine serum. HeLa cells were maintained in low glucose (1 g/L) DMEM (Gibco-BRL) supplemented with 10% fetal bovine serum (FBS). T-47D and MCF-7 cells were maintained in high glucose DMEM (Gibco-BRL) supplemented with 10% fetal bovine serum and 0.01 mg/mL bovine insulin (Sigma). All cell lines were obtained from American Type Culture Collection (ATCC).

Site-directed Mutagenesis and Stable Transfection of Human and Mutant PTTG into NIH 3T3 Cells.

Point mutations on the proline-rich domain(s) of wild type human PTTG polypeptide (wtPTTG) were generated by PCR-based site-directed mutagenesis. Two synthetic oligonucleotides, 5′-GATGCTCTCCGCACTCTGGGAATCCAATCTG-3′ (SEQ. ID. NO.:5) and 5′-TTCACAAGTTGAGGGGCGCCCAGCTGAAACAG-3′ (SEQ. ID. NO.:6), which cause point mutations that result in amino acid sequence changes P163A, S165Q, P166L, P170L, P172A, and P173L in the wtPTTG protein, were used to amplify human PTTG cDNA cloned into pBlue-Script-SK vector (Stratagene). Amplified mutated cDNA (mutPTTG) was then cloned into mammalian expression vector pCI-neo (Promega). Overexpression of mutPTTG in transfected cells was confirmed by Northern analysis and RT-PCR followed by direct sequence analysis. wtPTTG and mutPTTG were subcloned into pCl-neo, and the vector was used to transfect NIH 3T3 cells as described in Zhang, X., et al. [1999a].

Transactivation assay. wtPTTG cDNA was fused in frame with pGAL4 (Stratagene), designated pGAL4-wtPTTG and was used as template for deletion and mutation analysis; mutPTTG cDNA was also fused in frame with pGAL4 and designated pGAL4-mutPTTG. pGAL4-VP 16 was used as a positive control. Experimental plasmids; were co-transfected with pLUC and pCMV-β-Gal (as internal control). Cell lysates were prepared 48 hours after transfection and assayed for luciferase activity as described (Wang, Z. and Melmed, S. [2000]; Sambrook, J., et al., Molecular Cloning: A Laboratory Manual, 2d Ed. Cold Spring Harbor Laboratory, Cold Spring Harbor, N.Y., 10.2.1-10.2.6 [1989]).

Constructions of expression vectors for wild type and mutant human PTTG C-terminal polypeptides. To generate wtPTTG and mutPTTG C-terminal polypeptide expression vector, the internai Xba I site of wtPTTG and mutPTTG cDNA and the 3′-portions of these cDNAs were cloned into pCI-neo (Promega, Madison, Wis.) via Xba I and Not I sites. In these clone, the ATG for M147 of full-length PTTG is used as an initiation codon, generating a polypeptide of 56 amino acid residues corresponding to nucleotide positions 147 through 202 of full-length wtPTTG.

Stable transfection of human PTTG C-terminal peptide into tumor cells. Wild type and mutant PTTG C-terminal expression constructs were transfected into HeLa, MCF-7, and T47-D cells with Lipofectin (GIBCO-BRL) according to the manufacturer's protocol. Twenty-four hours after transfection, cells were serially diluted and selected with G418 (1 mg/mL) for 2 weeks. Individual clones were isolated and maintained in selection medium (respective high or low glucose DMEM with 10% FBS, as described above, and G148 [1 mg/mL]), and total RNA was extracted from transfected cells. Expression of wild-type and mutated PTTG -C terminal was confirmed by RT-PCR using two synthetic oligonucleotides, with one specific to the 5′-nontranslational region from vector pCI-neo, 5′-GGCTAGAGTACTTAATACGACTCACTATAGGC-3′ (SEQ. ID. NO.:7), and the other to the 3′-translational region of PTTG1 cDNA, 5′-CTATGTCACAGCAAACAGGTGGCAATTCAAC-3′ (SEQ. ID. NO.:8), followed by direct sequence analysis.

In vitro colony formation and in vivo tumorigenesis. NIH 3T3 stable transfectants were tested in vivo as described in Zhang, X., et al. [1999a]. Transfected cells were tested for anchorage-independent growth in soft agar as described Zhang, X., et al. [1999a]. HeLa cells were incubated for 3 weeks and MCF-7 (breast carcinoma) and T-47D (breast carcinoma) cells for 2 weeks. For in vivo assays of tumorigenesis, 1×10⁷ MCF-7 stable transfectants were resuspended in 500 μL MATRIGEL basement membrane matrix (Becton Dickinson, Bedford, Mass.) and were injected subcutaneously into nude mice (three mice for each group). After four weeks, animals were photographed and tumors were excised and weighed.

ELISA of basic fibroblast growth factor (bFGF) in conditioned medium. The concentration of basic fibroblast growth factor (bFGF) concentration in HeLa cell culture medium was assayed using Quantikine HS Human FGF Basic Immunoassay Kit (R&D Systems, Minneapolis, Minn.) according to the manufacturer's protocol. Cells (1×10⁵) were plated in 100-mm cell culture dishes. After 72 hours, the culture medium was collected and 200 mL was used for ELISA assay.

Effects of wild type human PTTG and mutant PTTG overexpression on tumor induction. It was previously demonstrated that NIH 3T3 cells overexpressing wild type PTTG formed large colonies in an anchorage-independent growth assay and formed tumors when injected into athymic nude mice, while point mutations in the proline-rich region (P163A, P170L, P172A, and P173L) abrogated formation of colonies and tumors (Zhang, X., et al. [1999a]). Overexpression of wtPTTG and mutPTTG (P163A, S165Q, P166L, P170L, P172A, and P173L) in each transfectant cell line was confirmed by Northern analysis and RT-PCR followed by direct sequence analysis (not shown).

It was further shown that overexpressing PTTG transfectants injected into athymic nude mice caused tumor formation within 2 weeks in all injected animals. Five mice in each of three groups were injected subcutaneously with 3×10⁵ NIH 3T3 cells transfected with: (1) control cell line (transfected with pGAL4 vector alone); (2) wild type PTTG-overexpressing (wtPTTG); or (3) mutant PTTG-overexpressing (mutPTTG [P163A, S 165Q, P166L, P170L, P172A, and P173L]). After 2 weeks, mice were sacrificed and tumors were excised and weighed. In the mice injected with control transfectants or mutPTTG transfectants, no tumors developed, but mice injected with transfectant cells bearing wtPTTG developed tumors without exception. Tumor weights ranged from 470 to 1500 mg (Table 17).

TABLE 17
Tumor formation by PTTG-expressing
NIH 3T3 Cells in Athymic Nude Mice

| | Tumor weight (mg) | |
|---|---|---|
| Vector | wtPTTG | mutPTTG |
| none* | 1500 | none |
| none | 770 | none |
| none | 1250 | none |
| none | 550 | none |
| none | 470 | none |

*none = no detectable tumor.

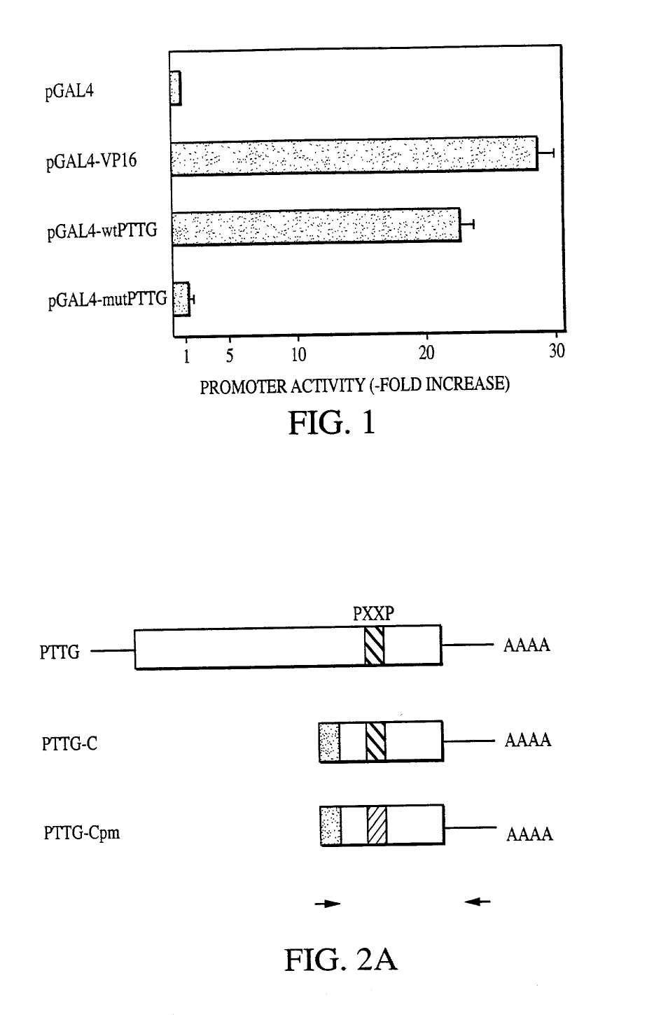

PTTG exhibits transcriptional activation. Vector pGal4 alone (negative control) did not activate the luciferase (luc) reporter, and a known activation domain, VP16, significantly increased reporter activity about 28-fold. pGAL4-wtPTTG exhibited transactivation properties and induced reporter activity about 22-fold (FIG. 1).

Transcriptional activity of pGAL4-mutPTTG (mutated proline region [P163A, S 165Q, P166L., P170L, P172A, and P173L]), other point mutations, as well as a separate deletions (d) of wtPTTG were also tested as indicated in Table 18. In Table 18, the indicated plasmids were co-transfected with pLuc and pCMV-β-Gal into NIH 3T3 cells, and luciferase assays were performed, with β-Gal serving as the internal control. Each value represents triplicate wells from two independent experiments (±SEM); transactivation by wtPTTG was designated 100%. pGAL4-mutPTTG exhibited about 95% transactivating activity compared to pGAL4-wtPTTG, thus confirming the importance of the wtPTTG proline-rich motif for transactivation.

TABLE 18
Transactivation assay of hPTTG mutants.

| Mutant | activation activity (%)(±SEM) |
|---|---|
| pGAL4-wtPTTG | 100 |
| 163 Pro → Ala | 100 ± 10 |
| 166 Pro → Ala | 45 ± 5* |
| 170 Pro → Ala | 100 ± 10 |
| 182 Pro → Ala | 100 ± 10 |
| 152 Glu → Gln | 100 ± 10 |
| 192 Glu → Gln | 50 ± 3* |
| 165 Ser → Ala | 30 ± 3* |
| 165 Ser → Leu | 20 ± 2* |
| 176 Ser → Ala | 100 ± 10 |
| 183 Ser → Ala | 100 ± 10 |
| 184 Ser → Ala | 100 ± 10 |
| d(1-100) | 100 ± 10 |
| d(180-202) | 100 ± 10 |
| mutPTTG | 6 ± 1* |

*p < 0.01

Human PTTG C-terminal peptide expression blocks cell transformation. The critical role of the proline-rich region in transactivation, transformation and tumor formation, as described above, implies that PTTG functions through SH3-mediated signal transduction. If human PTTG1 protein mediates the SH3-related signal cascade, it probably contains at least two functional domains interacting with upstream and downstream signal molecule(s), respectively. A mutant protein containing only one such functional domain could then act in a dominant-negative manner to abrogate wild-type protein function and disrupt signal transduction.

Based on this hypothesis, a truncated PTTG1 mutant peptide, lacking N-terminal amino acid residues 1-146, was introduced into human carcinoma cells. An expression construct was used expressing a PTTG-C peptide corresponding to residues 147-202 of the full-length protein, under the control of a CMV promoter. This polypeptide contains the proline-rich domain(s) (residues 163-173; Zhang, X., et al. [1999a]), and when the coding sequence was fused to glutathione S-transferase (GST), it was expressed in Escherichia coli as an intact protein with the appropriate molecular weight (data not shown). Mutant expression vector pCIneo-mutPTTG (mutated proline region [P163A, S165Q, P166L., P170L, P172A, and P173L]), as well as the empty vector pCI-neo alone as control, were stably transfected into HeLa, MCF-7, and T-47D human carcinoma cell lines.

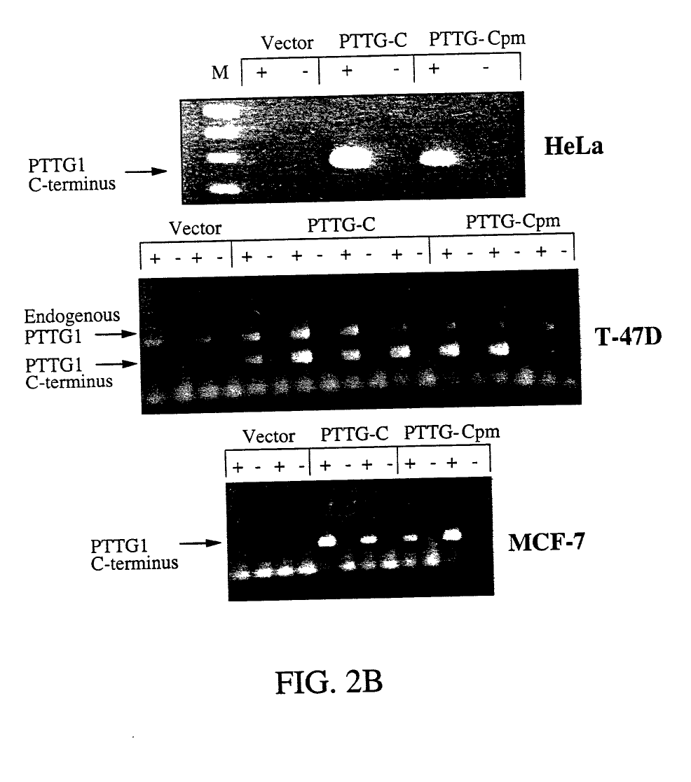

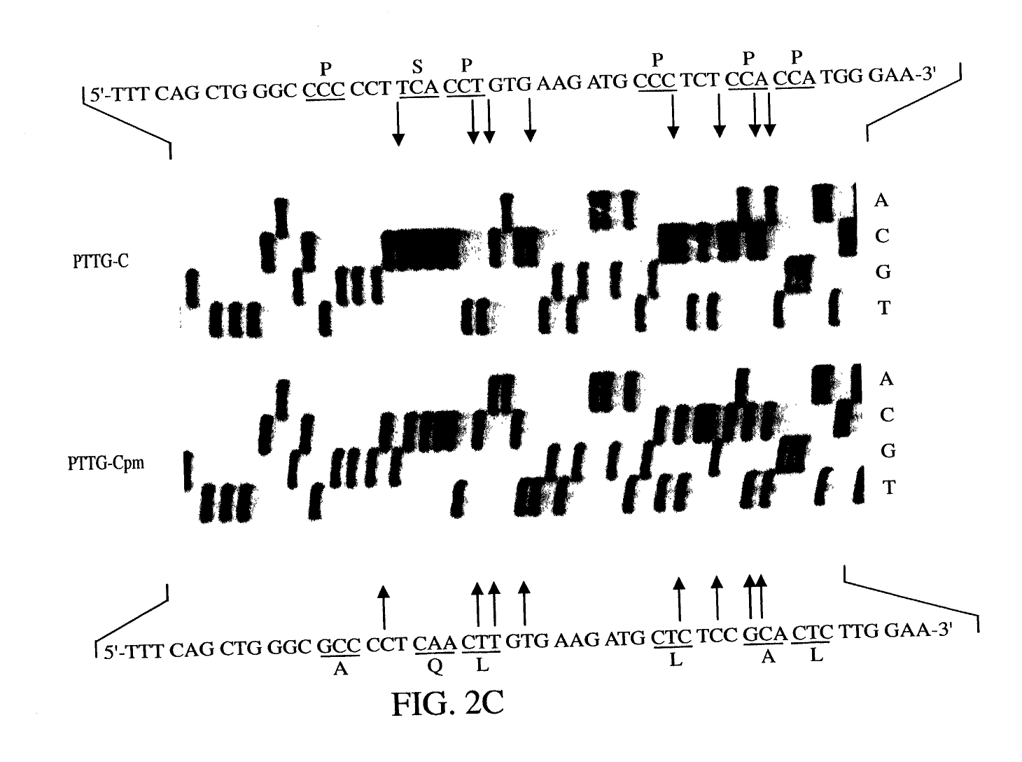

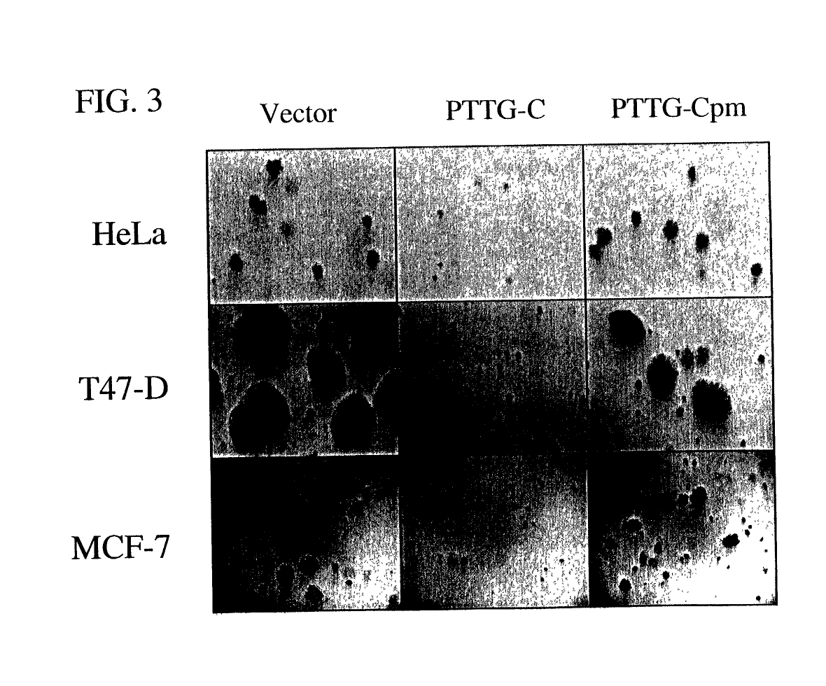

Transfectants expressing wild-type PTTG carboxy-terminal peptide (PTTG- C), PTTG C-terminal mutated in several proline residues (PTTG-Cpm; mutated proline region [P163A, S165Q, P166L., P170L, P172A, and P173L]), and vector (V), were isolated. Expression of each transfectant line was confirmed by RT-PCR, using a primer directed to the 5′-nontranslational region of the expression vector and a primer directed to the 3′-translational region of PTTG mRNA, followed by direct sequence analysis (FIGS. 2A, 2B, and 2C). Transforming abilities of all three of these stably transfected cell lines were tested in an anchorage-independent growth assay, PTTG-Cpm cells were observed to form large colonies, as did control V cells containing the same expression vector but lacking either wild type or mutant C-terminal polypeptide. Each transfectant cell line was plated in three different plates. HeLa was scored on the 21st day and T-47D and MCF-7 on the 14th day. Colonies consisting of 60 or more cells were scored However, the number and size, of colonies formed by cells expressing PTTG-C were markedly reduced (p<0.01) (FIG. 3). Table 19 (below) summarizes the soft agar colony formation for each cancer cell type.

TABLE 19
Colony Formation by PTTG I C-terminal (PTTG-C)
and mutant PTTG C-terminal (PTTG-Cpm) Expressing
Cells in Soft Agar.

| | | Colonies/10⁴ Cells |
|---|---|---|
| Cell Line | Vector | (mean ± SEM) |
| HeLa | Vector alone | 1465 ± 54 |
| | Vector alone | 2392 ± 55 |
| | PTTG-C | 11 ± 2* |
| | PTTG-C | 6 ± 1* |
| | PTTG-C | 48 ± 3* |
| | PTTG-C | 3 ± 1* |
| | PTTG-Cpm | 1169 ± 77 |
| | PTTG-Cpm | 1097 ± 79 |
| | PITG-Cpm | 2615 ± 76 |
| T-47D | Vector alone | 135 ± 4 |
| | PTTG-C | 46 ± 5* |
| | PTTG-C | 52 ± 2* |
| | PTTG-Cpm | 193 ± 5 |
| | PTTG-Cpm | 106 ± 5 |
| MCF-7 | Vector alone | 287 ± 3 |
| | PTTG-C | 9 ± 3* |
| | PTTG-C | 34 ± 4* |
| | PTTG-Cpm | 236 ± 11 |
| | PTTG-Cpm | 206 ± 4 |

*P < 0.01

Human PTTG C-terminal polypeptide-expressing MCF-7 breast carcinoma cells fail to develop tumors in vivo. Stably transfected MCF-7 breast carcinoma cell lines were injected (1×10⁷ cells/per mouse in 500 μL MATRIGEL basement membrane matrix) subcutaneously into athymic nude mice. After four weeks, mice were photographed, killed, and their tumors were excised and weighed. Three mice injected with cells transfected with control vector only developed visible tumors in 4 weeks, while three mice injected with PTTG-C-transfected cells failed to generate tumors. At autopsy, absence of subcutaneous or other peripheral tumor formation was confirmed in the mice receiving PTTG-C transfected cells. Three mice injected with PTTG-Cpm transfected cells also developed tumors after 4 weeks, which were similar in size to those developed in mice injected with control vector-transfected cells, indicating that the mutated PTTG-C-terminal polypeptide lost its ability to abrogate endogenous PTTG function (Table 20).

TABLE 20
Tumor formation by PTTG-C expressing MCF-7
Breast Carcinoma Cells in Athymic Nude Mice.

| | Tumor weight (mg) | |
|---|---|---|
| Vector | PTTG-C | PTTG-Cpm |
| 212 | none* | 185 |
| 235 | none | 196 |
| 209 | none | 203 |

*none = no detectable tumor

These results show that overexpression of the PTTG C-terminal peptide caused cancer cells to lose their abilities for in vitro cell transformation and ex vivo tumor growth. Also, the importance of proline-rich regions is further confirmed here, since PTTG C-terminal peptide containing point mutations of these proline residues failed to interfere with transforming activity or tumor-forming activity in vivo.

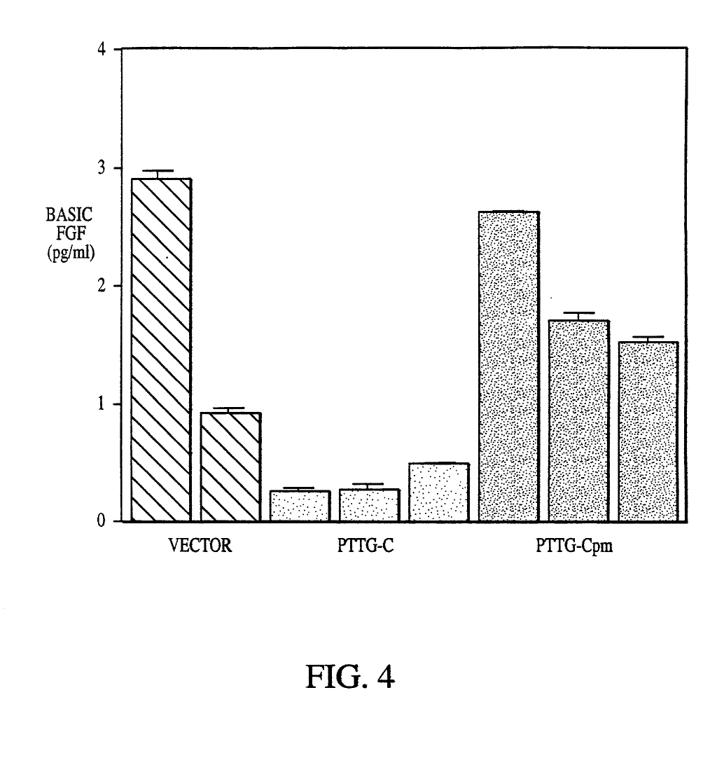

Suppression of bFGF secretion and PRL expression by PTTG-C peptide. As cells expressing wild-type human PTTG-C terminal peptide had markedly reduced colony forming ability on soft agar and were also unable to induce solid tumor growth in vivo, expression of bFGF was tested in HeLa transfectants. An enzyme-linked immunoabsorbent assay (ELISA) was performed to examine bFGF levels in conditioned medium derived from 72-hour cultures of HeLa transfectants. As shown in FIG. 4, bFGF levels were markedly decreased in conditioned medium derived from PTTG-C DNA-transfected cells than those derived from vector-only and PTTG-Cpm-transfected cells, indicated a suppression of bFGF secretion resulting from the presence of PTTG carboxy-terminal peptide.

Since, the growth rate of solid tumors is directly related to activation of angiogenesis and recruitment of new blood vessels, this shows that, in accordance with the inventive method, the ability for new blood vessel growth can be impaired by the inventive PTTG-C peptides, providing an additional mechanism leading to the failure of in vivo neoplastic cellular proliferation and tumor growth. Experimental tumors do not grow more than 1 or 2 mm in diameter in the absence of angiogenesis. (Folkman, J., N. Engl. J. Med. 285:1182-1186 [1971]; Folkman, J., and Klagsburn, M. (1987) Science 235:442-447 [1987]). The human cancer cell lines used in this study form prominent solid tumors (>2 mm in diameter) indicating active angiogenesis.

Moreover, these results imply that additional hormonal regulatory cascades can be affected by the inventive PTTG-C peptides, because reduced bFGF secretion can result in altered expression of bFGF-mediated pathways, for example prolactin (PRL) expression. For example, expression of the same human wild-type PTTGC-terminal peptide (amino acid residues 147-202 of SEQ. ID. NO.:4) in rat prolactin (PRL)- and growth hormone (GH)-secreting GH3 cells caused markedly reduced PRL promoter activity (about 16-fold decrease), PRL mRNA expression (about 10-fold decrease), and prolactin protein expression (about 72-fold decrease) in comparison to rat GH3 cells transfected with control vector alone or GH3 cells expressing a mutated PTTG1 C-terminal fragment (P163A, S 165Q, P166L, P170A, P172A, and P173L; data not shown). Furthermore, a compensatory increase in GH mRNA (about 13-fold increase) and protein (about 37-fold increase) were observed in the PTTG-C-terminal expressing GH3 cells. These observations demonstrate that PTTG carboxy-terminal peptide expressed in GH3 cells alters the hormonal secretory pattern by silencing PRL-gene expression and augmenting GH expression.

### Example 17: Expression of PTTG in Normal and Leukemic T-Lymphocyte Cells

Cell culture. T-lymphocytes were prepared by positive selection of mononuclear cells from fresh peripheral venous blood of healthy human adults. In some experiments leukopack preparations of American Red Cross anonymous donors were used. Mononuclear blood cells were isolated by gradient centrifugation using Lymphoprep™ KIT (Nycomed Pharma AS, Oslo, Norway). When leukopack preparations were used, the isolated mononuclear cells were first frozen in 90% fetal bovine serum (FBS) supplemented with 10% DMSO. These cells were thawed and washed and grown in RPMI-1640 medium supplemented with 10% FBS. Human Jurkat leukemia T cell and human HL-60 promyelocytic leukemia cells were grown in the same medium.

Double T cell selection was performed using immunomagnetic beads (Dynal CD2 TM CELLectionTM KIT, Dynal AS, Oslo, Norway), and cells were further activated by culturing in Petri dishes containing immobilized anti-human monoclonal CD3 antibody (PharMingen International, Becton Dickinson Co.). Antibody immobilization was achieved by 90 min incubation (37° C.) of fresh 60 mm plastic Petri dishes filled with 1 mL of anti-CD3 solution (10 μg/mL) in phosphate buffered saline (PBS). KIT protocols provided by the manufacturers were used during isolation and activation of T-cells. In some experiments, resting T-cells were activated using phytohemagglutinin (PHA, 5 μg/mL, Gibco-BRL). In each experiment, cells were labeled with triple fluorochrome-labeled anti-CD3/CD19/CD45 antibodies (Caltag Laboratories, Inc., Burlingame, Calif.) and then were studied using flow cytometry (Becton-Dickinson FACScan) in order to check relative amounts of anti-CD3-labeled T-lymphocytes in the cell population. Samples containing 95-97% of T cells and 0-0.05% of B cells were used in the experiments. Cells were also treated with sodium salt of hydrocortisone 21-hemisuccinate (0.1-1 μM, Sigma, St.Louis, Mo.), cyclosporin A (1 μg/mL, Calbiochem, SanDiego, Calif.), TGF betal (10 ng/mL, R & D Systems, Inc., Minneapolis, Minn.), aphidicolin (1 μg/mL, Calbiochem), nocodazole (500 ng/mL, Calbiochem) beginning from zero time after the starting of cell activation. Control experiments for dissolvants (0.1% C₂H₅OH (aphidicolin) and 0.2% DMSO (nocodazole)) did not evidence an effect on PTTG mRNA expression.

Northern blot analysis. Total RNA was isolated using TRIzol Reagent (Gibco-BRL, Grand Island, N.Y.); RNA (about 5 μg for T cells and 20 μg for Jurkat cells) was electrophoresed in 1% agarose-formaldehyde gel and blotted onto Hybond-N membranes (Amersham International, UK). The membranes were UV-cross-linked, prehybridized for 1 hour at 68° C. in QuickHyb solution (Stratagen, La Jolla, Calif.), and then were hybridized for 3 hours at 68° C. in the same solution supplemented with random-primed ³²P-labeled human PTTG cDNA (2×10⁶ cpm/mL) and sonicated and denaturated salmon sperm DNA (Stratagen). When IL-2 mRNA expression was studied, sequences of oligonucleotide sense and anti-sense primers for PCR preparation of IL-2 cDNA probe (sized 457 base pairs) were as previously reported. (Butch, A W et al., Cytokine expression by germinal center cells, J Immunol. 150:39-47 [1993]). Cyclophilin cDNA probe was from Ambion, Inc. (Austin, Tex.). The membranes were washed twice (20 min each time) in 1×SSC and 0.1% SDS at room temperature, followed by one wash (30 min) in 0.2×SSC and 0.1% SDS at 60° C. After washing membrane was exposed to X-ray film (Kodak, Rochester, N.Y.) overnight.

Western blot analysis. Cells were harvested in Eppendorf tubes and lysed for 15 min on ice in lysis buffer: 50 mM HEPES, 150 mM NaCl, 10 mM EDTA, 10 mM Na₄P₂O₇, 100 mM NaF, 2 mM sodium orthovanadate, pH 7.4, supplemented before use with 1% Triton X-100, 1 mM phenylmethylsulfonyl fluoride (PMSF), 1 μg/mL aprotinin, 2 mM leupeptin. Protein concentration was determined in the lysed cells' supernatants, and aliquots containing 50 μg of protein were diluted in SDS-PAGE sample buffer, boiled for 5 min, cooled, centrifuged and separated in 10% SDS-PAGE. Proteins were transferred onto PVDF Immobilon-P membrane (Millipore, Bedford, Mass.). Membrane was incubated with polyclonal rabbit antiserum against human PTTG polypeptide fragment (1:3,000 dilution) at 4° C. overnight, followed by incubation with peroxidase-linked secondary antibody (Santa Cruz Biotechnology, Santa Cruz. Calif.). Blots were visualized using ECL Western Blotting Detection Reagent (Amersham).

FACS analysis. To study cell cycle patterns cells were pelleted by centrifugation, washed in cold (4° C.) phosphate-buffered saline (PBS), resuspended in 1 mL of PBS, and then 2 mL of cold methanol was slowly added for cell fixation.

500 μL of propidium iodide (500 μg/mL PBS) together with 1 μL of ribonuclease A (10 units/μL) were added and cell suspension was vortexed. After incubation for 1 hour at 4° C., cell samples were analyzed by flow cytometry, performed on Becton-Dickinson FACScan. Analysis regions contained a minimum of 10,000 events. Cell cycle analysis was then performed on these data on Modfit software using Macintosh computer.

Cell labeling with triple mouse anti-CD3/CD19/CD45 flourochrome-labeled antibodies was performed using manufacturer protocol (Caltag Laboratories, Inc., Burlingame, Calif.). Shortly 15 μL of Caltag triple antibody was added to 100 μL of cell suspension, and then 180 μL of PBS was added and gently mixed. Tubes were incubated at room temperature for 15 min in the dark, cells were fixed using Cal-Lyse Lysing Solution (Caltag Laboratories, Inc.), washed with 3 mL of deionized water, pelleted by centrifugation and resuspended in 1 mL of cold PBS. Flow cytometry was done using a Becton-Dickinson FACScan.

All experiments were repeated at least 3 times, and the results of a typical experiment are presented immediately below.

PTTG expression was upregulated upon T-cell activation. It is known that T-cell activation needs a complex interaction between T lymphocyte and antigen-presenting cell. (Crabtree G R, Clipstone N A, Signal transmission between the plasma membrane and nucleus of Tlymphocytes, Annu Rev Biochem. 63:1045-1083 [1994]). T cell receptor interaction with major histocompatibility complex generates the most important signals for T-cell activation, although some adhesive interactions as well as costimulatory molecules are also of great significance here. T-cell surface molecules appear to provide costimulatory signals in two ways: either quantitatively by augmenting the second messenger generated by TCR/CD3 complex (for example, CD2), or qualitavely (for example, CD28) by generating intracellular biochemical signals distinct from those generated by TCR/CD3 complex. The procedure which we used for positive T-cell included T-cell binding to anti-CD2-coated immunomagnetic beads followed by cell culturing in anti-CD3-coated dishes. Although immunomagnetic beads themselves were not able to activate T-cells (no increase in PTTG mRNA expression and in cell cycling was observed) they could serve as a co-stimulator for T-cells. As an indirect argument for this suggestion could be our observation (data are not presented in this study) that T-cells isolated using cell sorter could not be activated neither with immobilized anti-CD3-antibodies, nor with PHA.

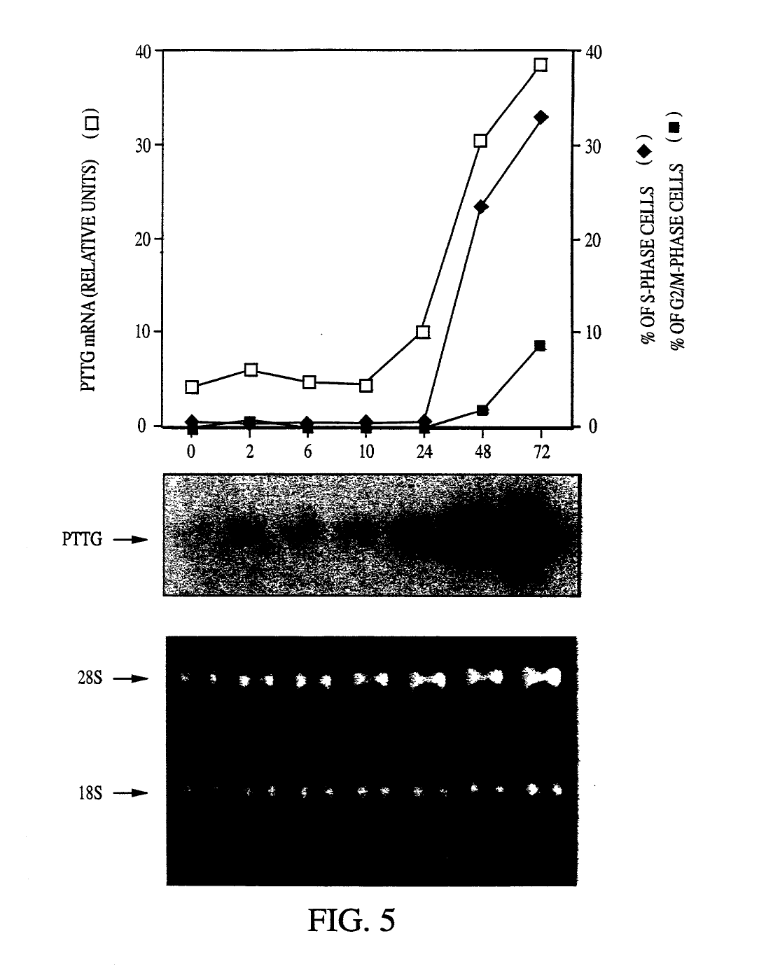

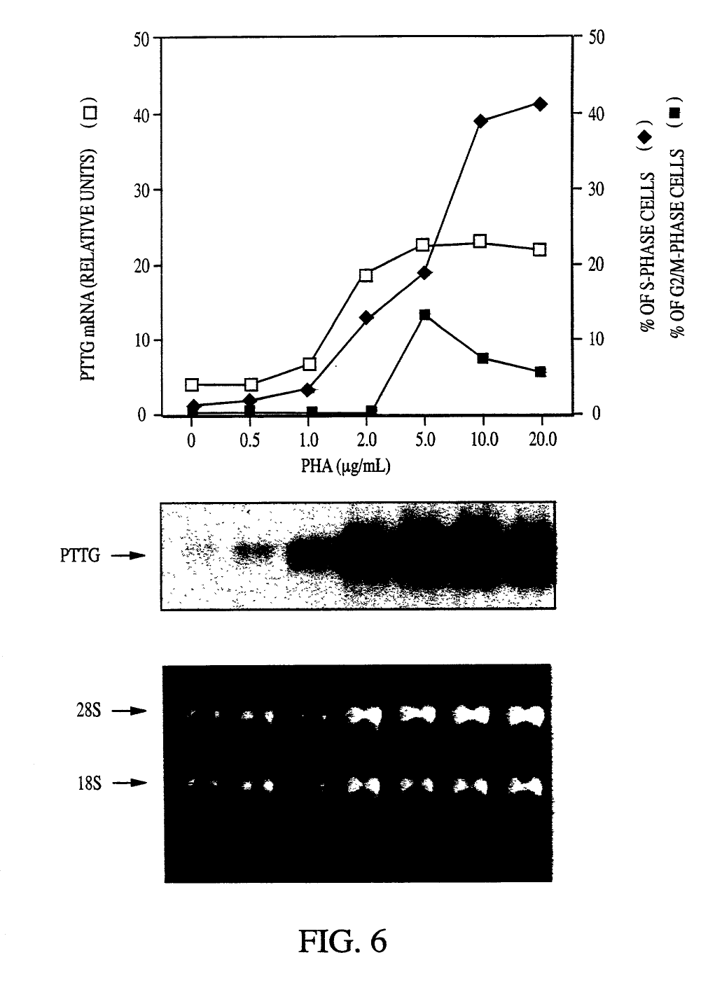

After resting human T cells were treated with a T-cell mitogen CD3 antibody, T-cells began to proliferate as shown by cells sequentially entering S phase and G2/M phase (FIG. 5). PTTG mRNA expression was practically non-existent in resting T-cells but it was dramatically increased as percentage of cells in S and G2/M phase became substantial. After resting T-lymphocyte cells were stimulated with another mitogen (phytohemagglutinin [PHA]), a similar time course was seen for both T-cell proliferation and PTTG expression (data not shown). T-cell proliferation and PTTG expression after 3 days of PHA stimulation were similarly dependent on PHA concentration up to 5 μg/mL (FIG. 6). Higher concentrations of PHA further increased S phase but decreased G2/M phase, but had no further effect on PTTG expression.

IL-2 is the most important early T cell activation gene. (Crabtree, G. R. and Clipstone, N. A., Signal transmission between the plasma membrane and nucleus of T lymphocytes, Annu Rev Biochem. 63:1045-1083 [1994]; Weiss A., T lymphocyte activation. In: Paul W E, ed. Fundamental Immunology. 3rd Edition. New York: Raven Press, Ltd. [1993] pp. 467-497; Kronke, M. et al., Cyclosporine A inhibits T-cell growth factor gene expression at the level of mRNA transcription, Proc Nat Acad Sci USA. 1984;81:5214-5218 [1984]).

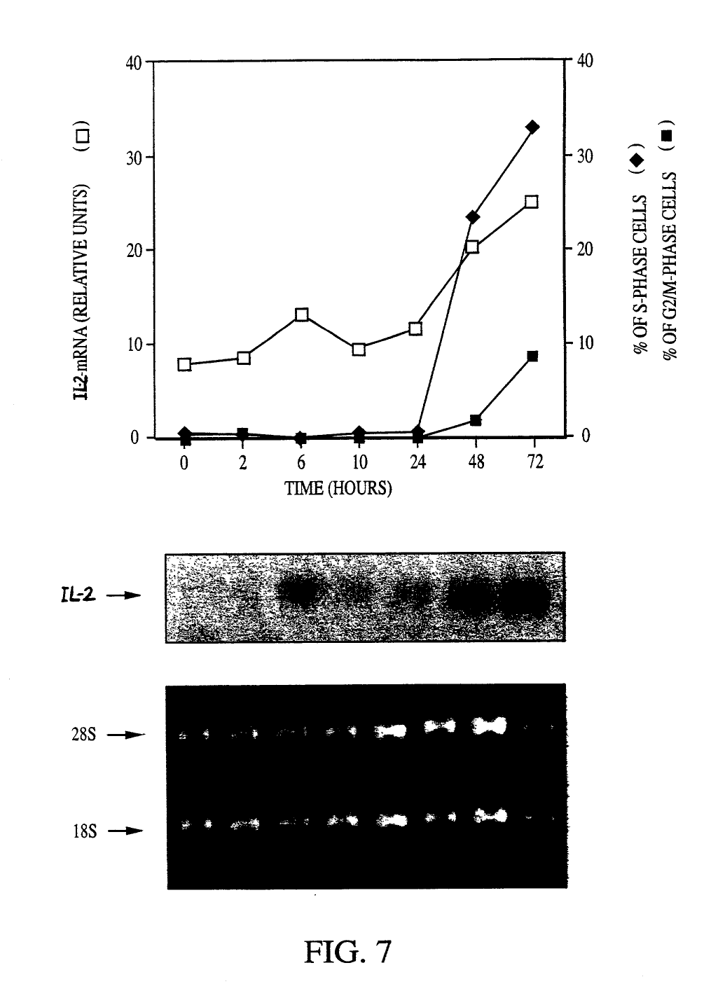

In contrast to the expression of PTTG, which was upregulated at later time, interleukin 2 (IL-2) expression was increased early and before cell proliferation (FIG. 7). IL-2 expression was first increased 6 hours after induction by immobilized anti-CD3 antibody application, long before any cells were in S or G2/M phase, suggesting this phase of IL-2 expression was independent of cell proliferation. At that time neither PTTG mRNA expression, nor significant amount of S-phase could be seen. Another phase of IL-2 elevated expression occurred 48 hours after the beginning of T cell activation, after T-cells had begun to proliferate. At that time considerable amount of PTTG mRNA and both S- and G2/M-phase cells were observed, which implies that IL-2 mRNA was already produced by proliferating T cells.

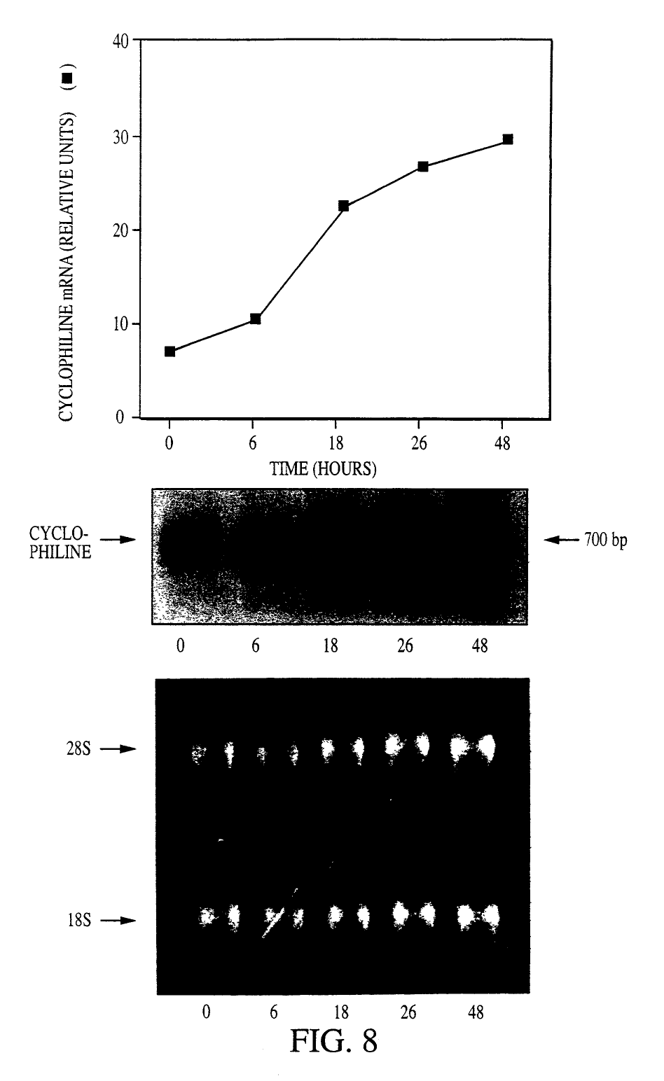

Cyclophilin, a house-keeping gene that also is an intracellular cyclosporin A-binding protein, was also increased as T-cells were activated by PHA (FIG. 8). It is known that cyclophilins serve as intracellular cyclosporin A-binding proteins. (Crabtree G R and Clipstone N A, Signal transmission between the plasma membrane and nucleus of T lymphocytes Annu Rev Biochem. 63:1045-1083 [1994]). Cyclophilins possess peptidyl prolyl-cis-trans isomerase activity involved in the catalysis of the cis-trans isomerization of proline residues in polypeptide substrates. This enzymatic activity may play some role in T-cell signal transduction cascade, perhaps by catalyzing the correct folding of an inactive signaling intermediate into an active form. However, strong arguments exist that this simplified model is not the main mechanism of cyclosporin A action. It was shown that cyclosporin A-cyclophilin complex can block the action of calcineurin (calcium/calmodulin-regulated serine/threonine protein phosphatase) via regulating nuclei entry of transcription factor NF-AT (nuclear factor-activated T cells) which in its turn regulates T cell specific IL-2 gene expression. (Crabtree GR and Clipstone NA, Signal transmission between the plasma membrane and nucleus of T lymphocytes. Annu Rev Biochem. 63:1045-1083 [1994]). We found that the level of cyclophilin mRNA expression is significantly increasing after PHA-induced T cell activation, which implies that the activated T-cells should be more sensitive to the action of cyclosporin A than the resting T-cells because they have higher cyclophilin levels.

It is known that both IL-2 (Crabtree and Clipstone [1994]) and PTTG (Kakar SS, Molecular cloning, genomic organization, and identification of the promoter for the human pituitary tumor transforming gene, Gene. 240:317-324 [1999]) gene promoter contain AP-1 binding regions which suggests the existence of common regulators of expression of these two genes. It is also accepted that a known immunosuppressor cyclosporin A exerts its inhibitory action towards T lymphocytes via IL-2 gene suppression and this leads to the inhibition of DNA synthesis in T cells. (Crabtree and Clipstone [1994]; Kronke M et al., Cyclosporine A inhibits T-cell growth factor gene expression at the level of mRNA transcription, Proc Nat Acad Sci USA. 81:5214-5218 [1984]).

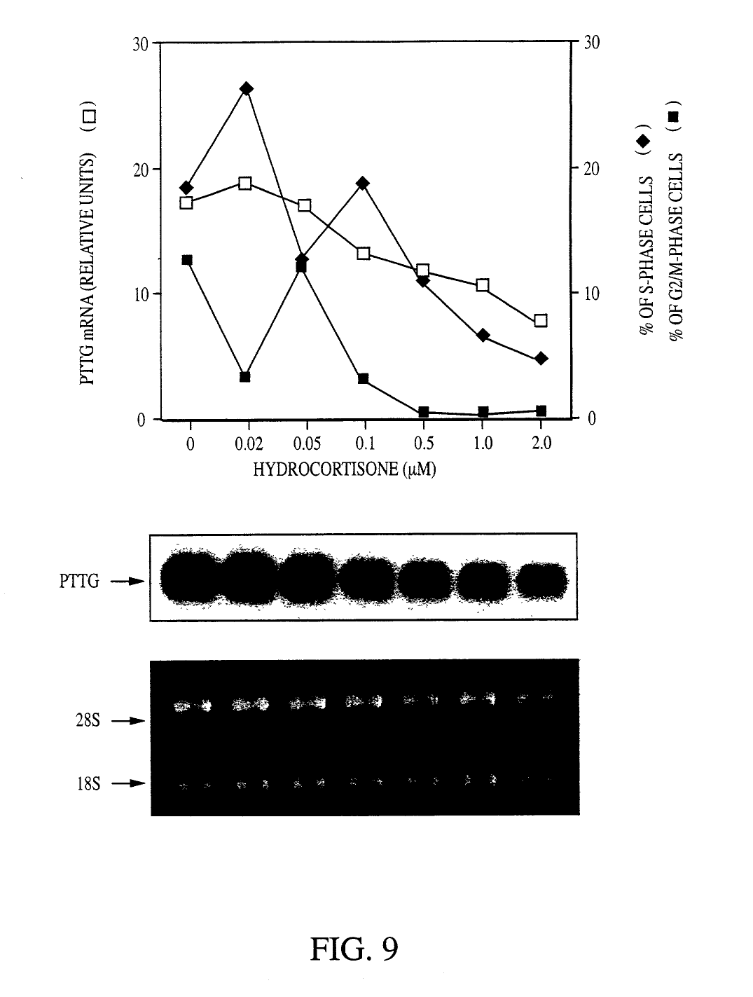

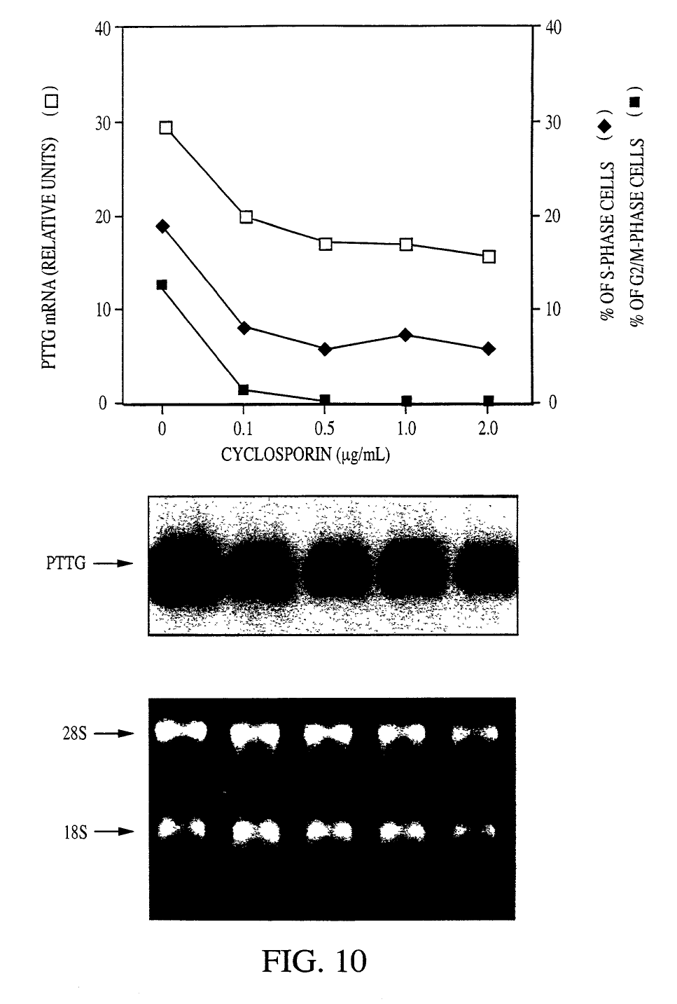

Although glucocorticoids have different (from cyclosporin A) intracellular receptors, they were also shown to suppress IL-2 and interferon-gamma production and to favor the production of IL-4 in T cells. (Clerici M, Trabattoni D, Piconi S et at. A possible role for the cortisol/anticortisols imbalance in the progression of human immune deficiency virus. Pssychoneuroendocrinology. 22 Suppl 1:S27-S31 [1997]). Another mechanism previously described for glucocorticoids' action is based on the induction of T cell apoptosis. (Cidlowski J A et al., The biochemistry and molecular biology of glucocorticoid-induced apoptosis in the immune system, Recent Prog Horm Res. 51:457-490 [1996]). We found dose-dependent inhibition by hydrocortisone of both PTTG mRNA expression and the amount of S- and G2/M-phase T cells, although in low doses (final concentration 20 nM) this steroid hormone slightly increased these indexes. Immunosuppressants hydrocortisone (FIG. 9) and cyclosporin A (FIG. 10) inhibited PHA-stimulated T cell proliferation and PTTG expression. The inhibitory effect of cyclosporin A was much stronger, while hydrocortisone action was even slightly stimulatory at low doses and was inhibitory at concentrations higher than 100 nM. These results further implied that PTTG expression is associated with T-cell proliferation. In general, even in the presence of very high hydrocortisone doses one can observe some PTTG expression and T cell cycling, while cyclosporin A suppresses T-cell functions in a much stronger manneer. It should be also noted that hydrocortisone did not inhibit PTTG expression in Jurkat leukemia T cell line (see below).

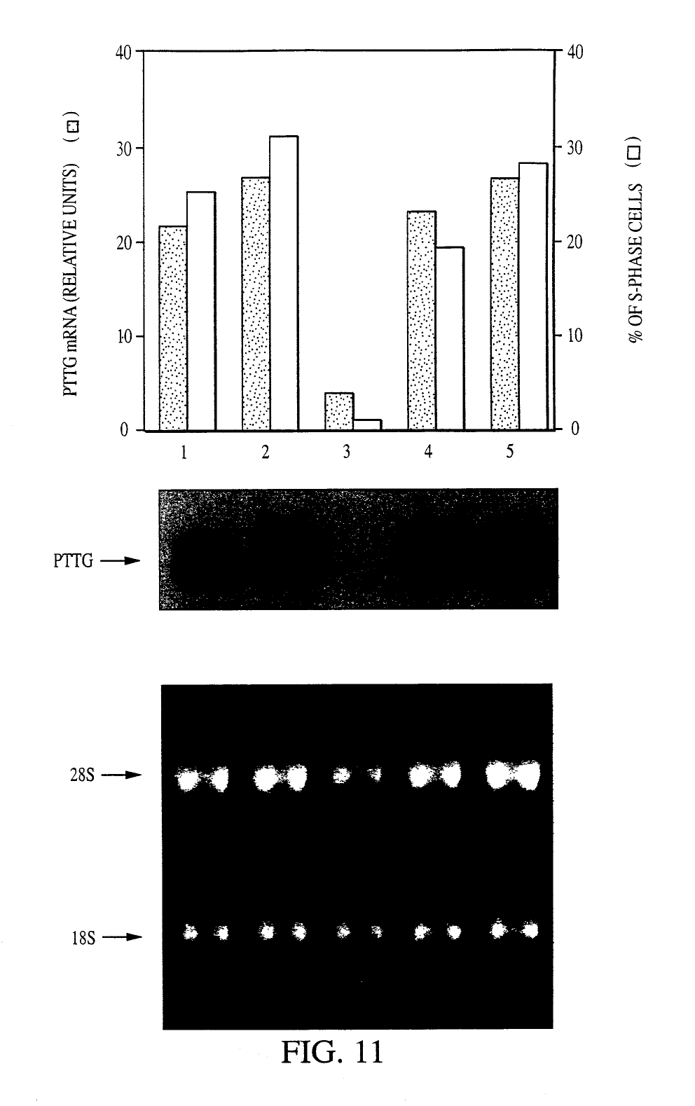

PTTG expression in leukemic cells. Human leukemia cell lines HL-60 and Jurkat grow in 10%-FBS-supplemented RPMI-1640 medium without CD3 or PHA as shown by the presence of S phase cells (FIG. 11). The abundance of PTTG mRNA in HL-60 and Jurkat cells was comparable to that in the activated T-cells by PHA or CD3 antibody. Among three immunomodulators used (cyclosporin A, hydrocortisone, and TGF-β1), only cyclosporin A acted in similar way on PTTG mRNA expression and cell cycling in both normal T-cells and Jurkat T cells, while the effects of hydrocortisone and TGF-β1 were specific for each of these two experimental models.

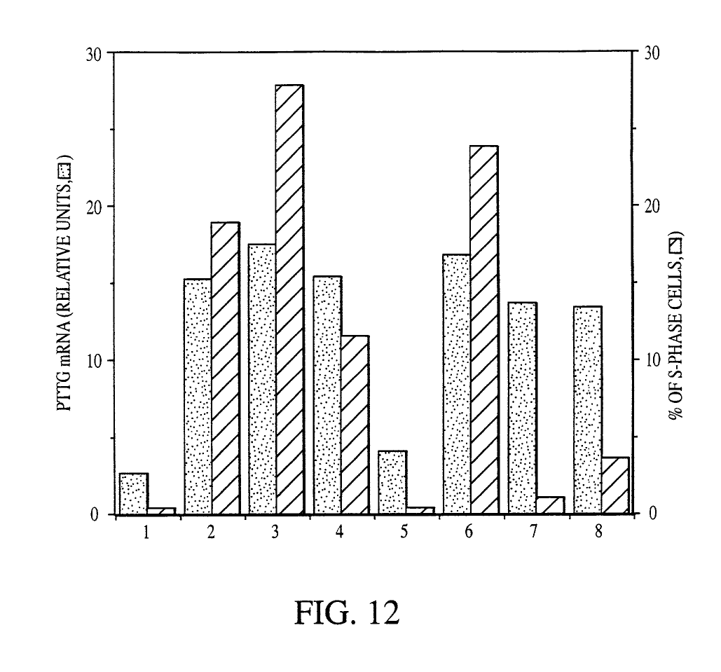

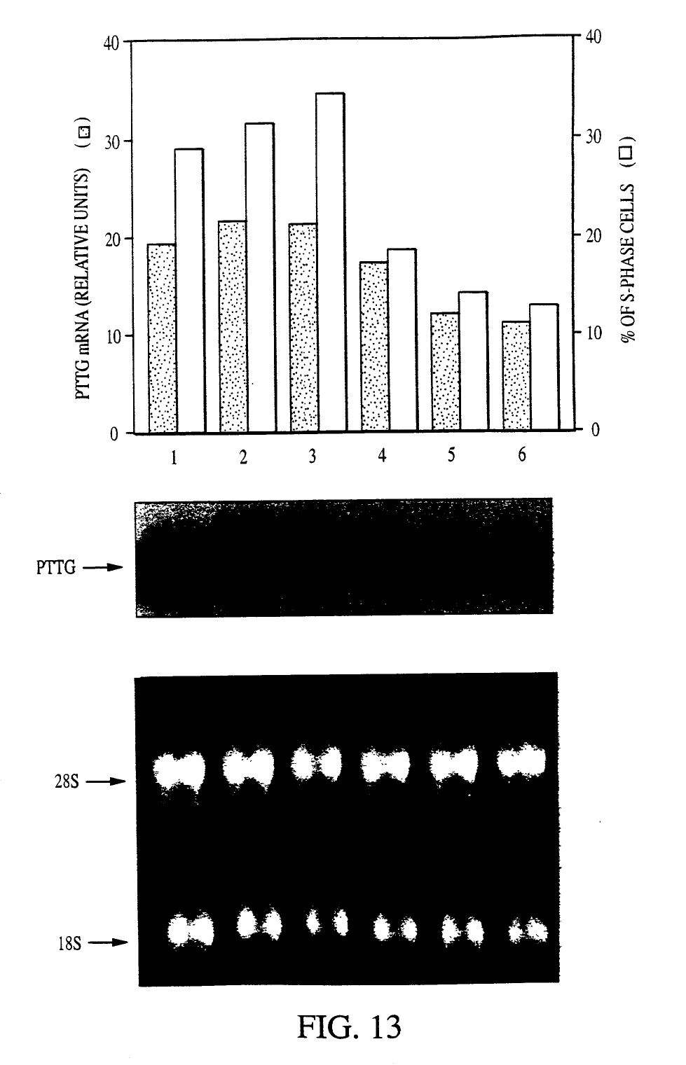

Cell cycle-dependent PTTG expression. We have shown that PTTG mRNA expression was cell-cycle dependent with peak expression at G2/M in a tumor cell line. We found a similar dependence of PTTG expression on cell cycle in T cells. This dependence was much more prominent in the case of normal T cells (FIG. 12) than in the case of Jurkat T-cell leukemia line (FIG. 13). Although the direction of revealed changes in the amount of S-phase cells and level of PTTG mRNA expression in normal T cells and Jurkat T cell line were mostly similar, the amplitude of these changes was found to be lower in the malignant Jurkat cells probably because of Jurkat cells' immortality and high basal levels of PTTG.

We found that CD3 antibody-induced activation of T-cells was inhibited to different extents by cyclosporin A, hydrocortisone, aphidicolin (S phase inhibitor), or nocodazole (G2/M phase blocker), while TGF-β1 (10 ng/nL final concn) had no significant effect neither on PTTG mRNA nor on the amount of S- or G2/M-phase cells (FIG. 12). At the same time, neither fresh 1% or 10% FBS nor a mixyure of phytohemagglutinin (PHA) and phorbol-12-meristate-13-acetate (PMA) [i.e., PHA+PMA mixture] considerably elevated PTTG level in Jurkat T-cell line and this mixture even decreased it. Jurkat cells were used for comparison and they were treated with PHA+PMA mixture which is used for its mitogenic action for Jurkat T cells after being cultured for 48 hours in 1% FBS-supplemented medium. Changes in the amount of S-phase cells were also parallel to changes in the level ofPTTG mRNA expression. Cyclosporin A and TGF-β1 decreased both PTTG mRNA level and the amount of S-phase Jurkat cells, while hydrocortisone did not change these indexes even used in 10 μM final concentration (data are not shown).

It should be noted that TGF-β1 is well known for its bi-directional action on cell functions including cell proliferation which strongly depends on the properties of cell targets and the conditions of their treatment. At the same time when Jurkat T cells were used we detected TGF-β1-triggered inhibition of both PTTG mRNA expression and cell cycling. Although this effect was not observed in the presence of fresh FBS added to culture medium (data are not shown) and could be seen only after Jurkat cells' treatment with PHA+PMA mixture.

These results show that the expression of PTTG can be easily regulated in normal T-cells. It is induced to high level by both non-specific (PHA) and specific (anti-CD3 antibodies) T-cell activators, and this induction can be inhibited by known immunosuppressors—cyclosporin A and hydrocortisone. Importantly, preliminary data showed that the transfection of PHA-activated T cells with DNA encoding PTTG C-terminal region caused a decrease in the amount of S-phase cells and their accumulation in G2/M-phase of cell cycle.

Interestingly, hydrocortisone did not inhibit PTTG expression in Jurkat T cell line widely used for studying of T-cell functions, while another immunomodulator, TGF-β1 did not affect PTTG expression in normal T-cells, but decreased it in Jurkat cells. Thus, the results of study of PTTG functioning in Jurkat T cell line cannot be directly applied for normal T-cells, probably because of high basic level of expression of PTTG oncogene in the malignant cells.

Normal T cells and Jurkat T-cell line also possessed different sensitivity to the action of cell cycle inhibitors (aphidicolin and nocodazole). Aphidicolin stopped Jurkat cells in S-phase, decreasing the amount of G2/M-phase, while no significant changes in PTTG mRNA level were observed (data not shown). Nocodazole stopped these cells in G2/M-phase and S-phase cells were poorly detected in its presence, while some increase in PTTG mRNA expression was observed after nocodazole treatment of Jurkat cells. Probably, normal T-cells are more sensitive to the toxic effect of cell cycle inhibitors and in their presence both PTTG mRNA level and the amount of cycling cells were decreased.

The results presented herein show that targeting PTTG and PTTG expression is useful both for anti-neoplastic therapy and also for immunosuppressive therapy.

While the present invention is not committed to or dependent any particular mechanism of action, PTTG mRNA induction and parallel S-phase increase during normal T-cell activation imply that the mechanism of PTTG cell transforming action could be in its overexpression and resulting increase in cell cycling rather than in its misregulating effect on chromatide separation and resulting aneuploidy.

Example 18: PTTG1 Expression in Breast and Ovarian Tumor Tissues

Patients and Tissues. Separate samples of breast (n=13) and ovarian (n=15) tumors were obtained from consecutive unselected patients after surgical resection and either immersed in liquid nitrogen and stored at −70° C., or fixed in 10% formalin for analysis. Normal breast (n=14) and ovarian (n=9) postmortem tissues were obtained from the Brain & Tissue Banks for Developmental Disorders at The University of Maryland, Baltimore (mean±SEM age; 44±7.2 yr.). Non-degraded RNA was obtained from 25 of the 28 tumor tissues and 20 of the 23 normal tissues and used for further analysis. Twelve cases of breast carcinoma (mean±SEM age; 60±3.5 yr.) and thirteen cases of ovarian cancer (mean±SEM age; 56±4.9 yr.) (Table 21) were studied. Histologic evaluation was independently recorded by a pathologist.

Cell Cultures. MCF-7 (breast cancer), MDA-MB231 (estrogen receptor [ER]-negative breast cancer cells) and SKOV-3 (ovarian cancer) cells obtained from ATCC were maintained in phenol red-free DMEM with 10% FCS, pretreated with dextran-coated charcoal (CSS) for 3 days, prior to treatment with diethylstilbestrol (10⁻⁸ M and 10⁻¹⁰ M), and/or ICI-182780 (Tocris) (10⁻⁷ M and 10⁻⁸ M) for 48 h as previously described (Heaney, A. P. et al. [1999]).

Northern blot analysis. Total RNA was extracted from cell cultures (˜3×10⁷ cells) and excised tissues with TRIzol. RNA derived from JEG-3 choriocarcinoma cells served as a positive control for PTTGI expression. Electrophoresed RNA was transferred to Hybond-N nylon membranes (Amersham International, Buckinghamnshire, UK), and hybridized at 68° C. with human PTTG cDNA as previously described (Heaney, A. P. et al. [1999]). PTTG1 mRNA expression was normalized to β-actin expression and expressed as fold-increase relative to either matched normal mucosa from the same individual (8 breast cases), or the mean PTTG/ actin ratio measured in normal breast (n=13) and ovarian tissue (n=7)

Western blot analysis. Proteins were prepared from breast and ovarian tissues using RIPA buffer (Heaney, A. P. et al [1999]), denatured in loading buffer, and soluble proteins (50 μg by Bradford assay) were separated by electrophoresis (12% SDS-PAGE), transferred to PVDF membranes (Amersham), incubated in 5% non-fat milk in PBS-0.05% Tween solution, followed by incubation with antibodies to PTTG (1:5000), and β-actin (1:2500; Sigma). Blots were washed, incubated with appropriate horse radish peroxidase- conjugated anti-IgGs, washed and complexes were then visualized by ECL chemiluminescence detection.

Differences were assessed by ANOVA or the unpaired t-test when appropriate.

### Results

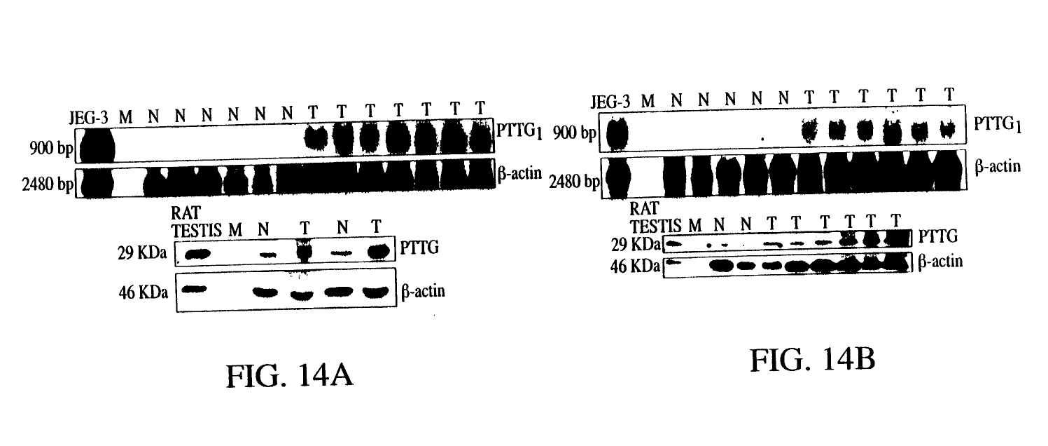

PTTG1 is overexpressed in breast and ovarian tumors. Increased PTTG1 mRNA expression was observed in 12 of 12 breast and 12 of 13 ovarian carcinomas (breast carcinoma exhibited 2.5±0.3 fold increase; ovarian carcinoma exhibited 3.5±0.6 fold increase) in comparison to normal breast or ovarian tissue respectively (Table 21; FIG. 14). Highest PTTG mRNA expression was detected in breast and ovarian tumors which invaded surrounding lymphatic or vascular structures (breast tumor exhibited 3.5±0.45 fold increase; ovarian tumor exhibited 3.8±0.7 fold increase) compared to tumors confined to the breast or ovary (breast tumor exhibited 1.9±0.35 fold increase; ovarian tumor exhibited 3.2±1.0 fold increase), although this difference was only statistically significant in the breast tumors (p=0.03).

TABLE 21
PTTGI expression in breast and ovarian carcinomas.

| | | | Lymph node | PTTGexpression |
|---|---|---|---|---|
| No. Patient Age | | Diagnosis | invasion | (fold-increase) |
| Breast Tumors: | | | | |
| 1 | 49 | CAI^{a} | NA | 2.7 |
| 2 | 58 | CAI | −ve | 1.3 |
| 3 | 83 | infiltrating ductal Ca^{b} | −ve | 2.3 |
| 4 | 42 | invasive ductal Ca | −ve | 1.0 |
| 5 | 80 | infiltrating ductal Ca | −ve | 1.3 |
| 6 | 56 | invasive Ca (mucinous) | −ve | 2.0 |
| 7 | 53 | invasive Ca (tubular) | −ve | 1.2 |
| 8 | 67 | infiltrating ductal Ca | −ve | 3.9 |
| 9 | 56 | invasive duct cell Ca | −ve^{c} | 3.8 |
| 10 | 65 | invasive ductal Ca | +ve | 4.1 |
| 11 | 52 | infiltrating ductal Ca | +ve | 2.2 |
| 12 | 62 | infiltrating ductal Ca | +ve | 4.0 |
| | | | | 2.5 ± 0.3 |
| | | | | (Mean ± SEM) |
| Ovarian Tumors: | | | | |
| 13 | 18 | Sertoli Leydig cell tumor | −ve | 1.9 |
| 14 | 45 | endometroid Ca | −ve | 0.9 |
| 15 | 62 | endometroid Ca^{d} | −ve | 2.2 |
| 16 | 57 | Serous Cystadenoma | −ve | 4.9 |
| 17 | 62 | Mullerian Papillary | −ve | 6.3 |
| | | Adeno Ca (HG)^{e} | | |
| 18 | 50 | Mucinous Cystadeno Ca | +ve | 1.1 |
| 19 | 47 | Cystadenocarcinoma | +ve | 1.8 |
| | | (HG)^{e} | | |
| 20 | 67 | Serous Papillary Ca (HG) | +ve | 3.3 |
| 21 | 67 | Serous Papillary Ca (HG) | +ve | 3.3 |
| 22 | 82 | Adeno Ca (HG) | +ve | 3.8 |
| 23 | 48 | Mullerian Serous | +ve | 4.2 |
| | | Papillary Ca | | |
| 24 | 51 | Mullerian Adeno | +ve | 5.1 |
| | | Ca (HG) | | |
| 25 | 78 | Mullerian Serous | +ve | 7.6 |
| | | papillary Adeno | | |
| | | Ca (HG) | | |
| | | | | 3.5 ± 0.6 |
| | | | | (Mean ± SEM) |

^{b}Ca = carcinoma
^{c}Lymphovascular invasion present
^{d}bilaterla involvement
^{e}HG = high grade

Estrogen-regulation of PTTG and its overexpression in breast and ovarian tumors from the mostly post-menopausal women in this study may seemingly appear a paradox. However, estrogens are an important growth factor in both pre- and post-menopausal women and the prevalence of hormone-dependent breast cancer increases with age, as does the incidence of breast cancer. After menopause, peripheral tissues produce sufficient estradiol concentrations to stimulate tumor growth, and about 80% of postmenopausal women with ER-positive breast tumors respond to antiestrogen-treatment, and adjuvant tamoxifen is standard therapy for postmenopausal women (Weidner, N. et al., Tumor angiogenesis: a new significant and independent prognostic factor in early-stage breast carcinoma, J. Natl. Cancer Inst. 1992; 84:1875-87 [1992]).

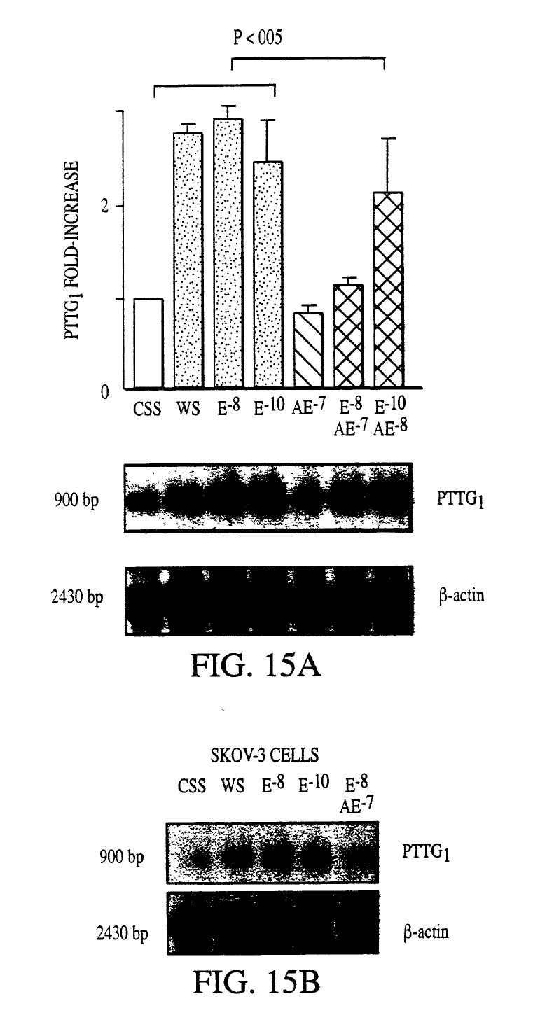

Estrogen regulates PTTG1 expression in breast and ovarian cancer cells in vitro and correlates with tumor estrogen receptor expression. Estrogen (diethylstilbestrol 10⁻⁸ M to 10⁻¹⁰ M) induced about a 2-9 fold-increase in PTTG1 mRNA expression in MCF-7 breast cancer cells (FIG. 15a; p<0.01), and in ovarian cancer cells (FIG. 15b, inset). Estrogen-mediated induction of PTTG1 was partially abrogated or blocked by co-incubation with the antiestrogen ICI-182780 (10⁻⁷ M to 10⁻⁸ M) (FIG. 15, p<0.01). PTTG1 mRNA expression was unaltered following treatment of estrogen receptor (ER)-negative MDA-MB231 breast cancer cells with estradiol (data not shown), confirming the requirement of the ER for estrogen-mediated PTTG1 mRNA induction.

PTTG regulates bFGF secretion (Pei, L and Melmed, S., Isolation and characterisation of a pituitary tumor-transforming gene (PTTG), Mol. Endocrinol. 11:433-441 [1997]) and in turn bFGF regulates PTTG in NIH 3T3 fibroblasts. Therefore, the incomplete suppression of estrogen-induced PTTG by ICI-182780 observed here may be due to medium-derived growth factors whose action may not be altered by antiestrogens. Breast and ovarian cancers are epithelial in origin and as stromal/epithelial components of normal and tumor tissue may differ, care must be exercised in interpretation. As stroma is poor in cellularity, and contributes little to the total DNA or RNA content in specimens (Orr-Weaver, T. L., The difficulty in separating sisters, Science. 285:344-345 [1999]), the detection of abundant PTTG expression in breast and ovarian tumors in comparison to expression in normal breast tissue, using Northern and Western blot analysis appears appropriate and is validated by other studies (Orr-Weaver [1999]).

### Example 19: PTTG-C Hypersensitizes Cancer Cells to Treatment with Cytotoxic Anti-cancer Drugs

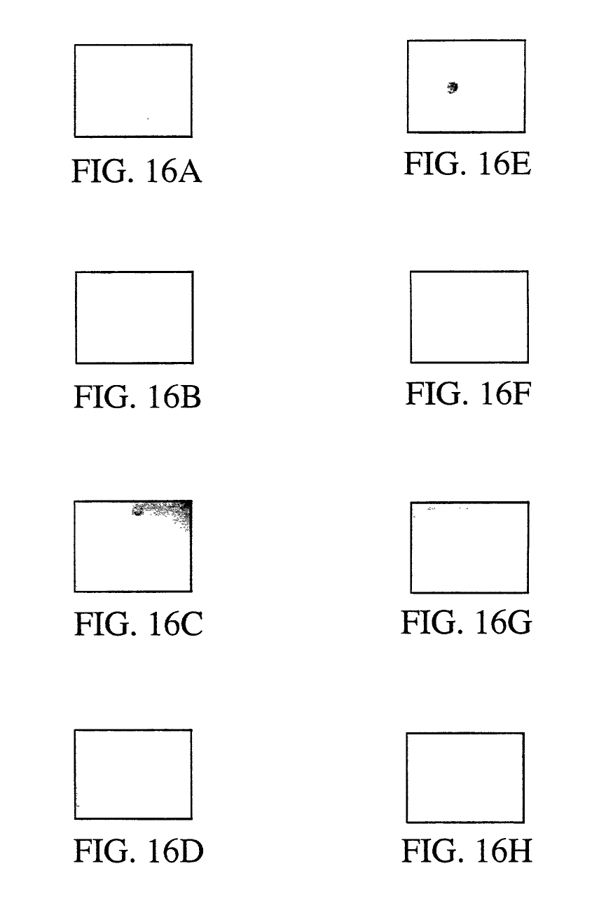

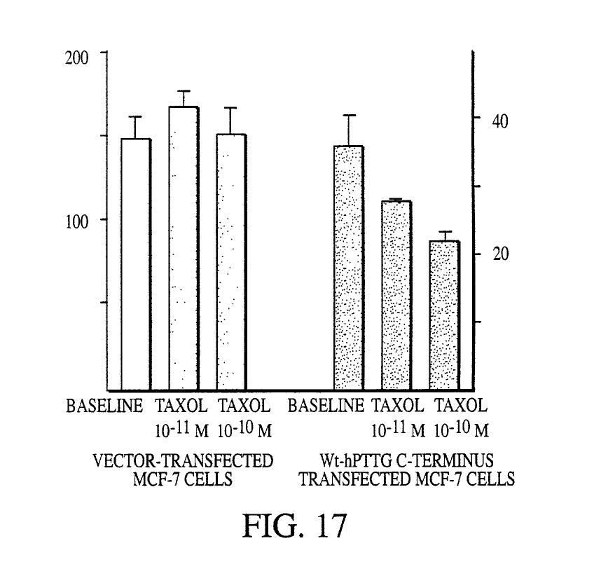

Co-expression of PTTG-C (wtPTTG C-terminus) in cancer cells, disrupts PTTG-mediated signaling, prevents colony formation in soft agar and in vivo tumor formation in nude mice. (See, Example 16 hereinabove). Furthermore, expression of PTTG-C in cancer cells, such as but not limited to breast and ovarian cancer cells, hypersensitizes the cells to treatment with cytotoxic anti-cancer drugs, such as paclitaxel (Taxol). For example, FIGS. 16 and 17 show that treatment of wtPTTG C-terminus-transfected MCF-7 (stable transfection) breast cancer cells with paclitaxel inhibited colony formation at lower doses (10⁻¹¹ M to 10⁻¹⁰ M) than those necessary to inhibit colony formation in the control vector-transfected cells (10⁻⁹ M) (FIGS. 16 and 17).

### Example 20: Modulation of Angiogensis by PTTG

Materials. Rat tail collagen type I was obtained from Sigma Chemical Co. (St. Louis, Mo.), and the modified Boyden chamber, and Transwells®, from Corning Costar (Cambridge, Mass.). Human recombinant, anti-bFGF antibody, and pre-immune goat IgG were purchased from R&D systems (Minneapolis, Minn.), growth factor reduced Matrigel basement membrane matrix (GFR Matrigel) from Becton Dickinson (Bedfold, Mass.), and fertilized White Leghorn chicken eggs from Chino Valley Ranchers (Arcadia, Calif.). All standard chemicals used were of the highest available commercial grade.

Cell culture. NIH-3T3 cells were cultured in low glucose DMEM (GIBCO-BRL) supplemented with 10% bovine calf serum (BCS) and antibiotics. HUVECs (Clonetics San Diego, Calif.), were grown in EGM medium according to the vendor's instructions, and were grown to less than ten passages for all experiments.

Stably transfected NIH-3T3 cells (2×10⁶) expressing wild type, mutant hPTTG, or vector alone, as previously described (Zhang, X. et al., Structure, expression, andfunction of human pituitary tumor-transforming gene (PTTG), Mol Endocrinol. 13:156-166 [1999]), were plated in 100-mm gelatinized dishes. Western blot analysis confirmed that equivalent amounts of PTTG protein were expressed in both wild type-PTTG and mutant-PTTG transfected cells. After 24 hours, the maintenance medium was replaced with 10 mL serum-free DMEM and the cells incubated a further 48 hours. This conditioned medium (CM) was then harvested from wild type hPTTG (WT-hPTTG-CM), mutant hPTTG (M-hPTTG-CM), and vector alone (C-CM) transfected NIH-3T3 cells and from non-transfected NIH-3T3 cells (N-CM), each CM type being filtered separately through a sterile 0.2 μm pore filter to remove debris and then being stored until further study.

bFGF ELISA. Conditioned medium (1 mL) was lyophilized with SpeedVac (Savant, Farmingdale, N.Y.), resuspended in 100 mL phosphate-buffered saline (PBS), and bFGF concentration was assayed (Quantikine HS Human FGF basic Immunoassay Kit, R&D).

Endothelial cell proliferation assay. HUVECs were plated onto 48-well gelatinized culture plates at about 5000 cells/well for 24 hours. Medium was then replaced with equal aliquots of CM derived from cultures of transfected or non-transfected NIH-3T3 cells as described previously. As a positive control, DMEM was enriched with 1 ng/mL of recombinant human bFGF, and as a negative control, serum-free DMEM was used. To investigate activity of bFGF in each CM, 100 ng/ml anti-bFGF antibody or pre-immune goat IgG was first added to each CM. After 48 hours, HUVEC cells were trypsinized and counted with a Coulter Counter (Coulter Electronics, Hialeah, Fla.). All experiments were performed in triplicate.

Wound migration assay. The wound assay was performed as previously described with some modifications (Sato, Y and Rifkin, D B., Autocrine activities of basic fibroblast growth factor: regulation of endothelial cell movement, plasminogen activator synthesis, and DNA synthesis, J Cell Biol. 107:1199-1205 [1988]). Confluent monolayers of HUVECs in 35-mm gelatinized culture dishes were wounded by pressing a razor blade to cut the cell sheets and mark the plate. The blade was gently moved to one side to remove part of the sheet. Cells were then washed twice with PBS. The transfected-3T3 cell-derived CM described above was applied and the HUVECs incubated in CM for a further 16 hours. Cells were then fixed with absolute methanol, stained with Giemsa and photographed. Migration was quantified by counting cells in 100 μM×2500 μM sections from the cut edge under microscopy with an ocular grid. The values represent the mean derived from 3 random fields. All experiments were repeated in triplicate.

Modified Boyden chamber migration assay. Migration was also measured with 6.5-mm, 8.0 μm Transwells® as previously described with some modifications (Leavesley, D I et al., Integrin beta 1- and beta 3-mediated endothelial cell migration is triggered through distinct signaling mechanisms, J Cell Biol. 121:163-170 [1993]). The polycarbonate membrane was coated with 0.1 % gelatin (1 h at 37° C.). 600 μL of each CM sample was added to the lower chamber and incubated at 37° C. for 30 min. Sub-confluent HUVECs which had been cultured in the growth factor-free medium for 16 h were harvested, washed, resuspended in serum-free DMEM (100 μL) and added to the upper chamber. After 24-h incubation, all non-migrant cells were removed from the upper face of the membrane with a cotton swab and migrant cells on the lower face were fixed with absolute methanol, stained with Giemsa and photographed. For quantitative analysis, stained cells were subsequently extracted with 10% acetic acid, and absorbance determined at 595 nm.

Tube forming assay. Assay of capillary tube-like structure formation of HUVEC was performed with commercial GFR Matrigel. 24-well plates were thickly coated with 300 μL GFR Matrigel (11 mg/mL) and incubated at 37° C. for 30 min to promote gelling. HUVECs suspended in 500-μL aliquots of sample CM were added to each well to bring the final culture to about 5×10⁴ cells per well. After 24-h incubation, tube-formation was evaluated by phase-contrast microscopy and photographed by spot color digital camera (W. Nuhsbaum, Inc., McHenry, Ill.). Digital images were skeletonized with NIH-image software, and pixel numbers counted as previously described (Wojta, J., et al., Hepatocyte growth factor increases expression of vascular endothelial growth factor and plasminogen activator inhibitor-1 in human keratinocytes and the vascular endothelial growth factor receptor flk-1 in human endothelial cells, Lab Invest. 79:427-438 [1999]). All experiments were repeated in triplicate.

Chrorio-Allantoic Membrane (CAM) assay. To investigate angiogenic activity of each CM in vivo, fertilized White Leghorn chicken eggs were incubated at 37° C. without CO₂ in a humidified incubator (Brooks, PC, et al., Use of the 10-day-old chick embryo model for studying angiogenesis, Methods Mol Biol. 129:257-269 [1999]). After 3-day incubation, a round window was opened in the shell and 3 mL albumin were removed to detach the CAM from the shell. 10 mL of CM from WT-hPTTG, M-hPTTG, vector transfected and untransfected 3T3 cells were lyophilized separately with (SpeedVac) and each was resuspended in 100 μL PBS. 1 μg of bFGF in 5 μL PBS was used as a positive control and similarly concentrated serum-free DMEM was used as a negative control. On day 9, 5 μL of either concentrated CM, or the positive or negative controls were applied to 0.5 mg rat tail collagen type I sponge. Sample-soaked sponges were then placed onto the CAM. On day 13, shell windows were carefully extended and the sponge and surrounding CAM area photographed. For quantitative analysis, the number of blood vessels entering the collagen sponges was counted under stereomicroscopy at 25× magnification. Three eggs were used for each sample and experiments were repeated in triplicate.

Statistical analysis. Statistical analyses were performed using the Student's t test. All P values were two-tailed, and those less than 0.05 were considered significant.

### Results

bFGF concentration in conditioned media.

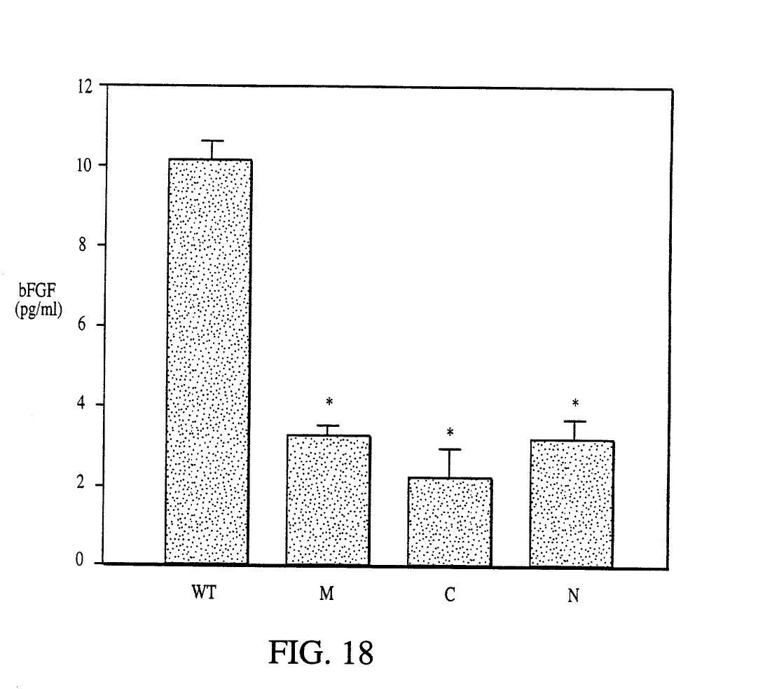

As hPTTG regulates bFGF secretion (Zhang, X. et al., Structure, expression, and function of human pituitary tumor-transforming gene (PTTG), Mol Endocrinol. 13:156-166 [1999]), we measured bFGF concentration in conditioned medium derived from stably transfected cells (about 2×10⁶) after 48-hour culture in serum-free medium (FIG. 18). The bFGF concentration in CM harvested from WT-bPTTG transfectants, was 10.5±0.56 pg/mL, markedly higher than bFGF levels in CM derived from other transfected cell lines ([pg/mL] Mut-hPTTG, 3.3±0.27; Ctr-vector, 2.3±0.72; normal 3T3 cells 3.3±0.56; p<0.01). The bFGF concentration in CM from mut-hPTTG, Ctr-vector- transfected and normal 3T3 cells did not differ. Total cell number and protein concentration at the time of CM collection were similar for each independent cell line or transfectant.

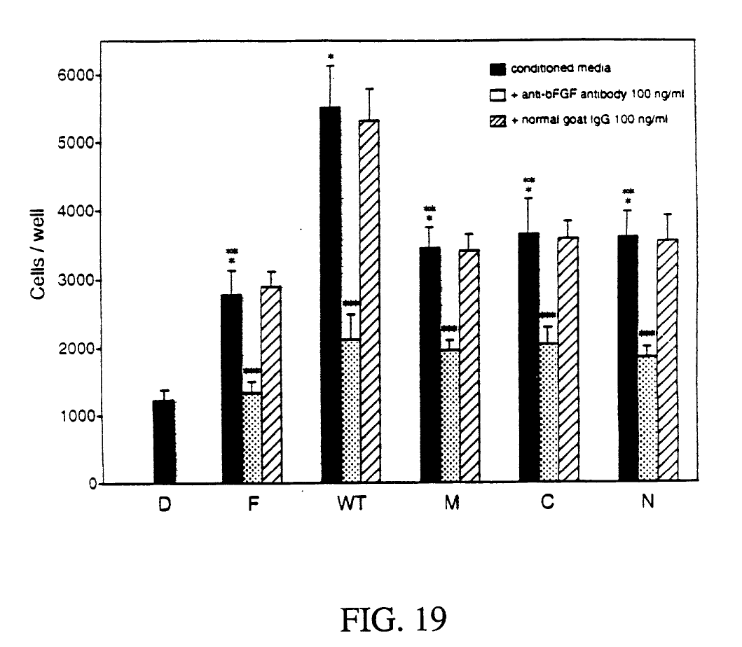

Endothelial cell proliferation assay. HUVECs were cultured in each CM for 48 hours after which the cell number was determined (FIG. 19). As expected CM derived from all cell lines exhibited proliferative activity in comparison with serum-free DMEM. CM from WT-hPTTG-transfected cells induced significantly higher cell proliferation than CM derived from Mut-hPTTG, Vector-transfected and normal 3T3 cells (p<0.01). Addition of anti-bFGF antibody to each CM suppressed proliferation activity by 62%, WT-hPTTG-CM; 43%, M-hPTTG-CM; 44%, C-CM; and by 49%, N-CM. However, cell proliferation after adding goat anti-bFGF antibody was still higher than in serum-free DMEM alone. Proliferation of HUVECs was not altered by adding pre-immune goat IgG to each CM, confirming that the induced proliferation was mediated by bFGF.

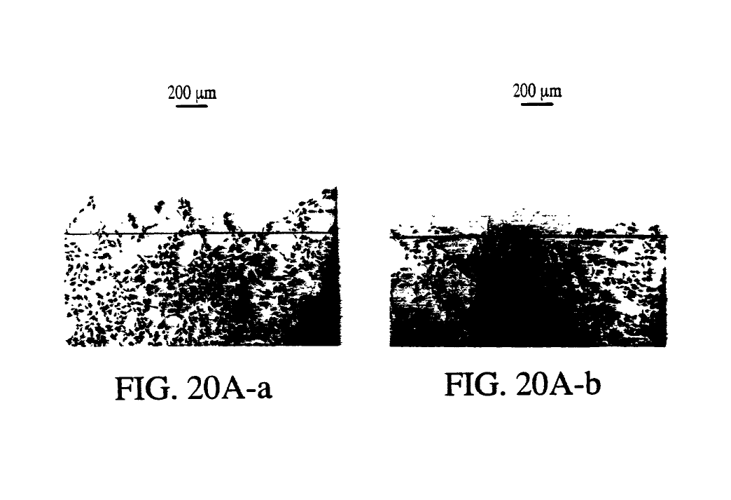

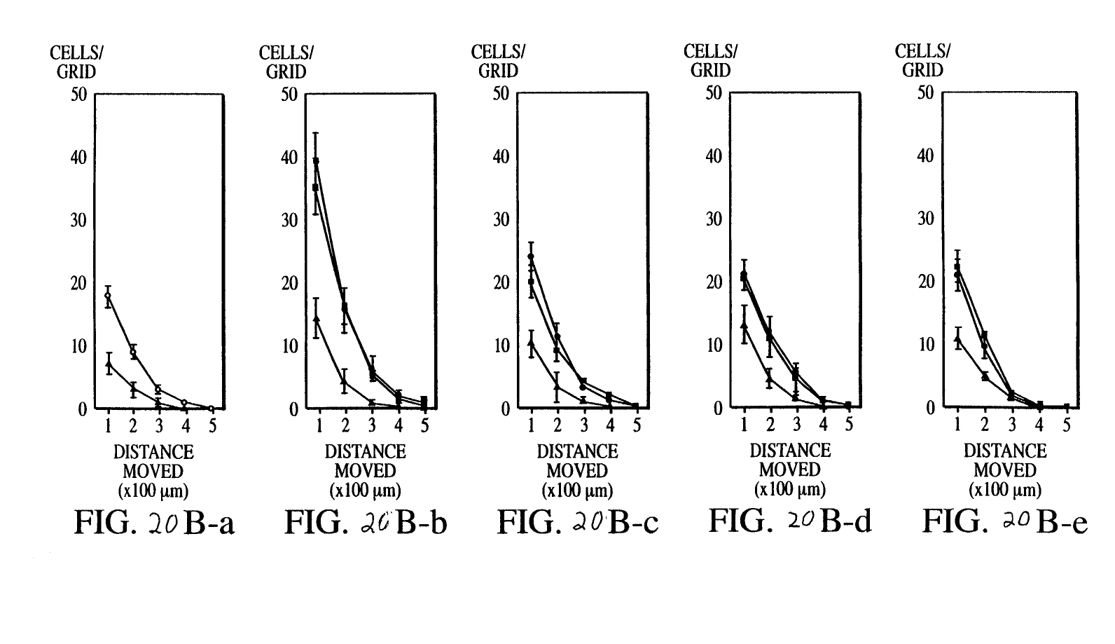

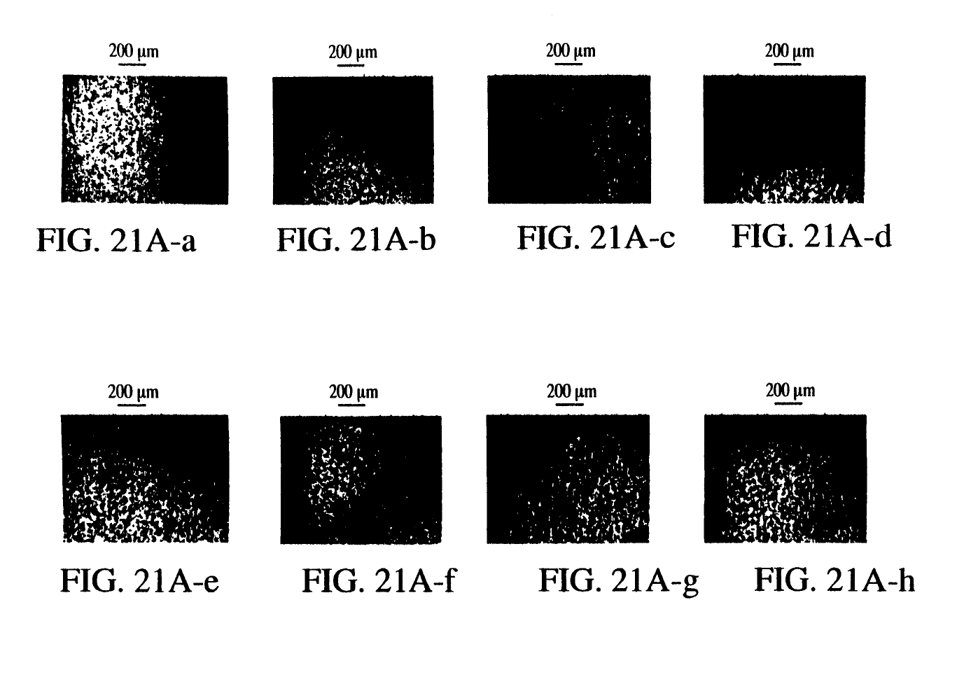

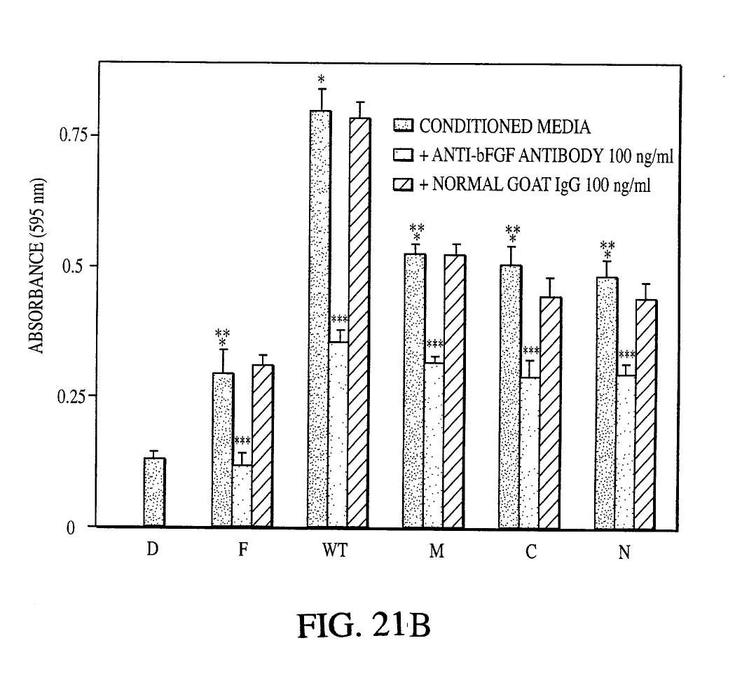

Endothelial migration in wound assay and Boyden chamber assay. Endothelial cell proliferation and migration were tested by using the wound healing assay and modified Boyden chamber assay In the wound assay (FIG. 20), migration was quantified by counting the number of HUVECs which migrated into the non-wounded region with a grid marked in 100-μm increments. HUVECs that had been incubated (48 h) in WT-hPTTG-CM migrated farther and in greater numbers than HUVECs that had been incubated in CM from the other cell lines, harboring Mut-PTTG, vector alone or untransfected 3T3 cells. Goat anti-bFGF antibody suppressed activity in all cell lines, but pre-immune goat IgG had no effect. Using the modified Boyden chamber assay conditioned medium from Wt-hPTTG transfectants induced HUVEC cell migration through membrane pores (FIG. 21; p<0.01). Similar results were obtained when transfected or non-transfected NIH-3T3 cells were plated in the lower chambers and HUVECs plated in the upper chamber in a co-culture manner, (data not shown). Anti-bFGF antibody suppressed migration activity in all cell lines similarly to what was observed in the wound assay. Suppressive effects by anti-bFGF antibody of WT-hPTTG-CM, M-hPTTG-CM, C-CM and N-CM were, in the same order, 55%, 40%, 43% and 39%. Inhibitory effects of the anti-bFGF antibody on CM mediated angiogenesis were more evident in WT-hPTTG-CM than in CM derived from the other cell lines. Thus, angiogenic activity of WT-hPTTG-CM is abrogated by neutralizing bFGF antibody. Addition of neutralizing bFGF antibody did not completely reverse the angiogenic effects of CM from wtPTTG-transfected cells, implying that some PTTG-directed angiogenesis is probably due to other CM-derived factors.

Similar angiogenic activity was observed using M-hPTTG-CM to C-CM and N-CM, and these were all significantly lower than angiogenic activity mediated by Wt-hPTTG-CM, demonstrating that WT-hPTTG-CM induces strong angiogenic activity. These results also imply that the proline-rich domains of the PTTG carboxy terminal end of PTTG peptide are important, not only with respect to transforming activity, but also for PTTG-mediated angiogenic properties.

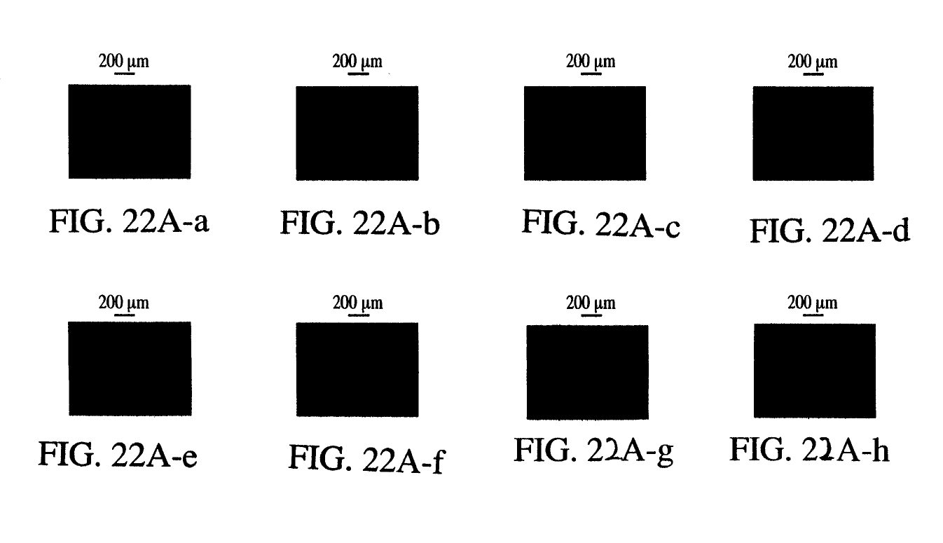

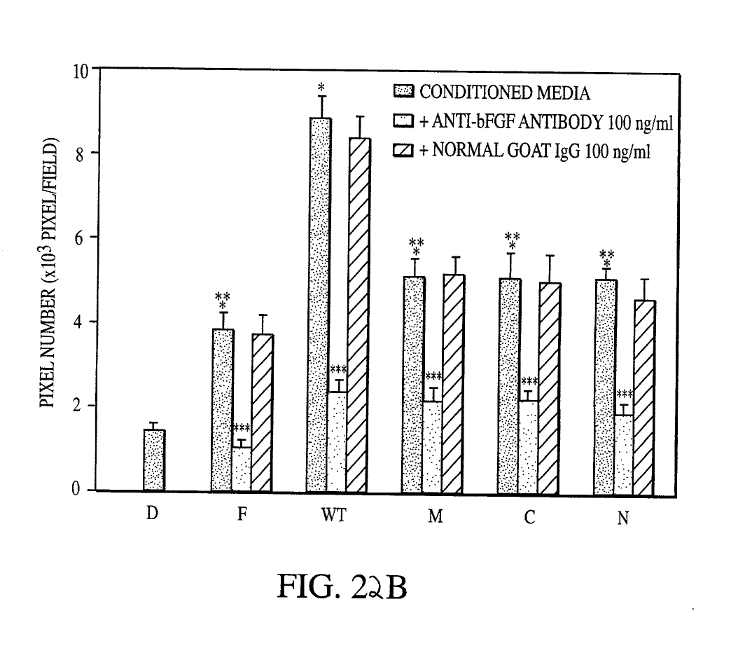

Tube forming assay. Matrigel is useful for studying HUVEC attachment and differentiation. Since Matrigel itself induces HUVEC differential activity, we used GFR Material to reduce the effect of growth factors from the Matrigel itself As shown in FIG. 22A, when HUVECs adhered on GFR Materiel, they aligned with one another and formed tubes resembling a capillary plexus under the influence of differential activity in the CM. Quantitative analysis of HUVEC tube formation (Denekamp J., Review article: angiogenesis, neovascular proliferation and vascular pathophysiology as targets for cancer therapy, Br J Radiol. 66:181-196 [1993]) revealed that WT-hPTTG-CM enhanced HUVEC tube formation compared to that observed when HUVECs were incubated in CM derived from other cell lines (FIG. 22B; p<0.01). The morphologic changes resembling capillary formation were suppressed by adding anti-bFGF antibody to each CM. Suppressive effects of anti-bFGF antibody of WT-hPTTG-CM, M-hPTTG-CM, C-CM and N-CM were, in the same order, 74%, 58%, 57%, and 62%.

The results of the assay of tube-formation of HUVEC cells further demonstrated that WT-hPTTG-CM induces strong angiogenic activity. As similar angiogenic activity was observed using M-hPTTG-CM to C-CM and N-CM, and these were all significantly lower than angiogenic activity mediated by Wt-hPTTG CM, it appears that the proline-rich domains of PTTG are important not only for transforming action, but also for PTTG- mediated angiogenic properties.

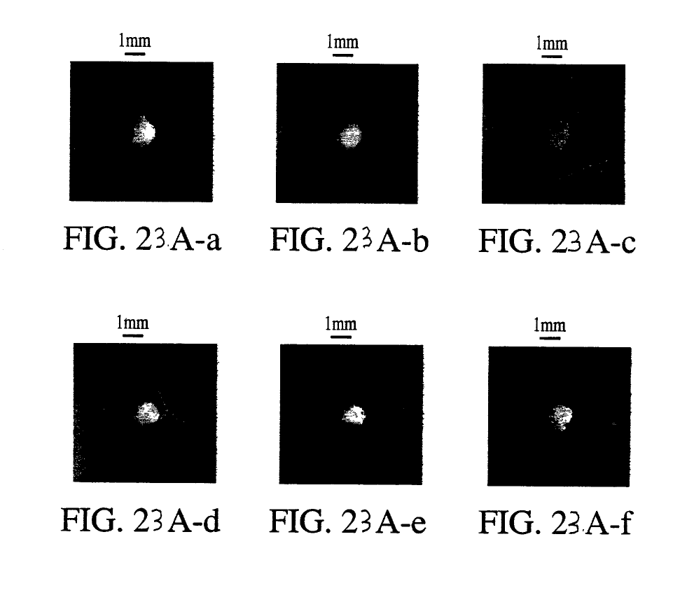

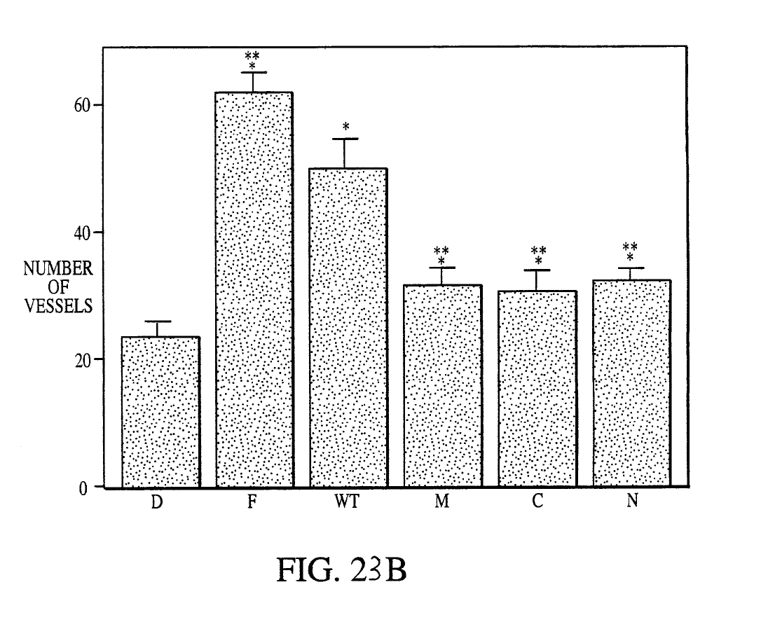

CAM assay. PTTG-mediated angiogenic activity was also examined in vivo by CAM assay. Chick CAM provides an ideal microenvironment to induce new vessel development from pre-existing vessels. We observed that CM from WT-hPTTG-transfected cells induced a spoke-wheel like appearance on the CAM and this effect was more marked than that observed with CM derived from other cell lines. Vessel growth of CAM was tested in vivo using 9-day-old chick egg embryos. Sample-soaked collagen sponges were loaded on CAM and neovascularization of surrounding collagen sponges evaluated after 4 days incubation. As shown in FIG. 23A, application of sponges presoaked in WT-hPTTG-CM induced a spoke-wheel like appearance which was more evident than CAM vessel formation after application of sponges immersed in the other CMs. The number of detectable blood vessels entering the collagen sponges were counted under stereomicroscopy, and as predicted, all CM samples derived from both transfected and non-transfected NIH-3T3 cells induced stronger angiogenic responses than did serum-free DMEM alone (p<0.01).

Application of sponges containing WT-hPTTG-CM to the CAM induced highest angiogenic activity (p<0.01), although higher angiogenic activity was observed when recombinant bFGF (1 μg/egg) was added to the CAM. In contrast to the in vitro assays, quantitative angiogenic activity of sponges soaked in WT-hPTTG derived CM was weaker than that observed after application of a sponge soaked in added bFGF (1 μg/egg). However, bFGF bioavailability in the recombinant peptide-soaked sponge and the CM-soaked sponge may differ accounting for this discrepancy.

We claim:

1. A method of modulating angiogenesis in a tissue comprising mammalian cells, comprising:
modulating PTTG gene expression and/or endogenous PTTG protein function in at least one of the cells, such that by
(A) inhibiting PTTG gene expression and/or endogenous PTTG protein function in at least one of the cells, bFGF production and/or secretion in the tissue is inhibited and angiogenesis in the tissue is thereby inhibited; or
(B) enhancing PTTG gene expression and/or endogenous PTTG protein function in at least one of the cells, bFGF production and/or secretion in the tissue is enhanced and angiogenesis in the tissue is thereby enhanced.

2. The method of claim 1, further comprising inhibiting angiogenesis by delivering a PTTG-specific antisense oligonucleotide to the cell(s).

3. The method of claim 2, wherein the antisense oligonucleotide specifically binds to a regulatory region in the PTTG promoter selected from the group consisting of SSCA, 8182, a cyclic-AMP responsive element, an estrogen responsive element, an insulin response element, SP1, and a GC Box.

4. The method of claim 1, further comprising inhibiting angiogenesis by interfering with SH3-mediated signal transduction by blocking specific binding to SH3-binding sites on endogenous PTTG protein molecules.

5. The method of claim 1, further comprising:
inhibiting angiogenesis by delivering to the cell(s) a composition comprising a PTTG carboxy-terminal-related polynucleotide, said polynucleotide being complexed with a cellular uptake-enhancing agent, in an amount and under conditions sufficient to allow the polynucleotide to enter the cell, whereby bFGF production and/or secretion is inhibited.

6. The method of claim 1, wherein the tissue comprises human cells.

7. The method of claim 1, wherein the tissue is a malignant tissue and angiogenesis is inhibited by (A).

8. The method of claim 1, wherein the tissue is retinal tissue and angiogenesis is inhibited by (A).

9. The method of claim 1, wherein the tissue is renal vascular tissue and angiogenesis is inhibited by (A).

10. The method of claim 5, further comprising administering the composition to a mammalian subject, such that the composition is delivered to the cell(s) in vivo.

11. The method of claim 5, wherein the polynucleotide is a DNA or DNA analog.

12. The method of claim 5, wherein the polynucleotide is an antisense oligonucleotide.

13. The method of claim 5, wherein the polynucleotide is a protein nucleic acid.

14. The method of claim 5, wherein the composition further comprises an expression vector comprising a promoter, and the PTTG carboxy-terminal-related polynucleotide is operatively linked to the promoter in a transcriptional unit.

15. The method of claim 14, wherein the polynucleotide encodes a PTTG carboxy-terminal peptide.

16. The method of claim 15, wherein the polynucleotide defines a nucleotide base sequence encoding a mammalian PTTG-C peptide selected from the group consisting of
(A) peptides having an amino acid sequence of (SEQ. ID. NO.:9), (SEQ. ID. NO.:16), or (SEQ. ID. NO.:17);
(B) mammalian PTTG-C peptides having at least about 60% sequence homology with any of (A); and
(C) peptide fragments of (A) or (B) that comprise at least 15 contiguous amino acid residues and that function to downregulate endogenous PTTG expression and/or PTTG function.

17. The method of claim 16, wherein the peptide fragment of (C) comprises a proline-rich region.

18. The method of claim 16, wherein the polynucleotide has a nucleotide sequence consisting of
(A) (SEQ. ID. NO.:10), (SEQ. ID. NO.:18), or (SEQ. ID. NO.:19)
(B) a degenerate coding sequence of any of (A);
(C) a sequence complementary to any of (A) or (B); or
(D) a polynucleotide fragment comprising at least 45 contiguous nucleotides of any of (A), (B) or (C).

19. The method of claim 1, further comprising:
inhibiting angiogenesis in the tissue by delivering to the cell(s) a composition comprising an expression vector comprising a promoter and a polynucleotide, said polynucleotide comprising a first DNA segment encoding a mammalian PTTG-C peptide, said polynucleotide being operatively linked to the promoter in a transcriptional unit, said PTTG-C peptide being selected from the group consisting of
(A) peptides having an amino acid sequence of (SEQ. ID. NO.:9), (SEQ. ID. NO.:16), or (SEQ. ID. NO.:17);
(B) mammalian PTTG-C peptides having at least about 60% sequence homology with any of (A); and
(C) peptide fragments of (A) or (B) that comprise at least 15 contiguous amino acid residues and that function to downregulate endogenous PTTG expression and/or PTTG function,
said expression vector being complexed with a cellular uptake-enhancing agent, in an amount and under conditions sufficient to enter the cell(s), such that the PTTG-C peptide is expressed in the cell(s), whereby bFGF production and/or secretion is inhibited.

20. The method of claim 19, wherein the peptide fragment of (C) comprises a proline-rich region.

21. The method of claim 19, wherein the polynucleotide further comprises a second DNA segment encoding an uptake-enhancing and/or importation-competent peptide segment.

22. The method of claim 19, further comprising administering the composition to a mammalian subject in need of treatment, such that the expression vector is delivered to the cell(s) in vivo.

23. The method of claim 1, further comprising:
inhibiting angiogenesis in the tissue by delivering to the cell(s) a composition comprising a PTTG carboxy terminal peptide, or a biologically functional fragment thereof, complexed with a cellular uptake-enhancing agent, in an amount and under conditions sufficient to enter the cell(s) whereby production and/or secretion of bFGF is inhibited.

24. The method of claim 23, wherein said uptake-enhancing agent is a polycationic lipid agent.

25. The method of claim 23, wherein said uptake enhancing agent comprises a cellular uptake-enhancing and/or importation-competent peptide segment.

26. The method of claim 1, further comprising:
inhibiting angiogenesis by delivering to the cell(s) a composition comprising a PTTG-C peptide being selected from the group consisting of
(A) peptides having an amino acid sequence of (SEQ. ID. NO.:9), (SEQ. ID. NO.:16), or (SEQ. ID. NO.:17);
(B) mammalian PTTG-C peptides having at least about 60% sequence homology with any of (A); and
(C) peptide fragments of (A) or (B) that comprise at least 15 contiguous amino acid residues and that function to downregulate endogenous PTTG expression and/or PTTG function,
said expression vector being complexed with a cellular uptake-enhancing agent, in an amount and under conditions sufficient to enter the cell(s), such that the PTTG-C peptide is expressed in the cell(s), whereby bFGF production and/or secretion is inhibited.

27. The method of claim 26, wherein the peptide fragment of (C) comprises a proline-rich region.

28. The method of claim 26, further comprising administering the composition to a human subject in need of treatment, such that the PTTG-C peptide is delivered to the cell(s) in vivo.

29. The method of claim 26, wherein said uptake enhancing agent comprises a polycationic lipid.

30. The method of claim 26, wherein said uptake enhancing agent comprises a cellular uptake-enhancing and/or importation-competent peptide segment.

31. The method of claim 1, further comprising enhancing angiogenesis in the tissue by delivering to the cell(s) a composition comprising an expression vector comprising a promoter and a polynucleotide, said polynucleotide comprising a first DNA segment encoding a mammalian PTTG peptide, said polynucleotide being operatively linked to the promoter in a transcriptional unit, said PTTG peptide being selected from the group consisting of
(A) peptides having an amino acid sequence of (SEQ. ID. NO.:2), (SEQ. ID. NO.:4), or (SEQ. ID. NO.:14); and
(B) mammalian PTTG peptides having at least about 60% sequence homology with any of (A),
said expression vector being complexed with a cellular uptake-enhancing agent, in an amount and under conditions sufficient to enter the cell(s), such that the PTTG peptide is overexpressed in the cell(s), whereby bFGF production and/or secretion is enhanced.

32. The method of claim 31, wherein the polynucleotide further comprises a second DNA segment encoding an uptake-enhancing and/or importation-competent peptide segment.

33. The method of claim 31, further comprising administering the composition to a mammalian subject in need of treatment, such that the expression vector is delivered to the cell(s) in vivo.

34. The method of claim 1, further comprising:
enhancing angiogenesis in the tissue by delivering to the cell(s) a composition comprising a PTTG peptide, or a biologically functional fragment thereof, complexed with a cellular uptake-enhancing agent, in an amount and under conditions sufficient to enter the cell(s) whereby production and/or secretion of bFGF is enhanced.

35. The method of claim 34, wherein said uptake-enhancing agent is a polycationic lipid agent.

36. The method of claim 35, wherein said uptake enhancing agent comprises a cellular uptake-enhancing and/or importation-competent peptide segment.

37. The method of claim 1, further comprising:
enhancing angiogenesis by delivering to the cell(s) a composition comprising a PTTG peptide being selected from the group consisting of
(A) peptides having an amino acid sequence of (SEQ. ID. NO.:2), (SEQ. ID. NO :4), or (SEQ. ID. NO.:14); and
(B) mammalian PTTG peptides having at least about 60% sequence homology with any of (A),
said expression vector being complexed with a cellular uptake-enhancing agent, in an amount and under conditions sufficient to enter the cell(s), such that the PTTG peptide is expressed in the cell(s), whereby bFGF production and/or secretion is enhanced.

38. The method of claim 37, further comprising administering the composition to a human subject in need of treatment, such that the PTTG peptide is delivered to the cell(s) in vivo.

39. The method of claim 37, wherein said uptake enhancing agent comprises a polycationic lipid.

40. The method of claim 37, wherein said uptake enhancing agent comprises a cellular uptake-enhancing and/or importation-competent peptide segment.

41. The method of claim 1, wherein the tissue is cardiovascular tissue and angiogenesis is enhanced by (B).

42. The method of claim 1, wherein the tissue is cerebrovascular tissue and angiogenesis is enhanced by (B).

43. A method of enhancing wound healing and/or tissue regeneration, comprising the method of claim 1, wherein angiogenesis in the wounded tissue is enhanced by (B).

44. The method claim 43, wherein the wounded tissue is hepatic tissue.

45. A method of limiting scar formation, comprising the method of claim 1, wherein the tissue is scar tissue and angiogenesis is inhibited in said tissue by (A).
